(19) United States
(12) Patent Application Publication
(10) Pub. No.: US 2007/0037855 A1
(43) Pub. Date: Feb. 15, 2007

(54) Compounds for inhibiting beta-amyloid production and methods of identifying the compounds

(76) Inventors: Michael J. Mullan, Tampa, FL (US); Daniel Paris, Sarasota, FL (US); Pancham Bakshi, Sarasota, FL (US)

Correspondence Address: KING & SPALDING LLP, 1180 PEACHTREE STREET, ATLANTA, GA, 30309-3521, US

(21) Appl. No.: 11/581,883

(22) Filed: Oct. 17, 2006

Related U.S. Application Data
(63) Continuation of application No. 11/328,470, filed on Jan. 9, 2006.
(60) Provisional application No. 60/642,268, filed on Jan. 7, 2005.
(60) Provisional application No. 60/669,055, filed on Apr. 7, 2005.

(51) Int. Cl. A61K 31/4439; A61K 31/4412; G01N 33/567

(52) U.S. Cl. 514/340; 514/350; 514/355; 435/7.2; 514/348

(57) ABSTRACT

Provided are compounds useful for treating diseases associated with a cerebral accumulation of Alzheimer's amyloid, such as Alzheimer's disease. Also provided are methods for screening for such compounds, by measuring capacitative calcium entry in cells which optionally overexpress APP or a fragment thereof. Also provided are methods of treating or reducing the risk of developing β-amyloid production, β-amyloid deposition, β-amyloid neurotoxicity (including abnormal hyperphosphorylation of tau) and microgliosis associated with cerebral accumulation of Alzheimer's amyloid by administering therapeutically effective amounts of compounds which decrease β-amyloid production and capacitative calcium entry in cells. Further provided are methods for diagnosing diseases associated with cerebral accumulation of Alzheimer's amyloid in animals or humans by administering diagnostically effective amounts of compounds which inhibit capacitative calcium entry in cells.

### CROSS REFERENCE TO RELATED APPLICATIONS

This application claims priority to U.S. Provisional Application No. 60/642,268 filed on Jan. 7, 2005 and U.S. Provisional Application No. 60/669,055 filed on Apr. 7, 2005.

### FIELD OF THE INVENTION

The present invention relates to compounds for the treatment of diseases associated with cerebral accumulation of Alzheimer's amyloid, such as Alzheimer's disease, screening methods for identifying the compounds, and methods of use of the compounds for the treatment and diagnosis of diseases associated with cerebral accumulation of Alzheimer's amyloid.

### DESCRIPTION OF RELATED ART

Alzheimer's disease (AD) is the most common neurodegenerative disorder of aging, afflicting approximately 1% of the population over the age of 65. Characteristic features of the disease include neurofibrillary tangles composed of abnormal tau protein, paired helical filaments, neuronal loss, and alteration in multiple neurotransmitter systems. The hyperphosphorylation of microtubule-associated tau protein is a known marker of the pathogenic neuronal pre-tangle stage in AD brain (Tan et al., “Microglial Activation Resulting from CD40R/CD40L Interaction after Beta-Amyloid Stimulation,” Science (1999) 286:2352-55).

A significant pathological feature of AD is an overabundance of diffuse and compact senile plaques in association with limbic areas of the brain. Although these plaques contain multiple proteins, their cores are composed primarily of β-amyloid protein, a 39-43 amino acid proteolytic fragment that is proteolytically derived from amyloid precursor protein (APP), a transmembrane glycoprotein. Additionally, C-terminal fragments (CTF) of APP are known to accumulate intraneuronally in AD.

β-amyloid is derived from APP, a single-transmembrane protein with a 590 to 680 amino acid extracellular amino terminal domain and an approximately 55 amino acid cytoplasmic tail. Messenger RNA from the APP gene on chromosome 21 undergoes alternative splicing to yield eight possible isoforms, three of which (the 695, 751 and 770 amino acid isoforms) predominate in the brain. APP undergoes proteolytic processing via three enzymatic activities, termed α-, β- and γ-secretase. Alpha-secretase cleaves APP at amino acid 17 of the β-amyloid domain, thus releasing the large soluble amino-terminal fragment α-APP for secretion. Because a-secretase cleaves within the β-amyloid domain, this cleavage precludes β-amyloid formation. Alternatively, APP can be cleaved by β-secretase to define the amino terminus of β-amyloid and to generate the soluble amino-terminal fragment β-APP. Subsequent cleavage of the intracellular carboxy-terminal domain of APP by γ-secretase results in the generation of multiple peptides, the two most common being a 40 amino acid β-amyloid (Aβ1-40) and 42 amino acid β-amyloid (Aβ1-42). Aβ1-40 comprises 90-95% of the secreted β-amyloid and is the predominant species recovered from cerebrospinal fluid (Seubert et al., Nature, 359:325-7, 1992). In contrast, less than 10% of secreted β-amyloid is Aβ1-42. Despite the relative paucity of Aβ1-42 production, Aβ1-42 is the predominant species found in plaques and is deposited initially, perhaps due to its ability to form insoluble amyloid aggregates more rapidly than Aβ1-40 (Jarrett et al., Biochemistry, 32:4693-7, 1993). The abnormal accumulation of β-amyloid in the brain is believed to be due to decreased clearance of β-amyloid from the brain to the periphery or excessive production of β-amyloid. Various studies suggests excessive production of β-amyloid is due to either overexpression of APP or altered processing of APP, or mutation in the γ secretases or APP responsible for β-amyloid formation.

β-Amyloid peptides are thus believed to play a critical role in the pathobiology of AD, as all the mutations associated with the familial form of AD result in altered processing of these peptides from APP. Indeed, deposits of insoluble, or aggregated, fibrils of β-amyloid in the brain are a prominent neuropathological feature of all forms of AD, regardless of the genetic predisposition of the subject. It also has been suggested that AD pathogenesis is due to the neurotoxic properties of β-amyloid. The cytotoxicity of β-amyloid was first established in primary cell cultures from rodent brains and also in human cell cultures. The work of Mattson et al. (J. Neurosci., 12:376-389, 1992) indicates that β-amyloid, in the presence of the excitatory neurotransmitter glutamate, causes an immediate pathological increase in intracellular calcium, which is believed to be very toxic to the cell through its greatly increased second messenger activities.

Concomitant with β-amyloid production and β-amyloid deposition, there exists robust activation of inflammatory pathways in AD brain, including production of pro-inflammatory cytokines and acute-phase reactants in and around β-amyloid deposits (McGeer et al., J. Leukocyte Biol., 65:409-15, 1999). Activation of the brain's resident innate immune cells, the microglia, is thought to be intimately involved in this inflammatory cascade. It has been demonstrated that reactive microglia produce pro-inflammatory cytokines, such as inflammatory proteins and acute phase reactants, such as alpha-1-antichymotrypsin, transforming growth factor β, apolipoprotein E and complement factors, all of which have been shown to be localized to β-amyloid plaques and to promote β-amyloid plaque “condensation” or maturation (Nilsson et al., J. Neurosci. 21:1444-5, 2001), and which at high levels promote neurodegeneration. Epidemiological studies have shown that patients using non-steroidal anti-inflammatory drugs (NSAIDS) have as much as a 50% reduced risk for AD (Rogers et al., Neurobiol. Aging 17:681-6, 1996), and post-mortem evaluation of AD patients who have undergone NSAID treatment has demonstrated that risk reduction is associated with diminished numbers of activated microglia (Mackenzie et al., Neurology 50:986-90, 1998). Further, when Tg APPsw mice, a mouse model for Alzheimer's disease, are given an NSAID (ibuprofen), these animals show reduction in β-amyloid deposits, astrocytosis, and dystrophic neurites correlating with decreased microglial activation (Lim et al., J. Neurosci. 20:5709-14, 2000).

At present, treatment for AD is limited. However, there are several drugs approved by the FDA to improve or stabilize symptoms of AD (Alzheimer's Disease Medications Fact Sheet: (July 2004) U.S. Department of Health and Human Services), including Aricept® (donepezil), Exelon® (rivastigmine), Reminyl® (galantamine) Cognex® (tacrine) and Namenda® (memantine). The effects with many drugs currently in use is small (Tariot et al., JAMA (2004), 291: 317-24). Treatments for AD remain a largely unmet clinical need.

U.S. Patent Application No. 2005009885 (Jan. 13, 2005) (Mullan et al.) discloses a method for reducing beta-amyloid deposition using nilvadipine, as wells as methods of diagnosing cerebral amyloidogenic diseases using nilvadipine. Nimodipine has been studied for the treatment of dementia. Fritze et al., J. Neural Transm. (1995) 46: 439-453; and Forette et al. Lancet (1998) 352: 1347-1351).

Augmentation of capacitative calcium entry (CCE) through the identification of agonist of plasma membrane store-operated calcium channels that mediate CCE, has been suggested as a treatment for AD (Tanzi et al. Neuron (2000) 27: 561-572). U.S. Patent Application Publication No. 20020015941 (Feb. 7, 2002) discloses a method for the treatment of a neurodegenerative disease such as AD involving administering an agent which is capable of potentiating CCE.

There continues to be a need to identify compounds that can treat the inexorable progression of brain degeneration which is a hallmark of AD, wherein the treatment addresses β-amyloid production and the concomitant β-amyloid deposition, β-amyloid neurotoxicity (including abnormal hyperphosphorylation of tau), microglial-activated inflammation, and altered or over expression of APP which is seen in AD patients.

### SUMMARY

It has been surprisingly discovered that compounds which decrease capacitative calcium entry in mammalian cells that overexpress amyloid precursor protein (APP) can decrease β-amyloid production in the cells. It also have been discovered that such compounds can be used in methods for the treatment of diseases associated with the accumulation of β-amyloid.

Entry of Ca²⁺ from the extracellular space occurs through three classes of Ca²⁺ permeable gates: voltage-dependent Ca²⁺ channels, ligand-gated Ca²⁺-permeable cation channels, and the so-called capacitative calcium entry channels. Bimbaumer, et al., Proc. Natl. Acad. Sci. USA 24; 93(26): 15195-15202 (1996). Capacitative calcium entry (CCE) is one of the most prevalent mechanisms of cellular Ca²⁺ signaling and, unlike the other calcium channels, CCE is ubiquitous in cells. Capacitative calcium entry involves the activation of plasma membrane calcium channels to cause the influx of extracellular calcium, in response to a fall in Ca²⁺ concentration within the lumen of Ca²⁺ storing organelles, most commonly components of the endoplasmic reticulum. The endoplasmic reticulum is believed to signal the plasma membrane calcium channels in the process of capacitative calcium entry. Capacitative calcium entry replenishes cellular Ca²⁺ stores at a rapid rate, for example, as required following transient receptor activation by neurotransmitters. J. W. Putney, Jr., Molecular Inventions, 1:84, June, 2001. Cells which overexpress APP or fragment thereof surprisingly can respond to CCE inhibitors by reducing β-amyloid production. Such CCE inhibitors are useful in reducing β-amyloid production and treating diseases associated with β-amyloid accumulation.

Provided are compounds which decrease capacitative calcium entry, for example, by about 5%, 10%, 15%, 20%, 22%, 25%, 28%, 30%, 40%, 50%, 60% or more in cultured mammalian cells, for example cells which overexpress amyloid precursor protein (APP), wherein optionally the compounds also decrease β-amyloid production. Such compounds can be used in the methods disclosed herein.

Also provided is an in vitro method of screening for a compound for use in treating animals or humans afflicted with a disease associated with cerebral accumulation of Alzheimer's amyloid, such as Alzheimer's disease (AD), comprising exposing cells to a test compound; measuring capacitative calcium entry (CCE) in the cells, wherein the cells optionally overexpress APP or a fragment thereof; and detecting a decrease in CCE of at least about 5%, 10%, 15%, 20% or more in the cells, as measured, e.g., in comparison to unexposed cells, as an indicator of the therapeutic usefulness of the compound to treat animals or humans afflicted with a disease associated with cerebral accumulation of Alzheimer's amyloid. The compounds which are tested for their ability to inhibit CCE are screened, for example, in concentrations of about 1 nM to 10 mM, about 500 nM to 50 μM, or about 5 μM to 30 μM. The cultured cells are, for example, exposed to the test compound for at least about 15 minutes, 30 minutes, 60 minutes or more. The cells that can be used in the CCE assay may be selected from mammalian or non-mammalian cells, including Chinese hamster ovary cells that overexpress APP75 1, human neuronal precursor cells (HNPC); primary culture of human astrocytes; neuroblastoma cells; human brain microvascular endothelial primary culture; or human umbilical cord endothelial cells (HUVEC).

Optionally or additionally, in an in vitro assay method to identify compounds useful in the treatment of diseases associated with the accumulation of β-amyloid, an assay to determine the compounds' ability to decrease β-amyloid production is conducted. For example, the test compound is exposed to cells that overexpress APP or a fragment thereof, β-amyloid production in the cells is measured; and a decrease in β-amyloid production of e.g., at least about 20% more in the cells that overexpress APP or a fragment thereof is detected as an indicator of the therapeutic usefulness of the compound to treat animals or humans afflicted with a disease associated with cerebral accumulation of Alzheimer's amyloid. The assay is conducted using cells that overexpress APP or a fragment thereof available in the art such as Chinese hamster ovary cells that overexpress APP751. The β-amyloid measured, is, e.g., Aβ1-40, Aβ1-42, or total Aβ1-40+Aβ1-42. A decrease in the production of Aβ1-40 and/or Aβ1-42, and in particular, total Aβ1-40+Aβ1-42, of, e.g., at least about 5%, 10%, 15%, 20%, 25%, 30%, 50%, or more, indicates the therapeutic effectiveness of the compound to treat animals or humans afflicted with a disease associated with cerebral accumulation of Alzheimer's amyloid. The β-amyloid concentrations can be measured for example, intracellularly or, e.g., extracellularly in the culture medium.

The compounds which are tested for their ability to inhibit CCE as well as to reduce AO production are screened in a range of concentrations, for example, about 1 nM to 10 mM, about 500 nM to 50 μM, or about 5 μM to 30 μM.

Also provided is a method of treating a disease associated with cerebral accumulation of β-amyloid in animals or humans afflicted with the disease, such as AD, by administering a therapeutically effective amount of at least one compound that decreases CCE by at least about 5%, 10%, 15%, 20% or more in cells, that for example overexpress APP or a fragment thereof, and/or optionally reduces B-amyloid production by at least about 5%, 10%, 15%, 20%, 25%, 30%, 50%, or more in cells that overexpress APP or a fragment thereof, as can be measured, for example in a culture medium comprising the cells. The method may in one embodiment include one or more of reducing β-amyloid production, β-amyloid deposition, β-amyloid neurotoxicity (including abnormal hyperphosphorylation of tau) and microgliosis. Because most diseases having cerebral accumulation of Alzheimer's amyloid, such as AD, are chronic, progressive, intractable brain dementias, it is contemplated that the duration of treatment with at least one of the active agents can optionally last for up to the lifetime of the animal or human.

Further provided is a method for diagnosing diseases associated with cerebral accumulation of Alzheimer's amyloid, such as AD, in an animal or human, or determining if the animal or human is at risk for developing cerebral accumulation of Alzheimer's amyloid, the method comprising: taking a first measurement of β-amyloid concentration in a body fluid such as plasma, serum, whole blood, urine or cerebral spinal fluid (CSF) of the animal or human; administering to the animal or human a diagnostically effective amount in unit dosage form of a compound that decreases CCE by at least about 5%, 10%, 15%, 20% or more in cultured cells that for example overexpress APP or a fragment thereof, and/or optionally reduces β-amyloid production, for example, by at least about 5%, 10%, 15%, 20%, 25%, 30%, 50%, as measured for example in a culture medium comprising the cells; taking a second measurement of β-amyloid concentration from plasma, serum, whole blood, urine or CSF of the animal or human at a later time; and calculating the difference between the first measurement and the second measurement. A change in the concentration of β-amyloid or fragment thereof in plasma, serum, whole blood, urine or CSF in the second measurement compared to the first measurement, in particular an increase in concentration, indicates a risk of developing or a possible diagnosis of a disease associated with cerebral accumulation of Alzheimer's amyloid in the animal or human.

Also provided is a method for treating head injury, and optionally reducing the risk of β-amyloid production, β-amyloid deposition, β-amyloid neurotoxicity (including abnormal hyperphosphorylation of tau) or microgliosis, in animals or humans suffering from traumatic brain injury, the method comprising administering to the animal or human immediately after the head injury a therapeutically effective amount in unit dosage form of a compound that decreases CCE by at least about 5%, 10%, 15%, 20% or more in cultured cells for example those cells which overexpress APP or a fragment thereof, and/or optionally reduce β-amyloid production by at least about 5%, 10%, 15%, 20%, 25%, 30%, 50%, as measured, for example in a culture medium comprising the cells, and then optionally continuing treatment with the compound for a prescribed period of time thereafter.

Cells which overexpress APP or a fragment thereof which can be used according to the methods disclosed herein include mammalian or non-mammalian cells including but not limited to 7W WT APP751 Chinese hamster ovary cells. APP which is overexpressed can include, without limitation, APP751. Cells which can be used to measure changes in CCE include non-mammalian and mammalian cells, such as epithelial or endothelial cells.

A variety of compounds are provided, as well as methods for their use in the treatment and diagnosis of diseases associated with cerebral accumulation of Alzheimer's amyloid. In one embodiment, the compound is a dihydropyridine which is optionally other than nilvadipine, nimodipine or nitrendipine. In another embodiment, the compound is an iunidazole compound. In a further embodiment, the compound is an isoquinoline alkaloid compound. In another embodiment, the compound is a calmodulin-mediated enzyme activation inhibitor. In yet another embodiment, the compound is an inhibitor of kinase activity of the platelet-derived growth factor (PDGF) receptor. In yet another embodiment, the compound is an NF-kB activation inhibitor. In another embodiment, the compound is a diterpene or triterpene compound. In yet another embodiment, the compound is a quinazoline compound. In one embodiment, the compound is a sesquiterpene lactone. In another embodiment, the compound is an inhibitor of IKK-2. In one preferred embodiment, said compound decreases CCE, for example, by at least about 10% or more in the medium of cultured cells that for example overexpress APP or a fragment thereof, and/or optionally reduces β amyloid production, for example, by at least about 20% or more, in cells that overexpress APP or a fragment thereof.

In the embodiment, compounds which can be used for the treatment and diagnosis of diseases associated with cerebral accumulation of Alzheimer's amyloid in the embodiments disclosed herein are provided that include, without limitation:

SKF96365 (1-[beta-[3-(4-methoxyphenyl)propoxy]4-methoxyphenethyl]-1H-imidazole hydrochloride), econazole, clotrimazole;

SR 33805 (3,4-dimethoxy-N-methyl-N-[3-[4-[[1-methyl-3-(1-methylethyl)-1H-in-dol-2-yl]sulfonyl]phenoxy]propyl]benzeneethanamine oxalate);

loperamide;

tetrandrine;

R24571 (1-[bis(p-chlorophenyl)methyl]-3-[2-(2,4-di-chloro-β-(2,4-dichlorobenzyl-oxy)phenethyl)]-imidazolium chloride);

amlodipine;

nitrendipine;

MRS 1845 (N-propargylnitrendipine);

tyrphostin A9;

BTB 14328 (diethyl 4-(4-chlorophenyl)-2,6-dimethyl-1,4-dihydropyridine-3,5-dicarboxylate);

CD 04170 (diethyl 4-{5-[3,5-di(trifluoromethyl)phenyl]-2-furyl}-2,6-dimethyl-1,4-dihydro-pyridine-3,5-dicarboxylate);

HTS 01512 (1-cyclohexyl-5-phenyl-1,6-dihydro-2,3-pyridinedione);

HTS 07578 (4-(1,3-diphenyl-1H-pyrazol-4-yl)-2-oxo-6-phenyl-1,2-dihydro-3-pyridinecarbonitrile);

HTS 10306 (2-oxo-6-phenyl-4-(2-thienyl)-1,2-dihydro-3-pyridinecarbonitrile);

JFD 01209 (diethyl 4-(4-bromophenyl)-2,6-dimethyl-1,4-dihydropyridine-3,5-dicarboxylate);

JFD 03266 (diethyl 2,6-dimethyl-4-(4-nitrophenyl)-1,4-dihydropyridine-3,5-dicarboxylate;

JFD 03274 (diethyl 4-(3-chlorophenyl)-2,6-dimethyl-1,4-dihydropyridine-3,5-dicarboxylate);

JFD 03282 (diethyl 2,6-dimethyl-4-(4-methylphenyl)-1,4-dihydropyridine-3,5-dicarboxylate);

JFD 03292 (4-(3,4-dichlorophenyl)-2,6-dimethyl-1,4-dihydropyridine-3,5-dicarbonitrile;

JFD 03293 (dimethyl 4-(3,4-dichlorophenyl)-2,6-dimethyl-1,4-dihydropyridine-3,5-dicarboxylate);

JFD 03294 (diethyl 4-(3,4-dichlorophenyl)-2,6-dimethyl-1,4-dihydropyridine-3,5-dicarboxylate);

JFD 03305 (diethyl 4-(2-chlorophenyl)-2,6-dimethyl-1,4-dihydropyridine-3,5-dicarboxylate);

JFD 03311 (diethyl 2,6-dimethyl-4-(2-nitrophenyl)-1,4-dihydropyridine-3,5-dicarboxylate);

JFD 03318 (diethyl 4-(4-fluorophenyl)-2,6-dimethyl-1,4-dihydropyridine-3,5-dicarboxylate);

PD 00463 (1-[4-(4-chlorophenoxy)phenyl]-4-phenyldihydropyridine-2,6(1H,3H)-dione);

RJC 03403 (diethyl 4-(2,4-dichlorophenyl)-2,6-dimethyl-1,4-dihydro-3,5-pyridinedicarboxylate);

RJC 03405 (diethyl 2,6-dimethyl-4-{5-[2-(trifluoromethyl)phenyl]-2-furyl}-1,4-dihydro-3,5-pyridinedicarboxylate);

RJC 03413 (diethyl 4-(2-chloro-4-methoxyphenyl)-2,6-dimethyl-1,4-dihydro-3,5-pyridinedicarboxylate);

RJC 03423 (dimethyl 4-(2,4-dichlorophenyl)-2,6-dimethyl-1,4-dihydro-3,5-pyridinedicarboxylate);

SEW 02070 (dimethyl 4-{5-[2-(methoxycarbonyl)-3-thienyl]-2-furyl}-2,6-dimethyl-1,4-dihydropyridine-3,5-dicarboxylate);

XBX 00343 (diethyl 2,6-dimethyl-4-(3-nitrophenyl)-1,4-dihydropyridine-3,5-dicarboxylate);

R-niguldipine,

(S)-(+)-niguldipine,

artemisinin;

celastrol;

6-amino-4-(4-phenoxyphenylethylamino)quinazoline;

isohelenin;

kamebakaurin;

parthenolide; and

IKK-2 Inhibitor IV;

or salts, esters, prodrugs, stereoisomers, or derivatives thereof.

Preferred are those compounds that decrease CCE, for example, by 10% or more in cultured cells which for example overexpress APP or a fragment thereof, and optionally reduce β-amyloid production, e.g., production of total Aβ₁₋₄₀ and Aβ₁₋₄₂, by at least about 20% or more in cells that overexpress APP or a fragment thereof

In one embodiment, the compound is one of the following compounds:

HTS 01512 (1-cyclohexyl-5-phenyl-1,6-dihydro-2,3-pyridinedione):

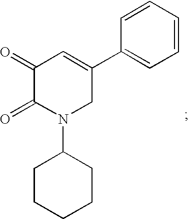

BTB 14328 (diethyl 4-(4-chlorophenyl)-2,6-dimethyl-1,4-dihydropyridine-3,5-dicarboxylate):

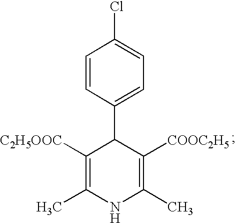

CD 04170 (diethyl 4-{5-[3,5-di(trifluoromethyl)phenyl]-2-furyl}-2,6-dimethyl-1,4-dihydro-pyridine-3,5-dicarboxylate):

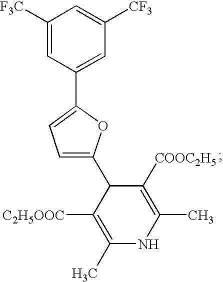

JFD 03292 (4-(3,4-dichlorophenyl)-2,6-dimethyl-1,4-dihydropyridine-3,5-dicarbonitrile:

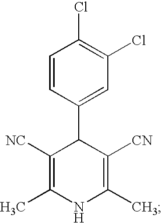

PD 00463 (1-[4-(4-chlorophenoxy)phenyl]-4-phenyldihydropyridine-2,6(1H,3H)-dione):

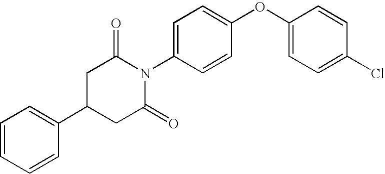

In another embodiment, the compound is one of the following compounds:

Diethyl 4-(2-bromophenyl)-1,4-dihydro-2,6-dimethylpyridine-3,5-dicarboxylate:

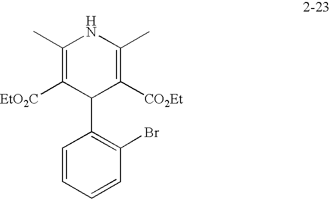

Diethyl 4-(2-fluorophenyl)-1,4-dihydro-2,6-dimethylpyridine-3,5-dicarboxylate:

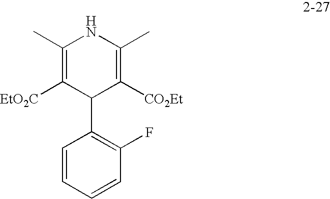

Di-tert-butyl 4-(2-fluorophenyl)-1,4-dihydro-2,6-dimethylpyridine-3,5-dicarboxylate:

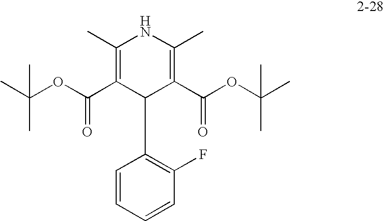

Diethyl 1,4-dihydro-2,6-dimethyl-4-(2-nitrophenyl)pyridine-3,5-dicarboxylate:

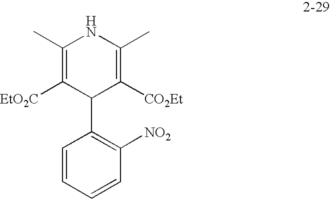

Di-tert-butyl 4-(2-bromophenyl)-1,4-dihydro-2,6-dimethylpyridine-3,5-dicarboxylate:

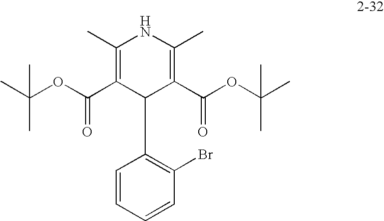

Di-tert-butyl 1,4-dihydro-2,6-dimethyl-4-(2-nitrophenyl)pyridine-3,5-dicarboxylate:

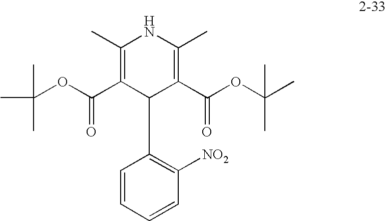

Di-tert-butyl 4-(4-bromo-2-fluorophenyl)-1,4-dihydro-2,6-dimethylpyridine-3,5-dicarboxylate:

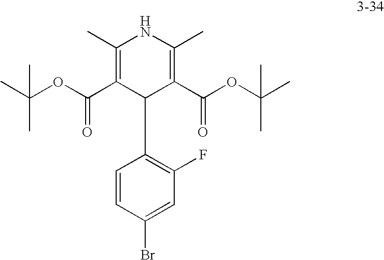

Bis(2-methoxyethyl) 4-(4-bromophenyl)-1,4-dihydro-2,6-dimethylpyridine-3,5-dicarboxylate:

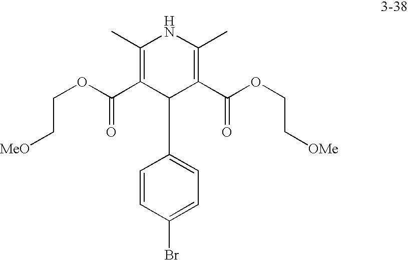

Diethyl 4-(5-bromo-2-fluorophenyl)-1,4-dihydro-2,6-dimethylpyridine-3,5-dicarboxylate

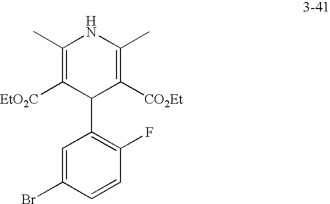

In another embodiment, a method is provided for treating a disease associated with cerebral accumulation of Alzheimer's amyloid, comprising administering to the animal or human a therapeutically effective amount of at least one active agent such as SKF96365, econazole, clotrimazole, SR 33805, loperamide, tetrandrine, R24571, amlodipine, nitrendipine, MRS 1845, tyrphostin A9, BTB 14328, CD 04170, HTS 01512, HTS 07578, HTS 10306, JFD 01209, JFD 03266, JFD 03274, JFD 03282, JFD 03292, JFD 03293, JFD 03294, JFD 03305, JFD 03311, JFD 03318, PD 00463, RJC 03403, RJC 03405, RJC 03413, RJC 03423, SEW 02070, XBX 00343, R-niguldipine, (S)-(+)-niguldipine, artemisinin, celastrol, 6-amino4-(4-phenoxyphenylethylamino)quinazoline, isohelenin, kamebakaurin, parthenolide, IKK-2 Inhibitor IV, 2-23, 2-27, 2-28, 2-29, 2-32, 2-33, 3-34, 3-38, 3-41, a compound as disclosed in Tables 1, 2 or 3 herein, or a compound of Formula I, II, III, IV, V, VI, VII, VIII, IX, X, or XI or other compound disclosed herein, or a salt, prodrug or derivative thereof. Preferably the active agent opposes the pathophysiological effects of the cerebral accumulation of Alzheimer's amyloid, and may, for example, reduce β-amyloid production, β-amyloid deposition, β-amyloid neurotoxicity and/or microgliosis in animals and humans afflicted with the disease.

In another embodiment, a diagnostic method for a disease associated with cerebral accumulation of Alzheimer's amyloid in an animal or human is provided, comprising: taking a first measurement of plasma, urine, serum, whole blood, or cerebral spinal fluid (CSF) concentration of β-amyloid in the peripheral circulation of the animal or human; administering a diagnostically effective amount in unit dosage form of at least one active agent selected from the group consisting of SKF96365, econazole, clotrimazole, SR33805, loperamide, tetrandrine, R24571, amlodipine, nitrendipine, MRS1845, tyiphostin A9, BTB 14328, CD 04170, HTS 01512, HTS 07578, HTS 10306, JFD 01209, JFD 03266, JFD 03274, JFD 03282, JFD 03292, JFD 03293, JFD 03294, JFD 03305, JFD 03311, JFD 03318, PD 00463, RJC 03403, RJC 03405, RJC 03413, RJC 03423, SEW 02070, XBX 00343, R-niguldipine, (S)-(+)-niguldipine, artemisinin, celastrol, 6-amino-4-(4-phenoxyphenylethylamino)quinazoline, isohelenin, kamebakaurin, parthenolide, IKK-2 Inhibitor IV, 2-23, 2-27, 2-28, 2-29, 2-32, 2-33, 3-34, 3-38, 3-41, a compound as disclosed in Tables 1, 2 or 3 herein, or a compound of Formula I, II, III, IV, V, VI, VII, VIII, IX, X, or XI or other compound disclosed herein, or salt, prodrug or derivative thereof, to the animal or human; taking a second measurement of plasma, serum, whole blood, urine or CSF concentration of β-amyloid in the peripheral circulation of the animal or human; and calculating the difference between the first measurement and the second measurement, wherein a change in the plasma, serum, whole blood, urine or CSF concentration of β-amyloid in the second measurement compared to the first measurement, in particular and increase in concentration, indicates a possible diagnosis of a disease associated with cerebral accumulation of Alzheimer's amyloid in the animal or human.

In a further embodiment, a method is provided for treating traumatic brain injury, comprising administering to the animal or human a therapeutically effective amount in unit dosage form of at least one active agent selected from the group consisting of SKF96365, econazole, clotrimazole, SR33805, loperamide, tetrandrine, R24571, amlodipine, nitrendipine, MRS1845, tyrphostin A9, BTB 14328, CD 04170, HTS 01512, HTS 07578, HTS 10306, JFD 01209, JFD 03266, JFD 03274, JFD 03282, JFD 03292, JFD 03293, JFD 03294, JFD 03305, JFD 03311, JFD 03318, PD 00463, RJC 03403, RJC 03405, RJC 03413, RJC 03423, SEW 02070, XBX 00343, R-niguldipine, (S)-(+)-niguldipine, artemisinin, celastrol, 6-amino-4-(4-phenoxyphenylethylamino)quinazoline, isohelenin, kamebakaurin, parthenolide, IKK-2 Inhibitor IV, 2-23, 2-27, 2-28, 2-29, 2-32, 2-33, 3-34, 3-38, 3-41, a compound as disclosed in Tables 1, 2 or 3 herein, or a compound of Formula I, II, III, IV, V, VI, VII, VIII, IX, X, or XI or other compound disclosed herein, or salt, prodrug or derivative thereof. In one embodiment, the administration of the active agent begins immediately following the injury. In one embodiment, the compound reduces the risk of β-amyloid production, Aβ deposition, β-amyloid neurotoxicity and/or microgliosis.

The therapeutically effective amount of compound that is administered e.g. in unit dosage form to animals or humans afflicted with a cerebral amyloidogenic disease or suffering from a traumatic brain injury, as well as administered for the purpose of determining the risk of developing and/or a diagnosis of a cerebral amyloidogenic disease in an animal or human, according to the methods of the present invention, can range from for example from about 0.05 mg to 20 mg per day, about 2 mg to 15 mg per day about 4 mg to 12 mg per day, or about 8 mg per day. The daily dosage in one embodiment can be administered in a single unit dose or divided into two, three or four unit doses per day.

In one embodiment, a method for treating a disease associated with cerebral accumulation of Alzheimer amyloid is provided, comprising administering to an animal or human a therapeutically effective amount of a compound that decreases capacitative calcium entry by at least about 10% or more in cells which optionally overexpress APP or a fragment thereof. Optionally, the cells are Chinese hamster ovary cells that overexpress APP751, or are selected from human neuronal precursor cells (HNPC); primary culture of human astrocytes; neuroblastoma cells; human brain microvascular endothelial primary culture; or human umbilical cord endothelial cells (HUVEC). In one embodiment, the compound is administered in an amount of about 0.02 to 1000 mg per unit dose; or about 0.5 to 500 mg per unit dose. In one embodiment, the compound is other than nilvadipine or a free base or a pharmaceutically acceptable salt thereof. In one embodiment, the compound is other than as described in U.S. Pat. Publ. No. 2005/0009885, published Jan. 13, 2005. In another embodiment, the compound is other than nilvadipine, nimodipine or nitrendipine. In another embodiment, the compound is other than nilvadipine, nimodipine or nitrendipine or a pharmaceutically acceptable salt, or free base thereof. In another embodiment, the compound is other than nilvadipine, nimodipine or nitrendipine or prodrug thereof.

In another embodiment, there is provided a method for diagnosing a disease associated with cerebral accumulation of Alzheimer amyloid in an animal or human, comprising: taking a first measurement of plasma, urine, serum, whole blood, or cerebral spinal fluid (CSF) concentration of β-amyloid in the peripheral circulation of the animal or human; administering to the animal or human a diagnostically effective amount of a compound that decreases capacitative calcium entry by at least about 10% or more in cells; taking a second measurement of plasma, serum, whole blood, urine or CSF concentration of β-amyloid in the peripheral circulation of the animal or human; and calculating the difference between the first measurement and the second measurement, wherein a change in the plasma, serum, whole blood, urine or CSF concentration of β-amyloid in the second measurement compared to the first measurement indicates a possible diagnosis of a disease associated with cerebral accumulation of Alzheimer amyloid in the animal or human. The cells may be selected from Chinese hamster ovary cells that overexpress APP751, or selected from human neuronal precursor cells (HNPC); primary culture of human astrocytes; neuroblastoma cells; human brain microvascular endothelial primary culture; or human umbilical cord endothelial cells (HUVEC). In one embodiment, the compound is other than nilvadipine or a free base or a pharmaceutically acceptable salt thereof. In one embodiment, the compound is other than as described in U.S. Pat. Publ. No. 2005/0009885, published Jan. 13, 2005.

In another embodiment, a method of treatment of an animal or human suffering from traumatic brain injury is provided, comprising administering a therapeutically effective amount of a compound that decreases capacitative calcium entry by at least about 10% or more in cells, such as Chinese hamster ovary cells that overexpress APP751; human neuronal precursor cells (HNPC); primary culture of human astrocytes; neuroblastoma cells; human brain microvascular endothelial primary culture; or human umbilical cord endothelial cells (HUVEC). In one embodiment, the compound is other than nilvadipine or a free base or a pharmaceutically acceptable salt thereof. In one embodiment, the compound is other than as described in U.S. Pat. Publ. No. 2005/0009885, published Jan. 13, 2005. The duration of treatment with the compound lasts for example, about one hour to one week; about one week to six months; or about six months to two years.

The disease associated with cerebral accumulation of Alzheimer's amyloid is for example, Alzheimer's disease, cerebral amyloid angiopathy, hereditary cerebral hemorrhage with amyloidosis Dutch-type, other forms of familial Alzheimer's disease and familial cerebral Alzheimer's amyloid angiopathy. Cerebral amyloidogenic diseases that can be treated or diagnosed include transmissible spongiform encephalopathy, scrapie, traumatic brain injury, cerebral amyloid angiopathy, and Gerstmann-Straussler-Scheinker syndrome.

### BRIEF DESCRIPTION OF THE DRAWINGS

FIGS. 1A-D are bar graphs showing the effect of various calcium channel blockers, such as SKF 96365, nilvadipine, nitrendipine and amlodipine, on Aβ1-40 production by 7W WT APP 751 Chinese hamster ovary (7W WT APP 751 CHO) cells. FIG. 1A shows the effect of calcium channel blocker treatment after 4 hours. FIG. 1B shows the effect of calcium channel blocker treatment after 24 hours. FIG. 1C shows the effect of calcium channel blocker treatment plated at low density after 24 hours. FIG. 1D shows the effect of calcium channel blocker treatment plated at low density after 48 hours.

FIG. 2 is a bar graph showing the effect of three CCE inhibitors, SKF96365, econazole and tyrphostin A9, on Aβ1-40, Aβ1-42 and total β-amyloid production by 7W WT APP751 CHO cells.

FIG. 3 is a bar graph showing the effect of various dihydropyridine calcium channel blockers, such as nilvadipine, nitrendipine and MRS 1835, on Aβ1-40, Aβ1-42 and total β-amyloid production by 7W WT APP751 CHO cells.

FIG. 4 is a bar graph showing the effect of various non-dihydropyridine and dihydropyridine calcium channel blockers, such as SR 33805, MRS 1845, loperamide, clotrimazole and tetrandine, on Aβ1-40, Aβ1-42 and total β-amyloid production by 7W WT APP751 CHO cells.

FIGS. 5A-B are bar graphs showing the effect of treating 7W WT APP751 CHO cells for 24 hours with various dihydropyridine compounds (obtained from Maybridge; England) on Aβ1-40, Aβ1-42 and total β-amyloid production.

FIG. 6 is a bar graph showing the effect of various NF-kB activation inhibitors on Aβ1-40, Aβ1-42 and total β-amyloid production by 7W WT APP751 CHO cells.

FIG. 7A is a graph showing that compounds which inhibit CCE in CHO cells also inhibit total Aβ production.

FIG. 7B is a list of compounds represented in FIG. 7A.

FIG. 8A is a graph showing that compounds which inhibit CCE in CHO cells also inhibit Aβ-40 production.

FIG. 8B is a list of compounds represented in FIG. 8A.

FIGS. 9-11 show compounds useful in the methods and compositions described herein.

FIGS. 12-14 are bar graphs showing the effect of various compounds on Aβ1-40, Aβ1-42 and total (Aβ1-40 plus Aβ1-42) β-amyloid production.

FIG. 15 is a bar graph showing the effect of various compounds on β-amyloid production.

FIGS. 16-21 show compounds useful in the methods and compositions disclosed herein.

FIGS. 22A, 22B, 23A and 23B are graphs showing the effect of various compounds on Aβ1-40 and Aβ1-42 production.

FIG. 24 is a bar graph showing the effect of various compounds on Aβ1-40 production.

### DETAILED DESCRIPTION

It has been surprisingly discovered that compounds which decrease capacitative calcium entry in mammalian cells, for example, cells that overexpress amyloid precursor protein (APP) or a fragment thereof, also can decrease β-amyloid production in the mammalian cells and can be used in the diagnosis and treatment of diseases associated with the accumulation of β-amyloid in individuals. Compounds and pharmaceutical compositions comprising the compounds, are provided, that can be used in one embodiment to treat the inexorable progression of brain degeneration that is a hallmark of certain diseases associated with cerebral accumulation of Alzheimer's amyloid, such as Alzheimer's disease (AD), in animals and humans.

### DEFINITIONS

As used herein, the term “Alzheimer's amyloid” is defined as a β-amyloid amino acid fragment that is for example proteolytically derived from amyloid precursor protein (APP). A β-amyloid amino acid fragment may include, for example, about 5 to 43 or 5 to 47 consecutive amino acids of the β-amyloid sequence. As used herein, the terms “β-amyloid,” “β-amyloid protein” and “Aβ” are used interchangeably with Alzheimer's amyloid that accumulates cerebrally in an animal or human.

As used herein the phrase a cell that “overexpresses APP or fragment thereof” refers to a cell that overexpresses an amyloid precursor protein, or fragment thereof, that in one preferred embodiment, includes a β-amyloid sequence and β and γ secretase cleavage sites. The cell that overexpresses APP or a fragment thereof preferably expresses an APP or fragment thereof that produces β-amyloid in the cell in which it is expressed.

As used herein, the term “amyloidogenic disease” includes a disease associated with cerebral accumulation of Alzheimer's amyloid.

The term “alkyl”, as used herein, unless otherwise specified, includes a saturated straight, branched, or cyclic, primary, secondary, or tertiary hydrocarbon, of C₁₋₂₂ and specifically includes methyl, ethyl, propyl, isopropyl, cyclopropyl, butyl, isobutyl, secbutyl, t-butyl, pentyl, cyclopentyl, isopentyl, neopentyl, hexyl, isohexyl, cyclohexyl, cyclohexylmethyl, heptyl, cycloheptyl, octyl, cyclo-octyl, dodecyl, tridecyl, pentadecyl, icosyl, hemicosyl, and decosyl. The alkyl group may be optionally substituted with, e.g., halogen (fluoro, chloro, bromo or iodo), hydroxy, amino, alkylamino, arylamino, alkoxy, aryloxy, nitro, cyano, sulfonic acid, sulfate, heterocycle, phenyl, aryl, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “lower alkyl”, as used herein, and unless otherwise specified, includes a C₁ to C₄ saturated straight, branched, or if appropriate, a cyclic (for example, cyclopropyl) alkyl group, which is optionally substituted.

The term “aralkyl” as used herein unless otherwise specified, includes an aryl group linked to the molecule through an alkyl group.

The term “alkaryl” as used herein unless otherwise specified, includes an alkyl group linked to the molecule through an aryl group.

The term “aryl ether” as herein unless otherwise specified, includes an aryl group linked to the molecule through an ether group.

The term “alkyl ether” as herein unless otherwise specified, includes an alkyl group linked to the molecule through an ether group.

The term “aryl thioether” as herein unless otherwise specified, includes an aryl group linked to the molecule through a sulfur.

The term “alkyl thioether” as herein unless otherwise specified, includes an alkyl group linked to the molecule through a sulfur.

The term “amino” includes an “—N(R)₂” group, and includes primary amines, and secondary and tertiary amines which is optionally substituted for example with alkyl, aryl, hetercycle, and or sulfonyl groups. Thus, (R)₂ may include, but is not limited to, two hydrogens, a hydrogen and an alkyl, a hydrogen and an aryl, a hydrogen and an alkenyl, two alkyls, two aryls, two alkenyls, one alkyl and one alkenyl, one alkyl and one aryl, or one aryl and one alkenyl.

Whenever a range of carbon atoms is referred to, it includes independently and separately every member of the range. As a nonlimiting example, the term “C₁-C₁₀ alkyl” is considered to include, independently, each member of the group, such that, for example, C₁-C₁₀ alkyl includes straight, branched and where appropriate cyclic C₁, C₂, C₃, C₄, C₅, C₆, C₇, C₈, C₉ and C₁₀ alkyl functionalities.

The term “amido” includes a moiety represented by the structure “—C(O)N(R)₂”, wherein R may independently include H, alkyl, alkenyl and aryl that is optionally substituted.

The term “protected” as used herein and unless otherwise defined includes a group that is added to an atom such as an oxygen, nitrogen, or phosphorus atom to prevent its further reaction or for other purposes. A wide variety of oxygen and nitrogen protecting groups are known to those skilled in the art of organic synthesis.

The term “aryl”, as used herein, and unless otherwise specified, includes a stable monocyclic, bicyclic, or tricyclic carbon ring with up to 8 members in each ring, and at least one ring being aromatic. Examples include, but are not limited to, benzyl, phenyl, biphenyl, or naphthyl. The aryl group can be substituted with one or more moieties including halogen (fluoro, chloro, bromo or iodo), hydroxy, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term “halo”, as used herein, includes chloro, bromo, iodo, and fluoro.

The term “alkenyl” includes a straight, branched, or cyclic hydrocarbon of C2-22 with at least one double bond. Examples include, but are not limited to, vinyl, allyl, and methyl-vinyl. The alkenyl group can be optionally substituted in the same manner as described above for the alkyl groups.

The term “alkynyl” includes a C2-22 straight or branched hydrocarbon with at least one triple bond. The alkynyl group can be optionally substituted in the same manner as described above for the alkyl groups.

The term “alkoxy” includes a moiety of the structure —O-alkyl.

The term “heterocycle” or “heterocyclic” includes a saturated, unsaturated, or aromatic stable 5 to 7 membered monocyclic or 8 to 11 membered bicyclic heterocyclic ring that consists of carbon atoms and from one to three heteroatoms including but not limited to O, S, N, and P; and wherein the nitrogen and sulfur heteroatoms may optionally be oxidized, and/or the nitrogen atoms quarternized and including any bicyclic group in which any of the above-defined heterocyclic rings is fused to a benzene ring. The heterocyclic ring may be attached at any heteroatom or carbon atom which results in the creation of a stable structure. Nonlimiting examples or heterocyclic groups include pyrrolyl, pyrimidyl, pyridinyl, imidazolyl, pyridyl, furanyl, pyrazole, oxazolyl, oxirane, isooxazolyl, indolyl, isoindolyl, thiazolyl, isothiazolyl, quinolyl, tetrazolyl, bonzofuranyl, thiophrene, piperazine, and pyrrolidine.

The term “acyl” includes a group of the formula R′C(O), wherein R′ is a H, or a straight, branched, or cyclic, substituted or unsubstituted alkyl or aryl.

The term “host”, as used herein, unless otherwise specified, includes mammals (e.g., cats, dogs, horses, mice, etc.), humans, or other organisms in need of treatment, all of which can be treated or diagnosed using the methods described herein.

The term “treatment” as used herein includes any manner in which one or more of the symptoms of a disease or disorder are ameliorated or otherwise beneficially altered.

The term “pharmaceutically acceptable salt” as used herein, unless otherwise specified, includes those salts which are, within the scope of sound medical judgment, suitable for use in contact with the tissues of hosts without undue toxicity, irritation, allergic response and the like, and are commensurate with a reasonable benefit/risk ratio and effective for their intended use. The salts can be prepared in situ during the final isolation and purification of one or more compounds of the composition, or separately by reacting the free base function with a suitable organic acid. Non-pharmaceutically acceptable acids and bases also find use herein, as for example, in the synthesis and/or purification of the compounds of interest. Nonlimiting examples of such salts are (a) acid addition salts formed with inorganic salts (for example hydrochloric acid, hydrobromic acid, sulfuric acid, phosphoric acid, nitric acid, and the like), and salts formed with organic salts such as acetic acid, oxalic acid, tartaric acid, succinic acid, ascorbic acid, benzoic acid, tannic acid, and the like; (b) base addition salts formed with metal cations such as zinc, calcium, magnesium, aluminum, copper, nickel and the like; (c) combinations of (a) and (b).

The term “pharmaceutically acceptable esters” as used herein, unless otherwise specified, includes those esters of one or more compounds, which are, within the scope of sound medical judgment, suitable for use in contact with the tissues of hosts without undue toxicity, irritation, allergic response and the like, are commensurate with a reasonable benefit/risk ratio, and are effective for their intended use.

The term “pharmaceutically acceptable prodrugs” as used herein, unless otherwise specified, includes those prodrugs of one or more compounds of the composition which are, with the scope of sound medical judgment, suitable for use in contact with the tissues of hosts without undue toxicity, irritation, allergic response and the like, are commensurate with a reasonable benefit/risk ratio, and are effective for their intended use. Pharmaceutically acceptable prodrugs also include zwitterionic forms, where possible, of one or more compounds of the composition. The term “prodrug” includes compounds that are rapidly transformed in vivo to yield the parent compound, for example by hydrolysis in blood.

The term “enantiomerically enriched”, as used herein, refers to a compound that is a mixture of enantiomers in which one enantiomer is present in excess, and preferably present to the extent of 95% or more, and more preferably 98% or more, including 100%.

The term “optionally substituted,” as used herein, includes substituted and unsubstituted. Wherein a group is referenced as “optionally substituted” the group may be optionally substituted with e.g., halogen, hydroxyl, amino, alkylester, arylester, silylester, alkylamino, arylamino, alkylamido, arylamido, alkoxy, aryloxy, nitro, cyano, alkenyl, alkynyl, heterocycles, sulfonic acid, sulfate, phosphonic acid, phosphate, boronic acid, or borate.

In Vitro Assay Methods

In one embodiment, an in vitro method is provided for screening for compounds which are useful in methods of treatment and diagnosis of diseases associated with β-amyloid accumulation, wherein the method comprises detecting a reduction in CCE measurement in the cells upon exposure to the test compound in comparison to the CCE measurement in the absence of the compound. It has been discovered that such compounds that reduce CCE are useful in decreasing β-amyloid production in mammalian cells overexpressing the protein, and are therapeutically and diagnostically useful in the treatment of diseases associated with β-amyloid production, such as Azheimer's disease.

In one embodiment, the method comprises exposing cells to the test compound; measuring capacitative calcium entry (CCE) in the cells; and identifying a reduction in CCE, in comparison to control cells unexposed to the compound, as an indicator of the effectiveness of the compound in the treatment or diagnosis of a disease associated with the accumulation of β-amyloid. The cultured cells optionally are cells that overexpress amyloid precursor protein (APP) or a fragment thereof. In the assay, a measurement of CCE in cells unexposed to the compound can be obtained as a control, to allow a comparison of the CCE measurement of exposed and unexposed cells. A decrease in CCE of, for example, about 5%, 10%, 15%, 20% or more in the exposed cultured cells in comparison to cells unexposed to the compound indicates the potential therapeutic effectiveness of the compound to treat animals or humans afflicted with a disease associated with cerebral accumulation of Alzheimer's amyloid.

The CCE assay for compounds is advantageous because it is a rapid assay. High volume assays can be conducted using arrays of samples. Rapid combinatorial methods known in the art can be used, such as the use of microarrays with 1000, 10,000 or more samples with the appropriate sample delivery devices and detectors. Advantageously, the assay can be completed, e.g., in about an hour.

By way of example, in one embodiment, a 96 well plate is used. Cells are washed to remove calcium ions, e.g. with EDTA, and incubated with a fluorescent Ca²⁺ indicator, such as FluorPure, available from Molecular Probes, Eugene, Oreg. The cells are preferably washed and placed in a calcium ion free culture medium such as HBSS (Hank's balanced salt solution). A sample of cells in the culture medium and, e.g., 90 different compounds are combined in 96 wells on the plate, and control wells are included on the plate. The control is, for example, a sample of cells in culture combined with an equivalent unit volume of buffer or water as was used for the compound sample. The compound is allowed to incubate with the cells for an amount of time which can be determined with routine testing. Typically, about 15 minutes is sufficient. Baseline fluorescence measurements are taken. Thapsigargin (TG) is used to administered to deplete intracellular Ca²⁺. CaCl₂ is added in HBSS and then fluorescence is measured, as described in the Examples. The percentage of CCE inhibition is calculated as the difference between the compound treated cells and the control.

Either separately or in combination with the measurement of CCE as described above, the cells also can be tested for a reduction in β-amyloid production in cells exposed to the test compound. In the method, the concentration of β-amyloid (e.g., Aβ1-40 and/or Aβ1-42) in cells exposed to the compound can be measured and compared with a measurement of β-amyloid production in unexposed cells, for example, in a control run in parallel. A decrease in the production β-amyloid, alone or in combination, for example of about 5%, 10%, 15%, 20%, 25%, 30%, 50%, or more in the exposed cells compared to the control cells indicates the potential therapeutic effectiveness of the compound to treat animals or humans afflicted with a disease associated with cerebral accumulation of Alzheimer's amyloid. Preferably, total β-amyloid concentration (Aβ1-40+Aβ1-42) is measured. The β-amyloid is measured, e.g. in the culture medium comprising the cells, or intracellularly.

The method of measuring β-amyloid may include testing an array of compounds, e.g., in a 96 well plate, as well as one or more control samples. In the assay, the compound is often required to be incubated with the cells for about 4-48 hours, or e.g., 18-36 hours. β-amyloid can be detected using an ELISA sandwich assay using quantitatively commercially available enzymatically labeled (with horseradish peroxidase) antibodies to Aβ1-40 and Aβ1-42 as described in the Examples. The labeled antibody ELISA assay also can require on the order of 24 hours to complete. Thus, the CCE assay is advantageously less time consuming and requires less reagents than the β-amyloid assay.

CCE, also referred to as store-operated calcium influx, serves as an important calcium-refilling mechanism in both electrically non-excitable and excitable cells, such as neurons. In particular, when calcium is released from its storage sites in the endoplasmic reticulum, calcium levels rise in the cytosol, which normally is followed by calcium influx from the extracellular space that refills the cytosol and then is stored in the endoplasmic reticulum.

Measurement of CCE in cultured cells is performed using the methods for assaying CCE described herein or any method known in the art. Any appropriate assay for measuring CCE in cultured cells can be used. Skilled artisans will appreciate the experimental variability associated with various testing protocols, which typically is corrected by standardization techniques commonly known to those skilled in the art. See, e.g. Putney J. W., Jr., Sci STKE, (243):37 (2004); and Putney J. W., Jr., Mol. Interv., 1(2):84-94 (2001).

The compounds which are tested for their ability to inhibit CCE (and optionally reduce Aβ production) are screened in a range of concentrations, for example of about 1 nM to 10 mM, about 500 nM to 50 μM, or about 5 μM to 30 μM.

Cells which can be used in the assays described herein for measuring a reduction in β-amyloid production include mammalian or non-mammalian cells that overexpress APP or a fragment thereof, including but not limited to Chinese hamster ovary (CHO) cells, for example, 7W WT APP751 CHO cells. See, e.g., Koo and Squazzo, J. Biol. Chem., Vol. 269, Issue 26, 17386-17389, July 1994. Cell lines transfected with APP have been described in the art and include 7W (wt APP₇₅₁); 7W_{ΔC} (APP₇₅₁ with deletion of almost the entire cytoplasmic tail (residue 710-751); 7W_{SW} (APP₇₅₁ with the “Swedish” KM651/652NL double-mutation); and 7W_{VF} (APP₇₅₁ with the V698F mutation). See, e.g. Xia et al., Proc. Natl. Acad. Sci. USA, Vol. 94, pp. 8208-8213, July 1997; and Perez, R. & Koo, E. (1997) in Processing of the β-Amyloid Precursor Protein: Effects of C-Terminal Mutations on Amyloid Production, eds. Iqbal, K., Winblad, B., Nishimura, T., Takeda, M. & Wisniewski, H. M. (J. Wiley & Sons, London), pp. 407-416. The APP which is overexpressed can include transcripts of APP, such as, without limitation, APP751.

Cells which can be used to measure changes in CCE include most non-mammalian and mammalian cells, such as epithelial or endothelial cells, and CHO cells, and in one embodiment, 7W WT APP 751 CHO cells. Cells may be used that overexpress APP or a fragment thereof, however cells with normal expression of APP also can be used. Thus, the CCE assay is highly advantageous, since there is not a requirement for a specific cell type, or overexpression of APP. Other exemplary cells include cultured neurons, e.g., human neuronal precursor cells (HNPC), which are commercially available, for example, from QBM Cell Science (Canada); primary culture of human astrocytes; neuroblastoma cells, available e.g., from ATCC; endothelial cells, such as human brain microvascular endothelial primary culture; and human umbilical cord endothelial cells (HUVEC).

Methods of Treatment

In another embodiment, a method is provided for treating an animal or human afflicted with a disease associated with cerebral accumulation of Alzheimer's amyloid, such as Alzheimer's disease (AD), comprising administering a therapeutically effective amount of a compound disclosed herein. Administration of the compound in one embodiment results in one or more of reducing β-amyloid production, β-amyloid deposition, β-amyloid neurotoxicity (including abnormal hyperphosphorylation of tau) or microgliosis, or combination thereof. In one embodiment, the compound is one having the property of decreasing CCE, for example, by at least about 5%, 10%, 15%, 20%, or more in cells. The compound preferably has the property that it decreases CCE measured in cells, such as CHO cells, that in one embodiment overexpress APP or a fragment thereof. Alternatively, or additionally, the compound is characterized in that it reduces β-amyloid production for example by at least about 5%, 10%, 15%, 20%, 25%, 30%, 50%, or more in cells that overexpress APP or a fragment thereof, as measured, for example, in a culture medium comprising the cells or as measured intracellularly.

As used herein, reference to a compound that reduces CCE in cells, refers to a compound that reduces CCE in cells which may be 7W WT APP751 CHO cells that overexpress APP, or the cells may be selected from, e.g., cultured neurons, e.g., human neuronal precursor cells (HNPC); primary culture of human astrocytes; neuroblastoma cells, endothelial cells, such as human brain microvascular endothelial primary culture; and human umbilical cord endothelial cells (HUVEC).

As used herein, reference to a compound that reduces B-amyloid production, refers to a compound that reduces β-amyloid production in cells that overexpress APP or a fragment thereof, and the cells may be for example Chinese hamster ovary (CHO) cells that overexpress APP, for example, 7W WT APP751 CHO cells; 7W (wt APP₇₅₁) cells; 7W_{ΔC} cells; 7W_{SW} cells; or 7W_{VF} cells.

It is noted that wherever the embodiments disclosed herein refer to a reduction in β-amyloid in cells that overexpress APP, alternatively, an increase in αCTF (α C-terminal APP fragment, also known as CTF-α) and/or APPSα soluble fragment can be measured for example, in the cell culture or intracellularly, when they are produced in increased amounts from APP as the compound causes the production of β-amyloid to decrease.

It is further noted that wherever the embodiments disclosed herein refer to a reduction in β-amyloid in cells that overexpress APP, alternatively, a decrease in β CTF (β C-terminal APP fragment, also known as CTF-β) or APPSβ soluble fragment can be measured, e.g., in the cell culture media or intracellularly, when they are produced in decreased amounts from APP as the compound causes the production of β-amyloid to decrease.

In a further embodiment, a method is provided for treating animals or humans suffering from traumatic brain injury (TBI). In one embodiment, β-amyloid production, β-amyloid deposition, β-amyloid neurotoxicity (including abnormal hyperphosphorylation of tau) and/or microgliosis is reduced. The method includes administering to the animal or human, for example, immediately after the TBI, a therapeutically effective amount of a compound disclosed herein. In one embodiment, the compound is one that decreases CCE for example, by at least about 5%, 10%, 15%, 20% or more in cultured cells. The cultured cells optionally are mammalian or non-mammalian cells that overexpress APP or a fragment thereof. The method may include continuing treatment with the compound for a prescribed period of time thereafter. It has been shown that TBI increases the susceptibility to the development of AD, and thus it is believed, without being bound by the theory, that TBI accelerates brain β-amyloid accumulation and oxidative stress, which may work synergistically to promote the onset or drive the progression of AD. Alternatively or in addition to decreasing CCE in cells, the compound also may decrease β-amyloid production as disclosed herein. Treatment with the compound of animals or humans suffering from a TBI can continue, for example, for about one hour, 24 hours, a week, two weeks, 1-6 months, one year, two years or three years.

Amyloidogenic diseases which can be treated according to the methods of the present invention can include, without limitation, Alzheimer's disease, cerebral amyloid angiopathy, hereditary cerebral hemorrhage with amyloidosis Dutch-type, or other forms of familial AD and familial cerebral Alzheimer's amyloid angiopathy.

The methods of the present invention can be used on transgenic animal models for AD, such as, without limitation, PDAPP and TgAPP_{SW} mouse models, which can be useful for treating, preventing and/or inhibiting conditions associated with β-amyloid production, β-amyloid deposition, β-amyloid neurotoxicity (including abnormal hyperphosphorylation of tau) and microgliosis in the central nervous system of such animals or in humans. Transgenic animal models for AD can be constructed using standard methods known in the art, as set forth for example, without limitation, in U.S. Pat. Nos. 5,487,992; 5,464,764; 5,387,742; 5,360,735; 5,347,075; 5,298,422; 5,288,846; 5,221,778; 5,175,385; 5,175,384; 5,175,383; and 4,736,866.

Exemplary dosages of compound that can be administered include 0.001-1.0 mg/kg body weight. An exemplary dose of compound is about 1 to 50 mg/kg body weight per day, 1 to 20 mg/kg body weight per day, or 0.1 to about 100 mg per kilogram body weight of the recipient per day. Lower doses may be preferable, for example doses of 0.5-100 mg, 0.5-50 mg, 0.5-10 mg, or 0.5-5 mg per kilogram body weight per day, or e.g., 0.01-0.5 mg per kilogram body weight per day. The effective dosage range can be calculated based on the activity of the compound and other factors known in the art of pharmacology.

The compound is conveniently administered in any suitable dosage form, including but not limited to one containing 1 to 3000 mg, or 10 to 1000 mg of active ingredient per unit dosage form. An oral dosage of 50-1000 mg is possible. Lower doses may be preferable, for example from 10-100 or 1-50 mg, or 0.1-50 mg, or 0.1-20 mg or 0.01-10.0 mg. Furthermore, lower doses may be utilized in the case of administration by a non-oral route, as, for example, by injection or inhalation.

In another embodiment, the dosage can range from about 0.05 mg to 20 mg per day, from between about 2 mg to 15 mg per day, about 4 mg to 12 mg per day, and or about 8 mg per day.

In another embodiment, the dosage ranges, e.g. from about one day to twelve months, from about one week to six months, or from about two weeks to four weeks.

Because most diseases having cerebral accumulation of Alzheimer's amyloid, such as AD, are chronic, progressive, intractable brain dementias, it is contemplated that the duration of treatment with compounds disclosed herein can last for up to the lifetime of the animal or human.

Methods of Diagnosis

In still a further embodiment, a method is provided for diagnosing or determining the risk for developing a disease associated with cerebral accumulation of Alzheimer's amyloid, such as AD, in an animal or human, by taking a first measurement of β-amyloid concentration from a peripheral body fluid such as plasma, serum, whole blood, urine or cerebral spinal fluid (CSF) of the animal or human. Subsequently the method includes administering to the animal or human a diagnostically effective amount of a compound as disclosed herein. In one embodiment, the compound is one that decreases CCE in the cell, for example, by at least about 5%, 10%, 15%, 20% or more. Alternatively, or in addition to decreasing CCE, the compound decreases β amyloid production for example by at least about 5%, 10%, 15%, 20%, 25%, 30%, 50%, or more, as measured, for example, in the medium of cultured cells which overexpress APP or a fragment thereof, or as measured intracellularly. A second (selected endpoint) measurement of β-amyloid concentration is taken from plasma, serum, whole blood, urine or CSF of the animal or human at a later time, and the difference between the first measurement and the second measurement is determined. A change in the concentration of β-amyloid in plasma, serum, whole blood, urine or CSF in the second measurement compared to the first measurement indicates a risk of developing or a possible diagnosis of a disease associated with cerebral accumulation of Alzheimer's amyloid in the animal or human. In particular, an increase in peripheral β-amyloid indicates the presence of an accumulation of cerebral β-amyloid, and therefore the risk of disease or the presence of the disease.

It is believed, without being bound by any theory, that the compounds can cause an increase in β-amyloid concentration in plasma, urine, serum, whole blood or CSF by facilitating the clearance of already produced β-amyloid from the central nervous system into the periphery, thus increasing β-amyloid concentration in the peripheral fluid being assayed.

The duration of time of administration of the compound after the first peripheral body fluid measurement, up until the second (selected endpoint) peripheral body fluid measurement, is, e.g., any suitable time period, e.g. about 1-12 hours, about 1-7 days, about 14 weeks; about 2-6 months, or more. The time length can be adjusted as needed depending, for example, on the progression of the disease, and the patient. A suitable periodic (e.g., daily) dosage of the compound is administered, e.g. orally or intravenously, and the β-amyloid levels in the individual can be monitored periodically up until the endpoint. In one preferred embodiment, the compound is administered daily for about 3 days to 4 weeks from the start of administration to the endpoint measurement. The change in concentration indicative of the risk or presence of a disease associated with β-amyloid accumulation is, e.g. about 10-20% or more between the fust and endpoint measurements.

Exemplary dosages of compound that can be administered include 0.001-1.0 mg/kg body weight, for example daily. An exemplary dose of compound is about 1 to 50 mg/kg body weight per day, 1 to 20 mg/kg body weight per day, or 0.1 to about 100 mg per kilogram body weight of the recipient per day. Lower doses may be preferable, for example doses of 0.5-100 mg, 0.5-50 mg, 0.5-10 mg, or 0.5-5 mg per kilogram body weight per day, or e.g., 0.01-0.5 mg per kilogram body weight per day. The effective dosage range can be calculated based on the activity of the compound and other factors known in the art of pharmacology.

The compound is conveniently administered in any suitable dosage form, including but not limited to one containing 1 to 3000 mg, or 10 to 1000 mg of active ingredient per unit dosage form. An oral dosage of 50-1000 mg is possible. Lower doses may be preferable, for example from 10-100 or 1-50 mg, or 0.1-50 mg, or 0.1-20 mg or 0.01-10.0 mg. Furthermore, lower doses may be utilized in the case of administration by a non-oral route, as, for example, by injection or inhalation.

Compounds

A variety of compounds are provided as disclosed herein and below, which in one embodiment can be used in methods described herein, including the treatment or diagnosis of diseases associated with cerebral accumulation of Alzheimer's amyloid. In one embodiment, the compound decreases CCE, for example, by at least about 5%, 10%, 15% or 20% in cultured cells, wherein the cells optionally overexpress APP or a fragment thereof. Additionally, or alternatively, the selected compound reduces β amyloid production, for example, by at least about 5%, 10%, 15%, 20% or more, in cells that overexpress APP or a fragment thereof.

In one embodiment, a compound for the treatment and/or diagnosis of diseases associated with cerebral accumulation of Alzheimer's amyloid is provided, wherein the compound is an imidazole compound that in one embodiment decreases CCE, for example, by at least about 10% or more in cultured cells which optionally overexpress APP or a fragment thereof. In one embodiment, alternatively or in addition to decreasing CCE, the compound reduces β amyloid production, for example, by at least about 20% or more in cells that overexpress APP or fragment thereof.

In one embodiment, a compound for the treatment and/or diagnosis of diseases associated with cerebral accumulation of Alzheimer's amyloid is provided, wherein the compound is an isoquinoline alkaloid compound. The isoquinoline compound in one embodiment decreases CCE, for example, by at least about 10% or more in cultured cells that optionally are cells that overexpress APP or a fragment thereof. In one embodiment, alternatively or in addition to decreasing CCE, the compound reduces β amyloid production, for example, by at least about 20% or more, in a cell that overexpress APP or fragment thereof, as measured intracellularly or extracellularly.

In one embodiment, a compound for the treatment and/or diagnosis of diseases associated with cerebral accumulation of Alzheimer's amyloid is provided, wherein the compound is an calmodulin-mediated enzyme activation inhibitor that in one embodiment decreases CCE, for example, by at least about 10% or more in cultured cells that optionally are cells that overexpress APP or a fragment thereof. In one embodiment, alternatively or in addition to decreasing CCE, the compound reduces β amyloid production, for example, by at least about 20% or more in cells that overexpress APP or a fragment thereof, as measured intracellularly or extracellularly.

In one embodiment, a compound for the treatment and/or diagnosis of diseases associated with cerebral accumulation of Alzheimer's amyloid is provided, wherein the compound is an inhibitor of kinase activity of the platelet-derived growth factor (PDGF) receptor, and wherein the compound in one embodiment decreases CCE, for example, by at least about 10% or more in cultured cells that in one embodiment are cells that overexpress APP or a fragment thereof. Optionally, the compound is one that additionally or alternatively reduces β amyloid production, for example, by at least about 20% or more in cells that overexpress APP or a fragment thereof.

In one embodiment, a compound for the treatment and/or diagnosis of diseases associated with cerebral accumulation of Alzheimer's amyloid is provided, wherein the compound is an NF-kB activation inhibitor that in one embodiment decreases CCE, for example, by at least about 10% or more in cultured cells which optionally are cells that overexpress APP or a fragment thereof. The compound optionally, in addition to or alternatively, reduces β amyloid production, for example, by at least about 20% or more, in cells that overexpress APP or a fragment thereof.

In one embodiment, a compound for the treatment and/or diagnosis of diseases associated with cerebral accumulation of Alzheimer's amyloid is provided, wherein the compound is a diterpene or triterpene compound that in one embodiment decreases CCE, for example, by at least about 10% or more in cultured cells that in one embodiment are cells that overexpress APP or a fragment thereof. Optionally, the compound is one that additionally or alternatively reduces β amyloid production, for example, by at least about 20% or more, in cells that overexpress APP or a fragment thereof.

In one embodiment, a compound for the treatment and/or diagnosis of diseases associated with cerebral accumulation of Alzheimer's amyloid is provided, wherein the compound is a quinazoline compound, and wherein in one embodiment the compound decreases CCE, for example, by at least about 10% or more in cultured cells that in one embodiment are cells that overexpress APP or a fragment thereof. Optionally, the compound is one that additionally or alternatively reduces β amyloid production, for example, by at least about 20% or more in cells that overexpress APP or a fragment thereof.

In one embodiment, a compound for the treatment and/or diagnosis of diseases associated with cerebral accumulation of Alzheimer's amyloid is provided, wherein the compound is a sesquiterpene lactone that in one embodiment decreases CCE, for example, by at least about 10% or more in cultured cells that in one embodiment are cells that overexpress APP or a fragment thereof. Optionally, the compound is one that additionally or alternatively reduces β amyloid production, for example, by at least about 20% or more, in cells that overexpress APP or a fragment thereof.

In one embodiment, a compound for the treatment and/or diagnosis of diseases associated with cerebral accumulation of Alzheimer's amyloid is provided, wherein the compound is an inhibitor of IkappaB kinase 2 (IKK-2), and wherein the compound in one embodiment decreases CCE, for example, by at least about 10% or more in cultured cells that in one embodiment are cells that overexpress APP or a fragment thereof. Optionally, the compound is a compound that additionally or alternatively to decreasing CCE, reduces β amyloid production, for example, by at least about 20% or more, in cells that overexpress APP or a fragment thereof.

In one embodiment, the compound is a compound of Formula I, or a salt, ester or prodrug thereof, including R and S isomers thereof, wherein: 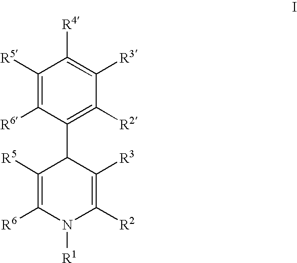
R¹ is H, alkyl (including straight chain, branched, and cyclic alkyl), optionally substituted aryl, optionally substituted heterocycle, allyl or aryl ether;
R² and R⁶ are independently alkyl, alkyl ether, aryl ether, halogen, or hydroxy;
R³ and R⁵ are independently optionally substituted alkyl ester, aryl ester, silyl ester, alkyl amide, aryl amide, cyano, or nitro;
R^{2′} and R^{6′} are independently H, alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle;
R^{3′} and R^{5′} are independently H, alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle;
R^{4′} is independently H, alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle;
alternatively, R^{2′} and R^{3′} together can optionally form a 4, 5, 6 or 7 membered heterocycle containing 1, 2, or 3 heteratoms and can be optionally substituted with alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle;
alternatively, R^{3′} and R^{4′} together can optionally form a 4, 5, 6 or 7 membered heterocycle containing 1, 2, or 3 heteratoms and can be optionally substituted with alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle;
alternatively, R^{4′} and R^{5′} together can optionally form a 4, 5, 6 or 7 membered heterocycle containing 1, 2, or 3 heteratoms and can be optionally substituted with alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle;
alternatively, R^{5′} and R^{6′} together can optionally form a 4, 5, 6 or 7 membered heterocycle containing 1, 2, or 3 heteratoms and can be optionally substituted with alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle.

In one embodiment, the compound is a compound of Formula I, or a salt, ester or prodrug thereof, including R and S isomers thereof, wherein: 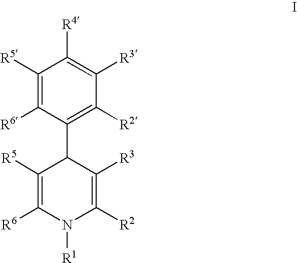
R¹ is H, alkyl (including straight chain, branched, and cyclic alkyl), optionally substituted aryl, optionally substituted heterocycle, alkyl or aryl ether;
R² and R⁶ are independently alkyl, alkyl ether, aryl ether, halogen, or hydroxy;
R³ and R⁵ are independently alkyl ester, aryl ester, silyl ester, alkyl amide, aryl amide, cyano, or nitro;
R^{2′} and R^{6′} are independently H, optionally substituted alkyl, alkyl ether, aryl ether, halogen, hydroxy, nitro, or optionally substituted heterocycle;
R^{3′} and R^{5′} are independently H, optionally substituted alkyl, alkyl ether, aryl ether, halogen, hydroxy, nitro, or optionally substituted heterocycle;
R^{4′} is independently H, alkyl, alkyl ether, aryl ether, halogen, hydroxy, nitro, or optionally substituted heterocycle.

In one embodiment, the compound comprises at least two nitro substituents.

In one embodiment, R³═R⁵ and R³=alkyl ester, wherein the alkyl is optionally substituted with a group other than alkoxyl.

In one embodiment, R³═R⁵ and R³=alkyl ester, wherein the alkyl is optionally substituted.

In one embodiment, R³═R⁵ and R³ is unsubstituted alkyl ester.

In another embodiment of a compound of Formula I or a salt, ester or prodrug thereof, including an R or S isomer thereof, wherein:
R¹ is H, alkyl (including straight chain, branched, and cyclic alkyl), optionally substituted aryl, optionally substituted heterocycle, alkyl or aryl ether;
R² and R⁶ are independently allyl, alkyl ether, aryl ether, halogen, or hydroxy;
R³ and R⁵ are independently alkyl ester, aryl ester, silyl ester, alkyl amide, aryl amide, cyano, or nitro;
R^{2′} and R^{6′} are independently H, alkyl, alkyl ether, aryl ether, halogen, hydroxy, nitro, or optionally substituted heterocycle;
R^{3′} and R^{5′} are independently H, optionally substituted alkyl, alkyl ether, aryl ether, halogen, hydroxy, or optionally substituted heterocycle; and
R^{4′} is independently H, optionally substituted alkyl, alkyl ether, aryl ether, halogen, hydroxy, nitro, or optionally substituted heterocycle.

In another embodiment of a compound of Formula I or a salt, ester or prodrug thereof, including an R or S isomer thereof, wherein:
R¹ is H, alkyl (including straight chain, branched, and cyclic alkyl), optionally substituted aryl, optionally substituted heterocycle, or alkyl;
R² and R⁶ are independently alkyl, alkyl ether, aryl ether, halogen, or hydroxy;
R³ and R⁵ are independently alkyl ester, aryl ester, silyl ester, alkyl amide, aryl amide, cyano, or nitro;
R^{2′} and R^{6′} are independently H, alkyl, alkyl ether, aryl ether, halogen, hydroxy, nitro, or optionally substituted heterocycle;
R^{3′} and R^{5′} are independently H, optionally substituted alkyl, alkyl ether, aryl ether, halogen, hydroxy, or optionally substituted heterocycle; and
R^{4′} is independently H, optionally substituted alkyl, alkyl ether, aryl ether, halogen, hydroxy, nitro, or optionally substituted heterocycle.

In another embodiment, the compound is a compound of Formula I, or a salt, ester or prodrug there of, including an R or S isomer thereof, wherein:
R¹ is H, alkyl including straight chain, e.g., methyl; branched alkyl, e.g., isopropyl; cyclic alkyl, e.g., cyclohexyl; substituted aryl, e.g., o-chlorophenyl; substituted heterocycle, e.g., 2-methyl furyl; alkyl ether, e.g., methoxy; or aryl ether, e.g., phenoxy;
R²═R⁶ and each are alkyl, e.g. methyl; alkyl ether, e.g., ethoxy; or halogen, e.g., F;
R³═R⁵ and each are alkyl ester, e.g., ethyl ester; aryl ester, e.g., benzoate; silyl ester; alkyl amide, e.g., methyl amide; aryl amide, e.g., phenyl amide; cyano; or nitro;
R^{2′} and R^{6′} are independently H, alkyl, e.g. methyl; alkyl ether e.g. ethoxy; aryl ether e.g. phenoxy; halogen, e.g. F; hydroxy; nitro; or heterocycle, e.g., 2-methyl furyl;
R^{3′} and R^{5′} are independently H, alkyl, e.g., methyl; alkyl ether, e.g. ethoxy; aryl ether, e.g., phenoxy; halogen, e.g., F; hydroxy; nitro; or heterocycle, e.g., 2-methyl furyl; and
R^{4′} is H, alkyl, e.g., methyl; alkyl ether, e.g. ethoxy; aryl ether, e.g., phenoxy, halogen, e.g., F; hydroxy; nitro; or heterocycle, e.g., 2-methyl furyl.

In one embodiment, the compound is a compound of Formula I, or a salt, ester or prodrug thereof, including R and S isomers thereof, wherein:
R¹ is H;
R² and R⁶ are independently alkyl, e.g. methyl or ethyl;
R³ and R⁵ are independently cyano or alkyl ester;
R^{2′} and R^{6′} are independently H, halo, or nitro;
R^{3′} and R^{5′} are independently H or halo; and
R^{4′} is independently H, alkyl, alkyl ether, halo, or nitro.

In another embodiment, the compound is a compound of Formula I, or a salt, ester or prodrug thereof, including R and S isomers thereof, wherein:
R¹ is H;
R² and R⁶ are independently alkyl;
R³ and R⁵ are independently alkyl ester, wherein, in at least one of R₂ and R₃ the alkyl of the alkyl ester comprises at least 10, 20 or 30 carbon atoms, e.g. 10 to 30 carbon atoms;
R^{2′}, R^{3′}, R^{4′}, R^{5′}, and R^{6′} are independently H, halo, or nitro.

In another embodiment, the compound is a compound of Formula I, or a salt, ester or prodrug thereof, including R and S isomers thereof, wherein:
R¹ is H;
R² and R⁶ each are alkyl, e.g. methyl;
R³ and R⁵ are independently C(O)OCH₂CH₂Oalkyl, wherein the alkyl is, e.g. methyl and is optionally substituted;
R^{2′}, R^{3′}, R^{4′}, R^{5′}, and R^{6′} are independently H, halo, or nitro.

In another embodiment, the compound is a compound of Formula I, or a salt, ester or prodrug thereof, including R and S isomers thereof, wherein:
R¹ is H;
R² and R⁶ each are alkyl, e.g. methyl;
R³ and R⁵ are independently C(O)Oalkyl, wherein the alkyl is substituted with alkenyl or alkynyl, e.g. R³and R⁵ are C(O)OCH₂CHCH₂;
R^{2′}, R^{3′}, R^{4′}, R^{5′}, and R^{6′} are independently H, halo, or nitro.

In another embodiment, the compound is a compound of Formula I, or a salt, ester or prodrug thereof, including R and S isomers thereof, wherein:
R¹ is H;
R² and R⁶ each are CH₂Oalkyl, e.g. CH₂OCH₃;
R³ and R⁵ are independently C(O)Oalkyl, e.g. C(O)OCH₃;
R^{2′}, R^{3′}, R^{4′}, R^{5′}, and R^{6′} are independently H, halo, or nitro.

In another embodiment, the compound is a compound of Formula I, or a salt, ester or prodrug thereof, including R and S isomers thereof, wherein:
R¹ is H;
R² and R⁶ each are alkyl, e.g. methyl;
R³ and R⁵ are independently C(O)Oalkyl, e.g. C(O)OCH₂CH₃, or C(O)OCH₂C(CH₃)₃;
R^{2′} and R^{6′} are independently H, F, Br, or nitro.
R^{3′} and R^{5′} each are H.
R^{4′} is H or halo.

In another embodiment, the compound is a compound of Formula I, or a salt, ester or prodrug thereof, including R and S isomers thereof, wherein:
R¹ is H;
R² and R⁶ each are alkyl, e.g. methyl;
R³ and R⁵ are independently C(O)Oalkyl, e.g. C(O)OCH₂CH₃, or C(O)OCH₂C(CH₃)₃;
R^{2′} and R^{6′} each are H or F and not the same;
R^{3′}, R^{4′}, R^{5′} are independently H, or Br.

In another embodiment, the compound useful in the methods and compositions disclosed herein is a compound of formula II, or a salt, ester or prodrug there of, including an R or S isomer thereof, wherein: 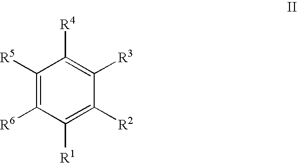
R¹ is heterocycle, optionally substituted with one or more of alkyl, alkyl ether, aryl ether, alkylaryl, arylalkyl, halogen, hydroxy, optionally substituted alkyl ester, optionally substituted aryl ester, alkyl amide, aryl amide, or nitro;
R² and R⁶ are independently optionally substituted alkyl, heteroalkyl, alkyl ether, aryl ether, halogen, hydroxy, nitro, cyano, or heterocycle; and
R³ and R⁵ are independently H, alkyl, alkyl ether, aryl ether, halogen, hydroxy, nitro, or heterocycle;
R⁴ is H, alkyl, alkyl ether, aryl ether, halogen, hydroxy, nitro, cyano, or heterocycle;
   wherein, in one embodiment, at least two of R¹, R², R³, R⁴, R⁵ and R⁶ are nitro.

In another embodiment, the compound is a compound of formula II, or a salt, ester or prodrug there of, including an R or S isomer thereof, wherein:
R¹ is heterocycle, optionally substituted with one or more of alkyl, alkyl ether, aryl ether, alkylaryl, arylalkyl, halogen, hydroxy, optionally substituted alkyl ester, optionally substituted aryl ester, alkyl amide, aryl amide;
R² and R⁶ are independently optionally substituted alkyl, heteroalkyl, alkyl ether, aryl ether, halogen, hydroxy, cyano, or heterocycle;
R³ and R⁵ are independently H, alkyl, alkyl ether, aryl ether, halogen, hydroxy, or heterocycle; and
R⁴ is H, alkyl, alkyl ether, aryl ether, halogen, hydroxy, nitro, cyano, or heterocycle.

In another embodiment, the compound is a compound of formula II, or a salt, ester or prodrug there of, including an R or S isomer thereof, wherein:
R¹ is unsubstituted heterocycle, e.g., furyl, or is optionally heterocycle substituted with alkyl, e.g., methyl; alkyl ether, e.g. methoxy; aryl ether, e.g. phenoxy; halogen, e.g., F; hydroxy; alkyl ester, e.g. ethyl ester; aryl ester, e.g. benzoate; alkyl amide, e.g., methyl amide; aryl amide, e.g., phenyl amide; nitro; or cyano;
R²═R⁶ and are each H, optionally substituted alkyl, e.g. methyl; alkyl ether, e.g. methoxy; aryl ether, e.g., phenoxy; halogen, e.g., F; hydroxy; nitro; cyano; or heterocycle, e.g., pyrazole;
R³═R⁵ and are each H, alkyl, e.g., methyl; alkyl ether, e.g., methoxy; aryl ether, e.g., phenoxy; halogen, e.g., F; hydroxy; nitro; cyano, or heterocycle, e.g., pyrazole;
R⁴ is H, alkyl, e.g., methyl; alkyl ether, e.g., methoxy; aryl ether, e.g., phenoxy; halogen, e.g., F; hydroxy; nitro; cyano; or heterocycle, e.g. pyrazole.

In another embodiment, the compound useful in the methods and compositions disclosed herein is a compound of formula III, or a salt, ester or prodrug there of, including an R or S isomer thereof, wherein: 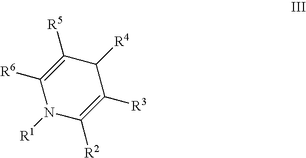
R¹ is alkyl including straight chain, branched, or cyclic alkyl; optionally substituted aryl; optionally substituted heterocycle; alkyl; aryl ether; or aryl-O-(optionally substituted aryl);
R² and R⁶ are independently alkyl, alkyl ether, aryl ether, halogen, or hydroxy;
R³ and R⁵ are independently alkyl ester, aryl ester, silyl ester, alkyl amide, aryl amide, cyano, or nitro;
R⁴ is alkyl (including straight chain, branched, and cyclic) or heterocycle optionally substituted e.g. with one or more of alkyl, alkyl ether, aryl ether, halogen, hydroxy, alkyl ester, aryl ester, alkyl amide, aryl amide, or nitro;

In another embodiment of the compound of formula III, or a salt, ester or prodrug there of, including an R or S isomer thereof, wherein:
R¹ is H, alkyl (including straight chain, e.g., methyl; branched, e.g. isopropyl; cyclic, e.g. cyclohexyl); optionally substituted aryl, e.g., o-chlorophenyl; substituted heterocycle (substituted at one or more positions by alkyl, e.g., methyl; alkyl ether, e.g., methoxy; aryl ether, e.g., phenoxy; halogen, e.g. F; hydroxy; alkyl ester, e.g., ethyl; aryl ester, e.g., benzoate; alkyl amide, e.g., methyl amide; aryl amide, e.g., phenyl amide; nitro; or cyano) unsubstituted heterocycle, e.g., furyl; alkyl ether, e.g., methoxy; or aryl ether, e.g., phenoxy;
R²═R⁶ and each are alkyl, e.g., methyl; alkyl ether, e.g. ethoxy; halogen, e.g., F; or hydroxy;
R³═R⁵ and each are alkyl ester, e.g., ethyl; aryl ester, e.g., benzoate; silyl ester; alkyl amide, e.g. methyl; aryl amide, e.g., phenyl; cyano; or nitro; and
R⁴ is alkyl (including straight chain, e.g., methyl; branched, e.g., isopropyl; cyclic e.g., cyclohexyl); optionally substituted aryl, e.g., o-chlorophenyl; substituted heterocycle (substituted at one or more positions by alkyl, e.g., methyl; alkyl ether, e.g., methoxy; aryl ether, e.g., phenoxy; halogen, e.g., F; hydroxy; alkyl ester, e.g., ethyl ester; aryl ester, e.g., benzoate; alkyl amide e.g. methyl amide; aryl amide, e.g., phenyl amide; nitro; or cyano); or unsubstituted heterocycle, e.g. furyl.

In another embodiment, the compound useful in the methods and compositions disclosed herein is a compound of formula IV, or a salt, ester or prodrug there of, including an R or S isomer thereof, wherein: 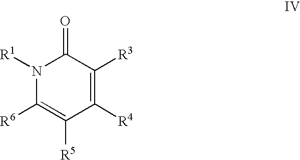
R¹ is H, alkyl (including straight chain, branched, and cyclic); optionally substituted aryl; or optionally substituted heterocycle;
R³ is cyano, nitro, alkyl ester, aryl ester, silyl ester, alkyl amide, or aryl amide;
R⁴ is alkyl, haloalkyl, cyano, unsubstituted aryl, substituted aryl (substituted at one more positions by, e.g., cyano, nitro, halo, ester, carboxylic or carbonyl); unsubstituted heterocycle, substituted heterocycle (substituted at one more positions by e.g. alkyl, alkyl ether, aryl, aryl ether, halogen, hydroxy, ester, alkyl ester, aryl ester, alkyl amide, aryl amide, nitro, or cyano); and
R⁵ and R⁶ each are independently H, alkyl ester, aryl ester, silyl ester, alkyl amide, aryl amide, cyano, nitro, alkyl ether, aryl ether, halogen, hydroxy, alkyl (including straight chain, branched, and cyclic), or optionally substituted aryl.

In another embodiment, the compound is a compound of formula IV, or a salt, ester or prodrug there of, including an R or S isomer thereof wherein:
R¹ is H, alkyl (including straight chain, e.g., methyl; branched, e.g., isopropyl; cyclic, e.g., cyclohexyl; substituted aryl, e.g., o-chlorophenyl); substituted heterocycle (substituted at one or more positions by alkyl, e.g., methyl; alkyl ether, e.g., methoxy; aryl ether, e.g., phenoxy; halogen, e.g., F; hydroxy; alkyl ester, e.g., ethyl; aryl ester, e.g., benzoate; alkyl amide, e.g., methyl; aryl amide, e.g., phenyl; nitro, or cyano) or unsubstituted heterocycle, e.g., furyl;
R³═R⁵═R⁶ and are H, alkyl ester, e.g., ethyl; aryl ester, e.g., benzoate; silyl ester; alkyl amide, e.g., methyl amide; aryl amide, e.g., phenyl amide; cyano; nitro; alkyl ether, e.g., methoxy; and aryl ether, e.g., phenoxy; halogen, e.g., F; hydroxy; alkyl (including straight chain, e.g., methyl; branched, e.g., isopropyl; and cyclic, e.g., cyclohexyl); optionally substituted aryl, e.g., o-chlorophenyl; or unsubstituted aryl, e.g., naphthyl; and
R⁴ is substituted heterocycle (substituted at one or more positions by alkyl, e.g., methyl; alkyl ether, e.g., methoxy; aryl; aryl ether, e.g., phenoxy; halogen, e.g., F; hydroxy; alkyl ester, e.g., ethyl ester; aryl ester, e.g., benzoate; alkyl amide, e.g., methyl amide; aryl amide, e.g. phenyl amide; nitro; or cyano) or unsubstituted heterocycle, e.g., furyl.

In another embodiment, the compound useful in the methods and compositions disclosed herein is a compound of formula V, or a salt, ester or prodrug there of, including an R or S isomer thereof, wherein: 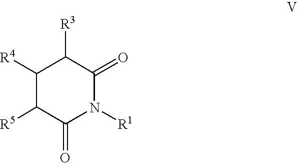
R¹ is substituted or unsubstituted aryl, alkyl, alkyl ether, substituted or unsubstituted aryl ether (e.g., 4(4-chlorophenoxy)phenyl), substituted heterocycle (substituted at different positions by alkyl, alkyl ether, aryl ether, halogen, hydroxy, alkyl ester, aryl ester, alkyl amide, aryl amide, nitro, or cyano), unsubstituted heterocycle, or halogen;
R³, R⁴ and R⁵ are independently H, alkyl ester, aryl ester, silyl ester, alkyl amide, aryl amide, cyano, nitro, alkyl ether, aryl ether, halogen, hydroxy, alkyl (including straight chain, branched, or cyclic), substituted aryl, unsubstituted aryl, or heterocycle.

In another embodiment, the compound is a compound of formula V, or a salt, ester or prodrug there of, including an R or S isomer thereof wherein:
R¹ is substituted aryl, e.g., o-chlorophenyl; unsubstituted aryl, e.g., naphthyl; alkyl, e.g., methyl; alkyl ether, e.g., methoxy; substituted aryl ether, e.g., 4(4-chlorophenoxy)phenyl; unsubstituted aryl ether, e.g., phenoxyphenyl; substituted (at one or more positions by alkyl, e.g., methyl; alkyl ether, e.g., methoxy; aryl ether, e.g., phenoxy; halogen, e.g., F; hydroxy; alkyl ester, e.g., ethyl; aryl ester, e.g., benzoate; alkyl amide, e.g., methyl amide; aryl amide, e.g., phenyl amide; nitro; or cyano) or unsubstituted heterocycle, e.g., piperidine; and
R³═R⁴═R⁵ and each are H, alkyl ester, e.g., ethyl; aryl ester, e.g., benzoate; silyl ester; alkyl amide, e.g., methyl amide; aryl amide, e.g., phenyl amide; cyano; nitro; alkyl ether, e.g., methoxy; aryl ether, e.g., phenoxy; halogen, e.g., F; hydroxy; alkyl (including straight chain, e.g., methyl; branched, e.g., isopropyl; and cyclic, e.g., cyclohexyl); substituted aryl, e.g., o-chlorophenyl; or unsubstituted aryl, e.g., phenyl.

In another embodiment of a compound of Formula V:
R¹ is halo substituted phenoxyphenyl;
R³═R⁵═H; and
R⁴ is optionally substituted phenyl, substituted e.g. with OH or halo.

In another embodiment, the compound useful in the methods and compositions disclosed herein is a compound of formula VI, or a salt, ester or prodrug there of, including an R or S isomer thereof, wherein: 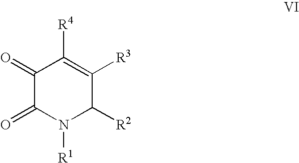
R¹ and R³ are independently alkyl ether, aryl ether, halogen, hydroxy, alkyl (including straight chain, branched, or cyclic), substituted aryl or unsubstituted aryl; and
R² and R⁴ are independently H, alkyl ether, substituted and unsubstituted aryl ether, substituted heterocycle (substituted at one or more positions,e.g., by alkyl, alkyl ether, aryl ether, halogen, hydroxy, alkyl ester, aryl ester, alkyl amide, aryl amide, nitro, or cyano), unsubstituted heterocycle, halogen, hydroxy, alkyl ester, aryl ester, silyl ester, alkyl amide, aryl amide, cyano, or nitro.

In another embodiment, the compound a compound of formula VI, or a salt, ester or prodrug there of, including an R or S isomer thereof wherein:
R¹═R³ and is alkyl ether, e.g., methoxy; substituted aryl ether, e.g., 4(4-chlorophenoxy)phenyl; unsubstituted aryl ether, e.g., methoxy phenyl; halogen, e.g., F; hydroxy; alkyl, including straight chain, e.g., methyl; branched, e.g., isopropyl; or cyclic, e.g., cyclohexyl); substituted aryl, e.g., o-chlorophenyl; or unsubstituted aryl, e.g., phenyl; and
R²═R⁴ is H, alkyl ether, e.g., methoxy; substituted aryl ether, e.g., 4(4-chlorophenoxy)phenyl; unsubstituted aryl ether, e.g., methoxy phenyl; substituted heterocycle (substituted, e.g., at one or more positions by alkyl, e.g., methyl; alkyl ether, e.g., methoxy; aryl ether, e.g., phenoxy; halogen, e.g., F; hydroxy; alkyl ester, e.g., ethyl; aryl ester, e.g., benzoate; alkyl amide, e.g., methyl; aryl amide, e.g., phenyl; nitro; or cyano); unsubstituted heterocycle, e.g., pyrazole; halogen, e.g., F;

hydroxy; alkyl ester, e.g., ethyl; aryl ester, e.g., benzoate; silyl ester; alkyl amide, e.g., methyl amide; aryl amide, e.g., phenyl amide; cyano; or nitro.

In another embodiment of the compound of Formula VI, or a salt, ester or prodrug there of, including an R or S isomer thereof wherein:
R¹ is alkyl, which in one embodiment is C3-12 alkyl, e.g., cycloalkyl, including cyclohexyl or cyclopentyl;
R² and R⁴ are independently H or halo; and
R³ is unsubstituted or substituted aryl, e.g., phenyl substituted for example with halo.

In another embodiment, a compound of Formula VII, or a salt, ester or prodrug thereof, including an R or S isomer thereof, is provided: 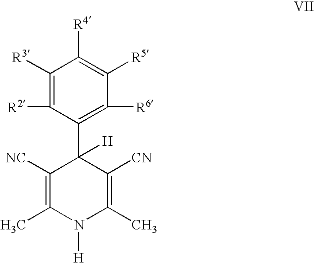
wherein:
R^{4′} is H, halo, alkyl, or aryl;
R^{3′} and R^{5′} are independently H, halo, alkyloxy, hydroxy, or aryl; and
R^{2′} and R^{6′} are independently H, halo, alkyl, or aryl.

In another embodiment, a compound of Formula VII, or a salt, ester or prodrug thereof, including an R or S isomer thereof, is provided: 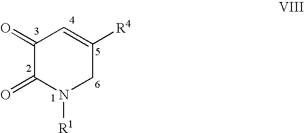
wherein:
R⁴ is optionally substituted aryl, e.g., phenyl optionally substituted with halo; and
R¹ is alkyl, e.g., cycloalkyl.

In another embodiment, the compound is a compound of Formula IX, or a prodrug, or salt thereof, including an R or S isomer: 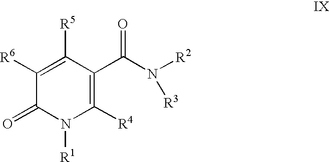
wherein:
R¹ is alkyl, hydrogen, substituted aryl (e.g., with halogen, ether, alkyl, haloalkyl, or hydroxy) or unsubstituted aryl;
R², R³, and R⁴ are independently, alkyl, haloalkyl, thioalkyl, hydroxy, hydrogen, substituted aryl (substituted e.g., with halogen, ether, haloether, alkyl, haloalkyl, or hydroxy), unsubstituted aryl, substituted heterocycle (substituted e.g., with alkyl, halogen, haloalkyl, or amide) or unsubstituted hetrocyclic;
R⁵ is alkyl, haloalkyl, hydroxy, hydrogen, ether, haloether
R⁶ is nitro, cyano, hydrogen, ester, amide, carboxylic, or carbonyl.

In another embodiment, the compound is a compound of Formula X, or a prodrug, or salt thereof, including an R or S isomer: 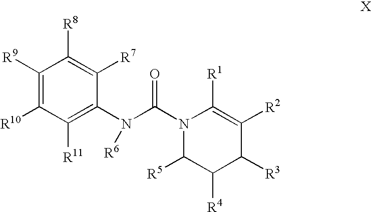
wherein R¹, R³, R⁵, R⁶, R⁷, R⁸, R⁹, R¹⁰, and R¹¹ are independently, alkyl, haloalkyl, hydroxy, hydrogen, ether, haloether, thioalkyl, halogen; and
R² and R⁴ are independently amide, ester, carboxylic, or nitro.

In another embodiment, the compound is a compound of Formula XI, or a prodrug, or salt thereof, including an R or S isomer: 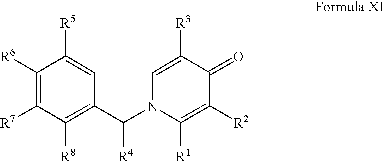
wherein R² is alkyl ester, aryl ester, alkyl amide, aryl amide, hydrogen, carboxylic, nitro, or cyano; and
R³, R¹, R⁴, R⁵, R⁶, R⁷, R⁸ are independently alkyl, haloalkyl, hydroxy, H, ether, haloether, thioalkyl, or halogen.

Other examples of compounds useful in the methods and compositions disclosed herein are listed below. In one embodiment, the compound can decrease CCE, for example, by at least about 10% or more in cells that, e.g, overexpress APP or a fragment thereof, and optionally reduce β amyloid production, for example, by at least about 20% or more, in cultured cells which overexpress APP or a fragment thereof.

SKF 96365, 1-[beta-[3-(4-methoxyphenyl)propoxy]4-methoxyphenethyl]-1H-imidazole hydrochloride:

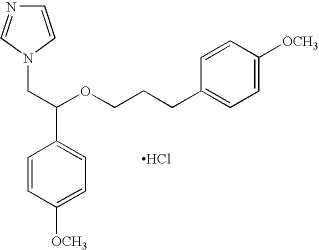

econazole, (RS)-1-[2,4-dichloro-beta-(p-chlorobenzyl-oxy)phen-ethyl]imidazole nitrate:

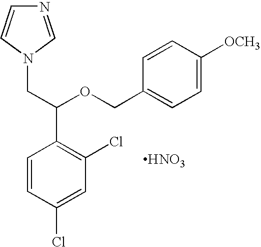

clotrirazole, 1-[(2-chlorophenyl)diphenylmethyl]-1H-imidazole (and other imidazole-based cytochrome P-450 inhibitors of divalent cation uptake that are mediated by depletion of intracellular stores induced by depletion of the intracellular calcium pool, such as by exposure to calcium-free solutions):

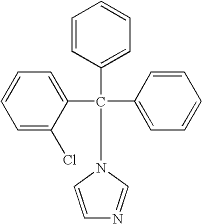

SR33805, 3,4-dimethoxy-N-methyl-N-[3-[4-[[1-methyl-3-(1-methylethyl)-1H-indol-2-yl]sulfonyl]phenoxy]propyl]benzeneethanamine oxalate, and other potent calcium antagonists that binds allosterically to the α₁-subunit of L-type calcium channels:

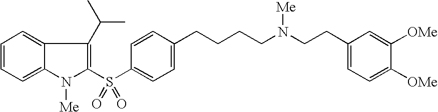

Ioperamide, 4-(4-chlorophenyl)-4-hydroxy-N,N-dimethyl-α,α-diphenyl-1-peper-idinebutanamide, a calcium channel blocker as well as an antidiarrheal agent with high affinity for both peripheral and central opioid receptors (at low micromolar concentrations), loperamide blocks broad spectrum neuronal high voltage-activated (HVA) calcium channels and at high concentrations it reduces calcium flux through N-methyl-D-aspartate (NMDA) receptor operated channels:

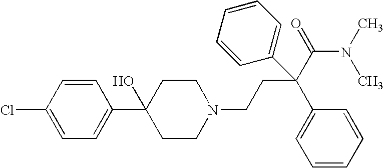

tetrandrine (Tet), a bis-benzylisoquinoline alkaloid isolated from the Chinese medicinal herb-root of Stephania tetrandra:

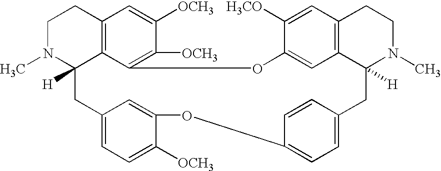

calmidazolium chloride (R24571), 1-[bis(p-chlorophenyl)methyl]-3-[2-(2,4-di-chloro-β-(2,4-dichlorobenzyl-oxy)phenethyl)]-imidazolium chloride, which binds reversibly to calmodulin, thus inhibiting calmodulin-mediated enzyme activation, and other calnodulin-mediated enzyme activation inhibitors (R24571 also blocks sodium channel and voltage-gated calcium channels, inhibits the calcium/calmodulin-induced activation of myosin light chain kinase in a concentration dependent manner, and inhibits calmodulin N-methyltransferase):

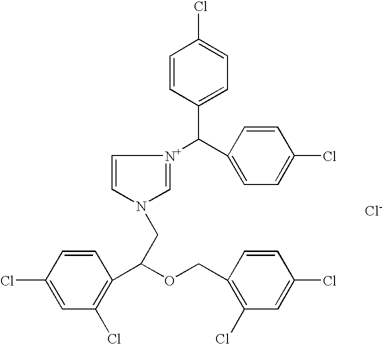

amlodipine, (R,S) 3-ethyl-5-methyl-2-(2-aminoethoxymethyl)-4-(2-chlorophenyl)-1,4-dihydro-6-methyl-3,5-pyridinedicarboxylate benzenesulfonate, a dihydropyridine calcium antagonist that inhibits the transmembrane influx of calcium ions into vascular smooth muscle and cardiac muscle (amlodipine binds to both dihydropyridine and nondihydropyridine binding sites and inhibits calcium ion influx across cell membranes selectively, having a greater effect on vascular smooth muscle cells than on cardiac muscle cells):

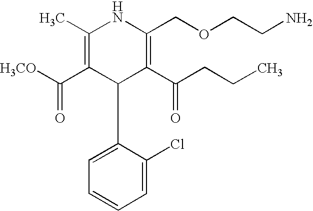

nitrendipine (1,4-Dihydro-2,6-dimethyl-4-(meta-nitrophenyl)-3,5-pyridine-dicarboxylic acid, ethyl methyl ester (ethyl methyl 1,4-dihydro-2,6-dimethyl-4-(meta-nitrophenyl)-3,5-pyridine dicarboxylate), and other dihydropyridine calcium channel blockers:

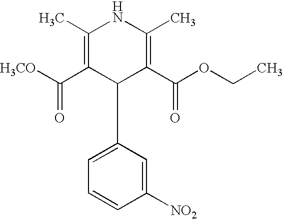

N-propargylnitrendipine (MRS 1845),1,4-dihydro-2,6-dimethyl-4-(3-nitro-phenyl)-1-(2-propynyl)-3,5-pyridinedicarboxylic acid, ethyl, methyl ester, a dihydropyridine compound calcium channel blocker:

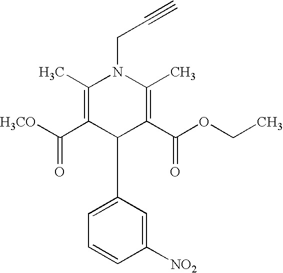

tyrphostin A9 ([[3,5-bis(1,1-dimethylethyl)-4-hydroxy-phenyl]methylene]propane-dinitrile), and other selective inhibitors of kinase activity of the platelet-derived growth factor (PDGF) receptor, or derivatives thereof:

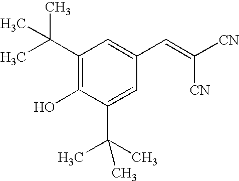

Various other dihydropyridine compounds can be used according to the treatment and diagnostic methods herein, including, without limitation, the following compounds and derivatives, salts and prodrugs thereof Particularly preferred are those compounds which that can decrease CCE, for example, by at least about 10% or more in cells overexpressing β-amyloid, and optionally may reduce β amyloid production, for example, by at least about 20% or more in the cells.

Examples of compounds are provided in Table 1, which may be obtained from Maybridge (England):

TABLE 1

| Compound | | |
|---|---|---|
| Designation | Chemical Name | Structure |
| BTB 03160 | 4-(4-chlorophenyl)-6-methoxy-2-oxo- 1,2-dihydropyridine-3,5-dicarbonitrile | 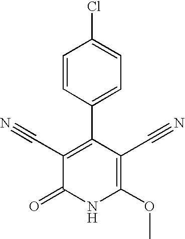 |
| BTB 03173 | 6-methoxy-2-oxo-4-(3,4,5- trimethoxyphenyl)-1,2- dihydropyridine-3,5-dicarbonitrile | 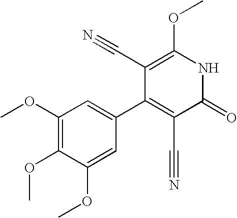 |
| BTB 09160 | 6-methyl-2-oxo-5-(2-phenyl-1,3- thiazol-4-yl)-1,2-dihydropyridine-3- carbonitrile | 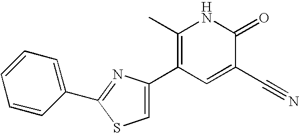 |
| BTB 09214 | 6-methyl-5-(2-methyl-1,3-thiazol-4- yl)-2-oxo-1,2-dihydropyridine-3- carbonitrile | 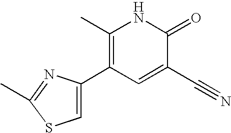 |
| BTB 09261 | 5-{2-[(3-fluorophenyl)thio]acetyl}-6- methyl-2-oxo-1,2-dihydropyridine-3- carbonitrile | 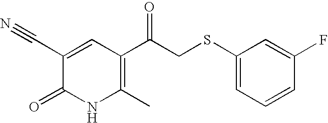 |
| BTB 14328 | diethyl 4-(chlorophenyl)-2,6- dimethyl-1,4-dihydropyridine-3,5- dicarboxylate | 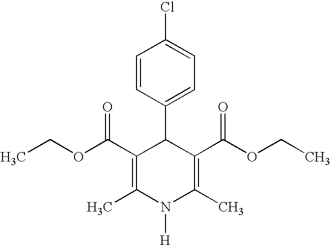 |
| BTB 14330 | diethyl 4-(4-hydroxy-3- methoxyphenyl)-2,6-dimethyl-1,4- dihydropyridine-3,5-dicarboxylate | 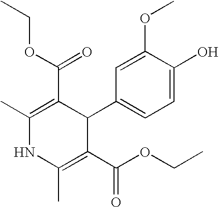 |
| BTB 14332 | diethyl 4-(2-furyl)-2,6-dimethyl-1,4- dihydropyridine-3,5-dicarboxylate | 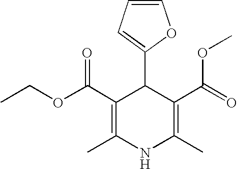 |
| CD 04170 | diethyl 4-{5-[3,5- di(trifluoromethyl)phenyl]-2-furyl}- 2,6-dimethyl-1,4-dihydropyridine-3,5- dicarboxylate | 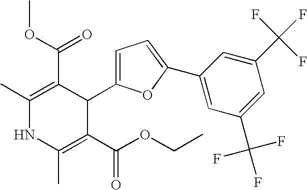 |
| HC 00063 | methyl 1-(2,5-dimethoxybenzyl)-5- fluoro-4-oxo-1,4-dihydropyridine-3- carboxylate | 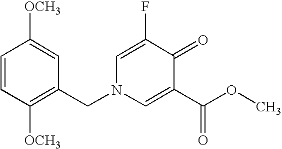 |
| HC 00065 | methyl 5-fluoro-4-oxo-1-[4- (trifluoromethyl)benzyl]-1,4- dihydropyridine-3-carboxylate | 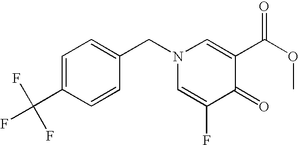 |
| HTS 00599 | 3,3-dimethyl-1-(4- morpholinophenyl)dihydropyridine- 2,6(1H,3H)-dione | 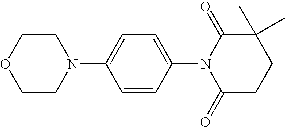 |
| HTS 01512 | 1-cyclohexyl-5-phenyl-1,6-dihydro- 2,3-pyridinedione | 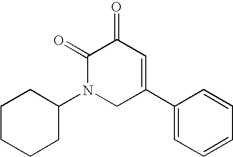 |
| HTS 07578 | 4-(1,3-diphenyl-1H-pyrazol-4-yl)-2- oxo-6-phenyl-1,2-dihydro-3- pyridinecarbonitrile | 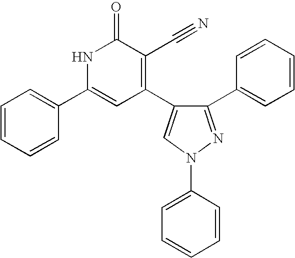 |
| HTS 09043 | 4-methyl-2-oxo-6-phenyl-1,2-dihydro- 3-pyridinecarbonitrile | 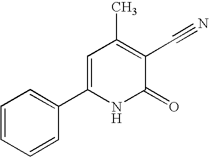 |
| HTS 10306 | 2-oxo-6-phenyl-4-(2-thienyl)-1,2- dihydro-3-pyridinecarbonitrile | 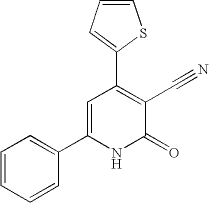 |
| HTS 10308 | 4,6-di(2-furyl)-2-oxo-1,2-dihydro-3- pyridinecarbonitrile | 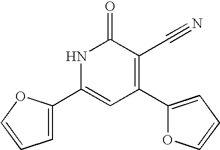 |
| HTS 10309 | 4-(2-furyl)-6-(4-methylphenyl)- 2-oxo-1,2-dihydro-3- pyridinecarbonitrile | 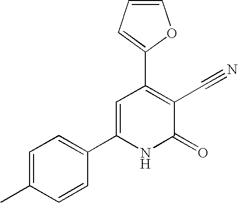 |
| HTS 10310 | 4-(2-furyl)-2-oxo-6-phenyl-1,2- dihydro-3-pyridinecarbonitrile | 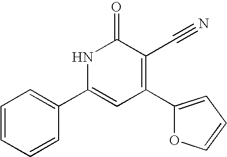 |
| JFD 01209 | (diethyl 4-(4-bromophenyl)-2,6- dimethyl-1,4-dihydropyridine-3,5- dicarboxylate) | 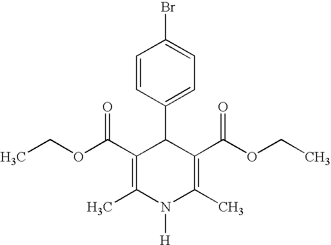 |
| JFD 03265 | 6-dimethyl-4-(4-nitrophenyl)-1,4- dihydropyridine-3,5-dicarbonitrile | 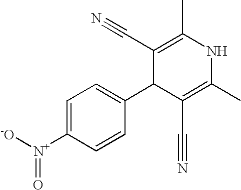 |
| JFD 03266 | (diethyl 2,6-dimethyl-4-(4- nitrophenyl)-1,4-dihydropyridine-3,5- dicarboxylate | 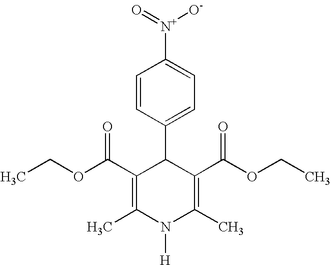 |
| JFD 03267 | 4-(2,4-dinitrophenyl)-2,6-dimethyl- 1,4-dihydropyridine-3,5-dicarbonitrile | 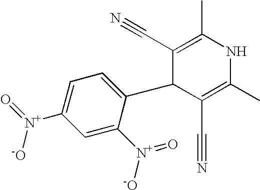 |
| JFD 03268 | 4-[4-(benzyloxy)phenyl]-2,6- dimethyl-1,4-dihydropyridine-3,5- dicarbonitrile | 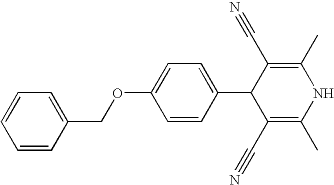 |
| JFD 03269 | dimethyl 4-(2,4-dinitrophenyl)-2,6- dimethyl-1,4-dihydropyridine-3,5- dicarboxylate | 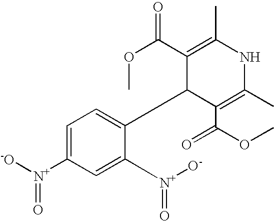 |
| JFD 03273 | 4-(3-chlorophenyl)-2,6-dimethyl-1,4- dihydropyridine-3,5-dicarbonitrile | 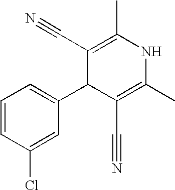 |
| JFD 03274 | diethyl 4-(3-chlorophenyl)-2,6- dimethyl-1,4-dihydropyridine-3,5- dicarboxylate | 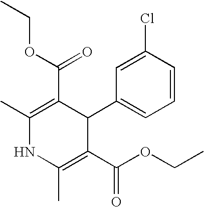 |
| JFD 03282 | (diethyl 2,6-dimethyl-4-(4- methylphenyl)-1,4-dihydropyridine- 3,5-dicarboxylate) | 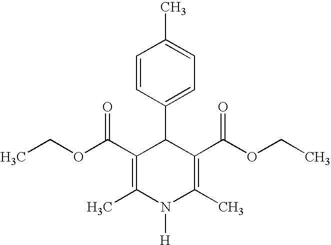 |
| JFD 03292 | 4-(3,4-dichlorophenyl)-2,6-dimethyl- 1,4-dihydropyridine-3,5-dicarbonitrile | 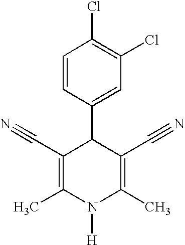 |
| JFD 03293 | dimethyl 4-(3,4-dichlorophenyl)-2,6- dimethyl-1,4-dihydropyridine-3,5- dicarboxylate | 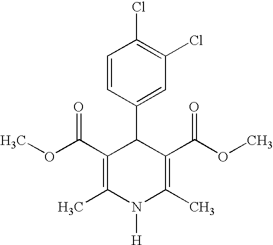 |
| JFD 03294 | (diethyl 4-(3,4-dichlorophenyl)-2,6- dimethyl-1,4-dihydropyridine-3,5- dicarboxylate) | 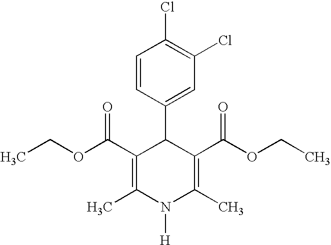 |
| JFD 03305 | (diethyl 4-(2-chlorophenyl)-2,6- dimethyl-1,4-dihydropyridine-3,5- dicarboxylate) | 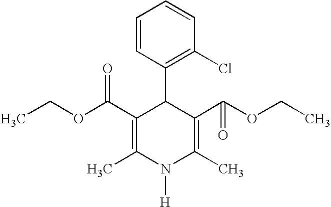 |
| JFD 03307 | dimethyl 2,6-dimethyl-4-(2- nitrophenyl)-1,4-dihydropyridine-3,5- dicarboxylate | 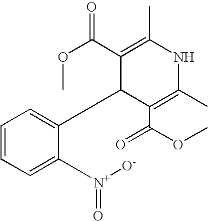 |
| JFD 03311 | diethyl 2,6-dimethyl-4-(2- nitrophenyl)-1,4-dihydropyridine-3,5- dicarboxylate | 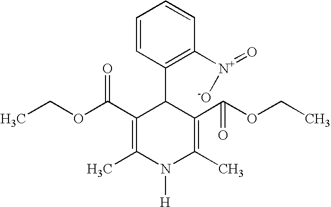 |
| JFD 03312 | 4-(3-methoxyphenyl)-2,6-dimethyl- 1,4-dihydropyridine-3,5-dicarbonitrile | 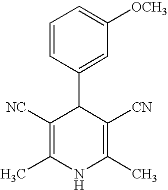 |
| JFD 03318 | diethyl 4-(4-fluorophenyl)-2,6- dimethyl-1,4-dihydropyridine-3,5- dicarboxylate | 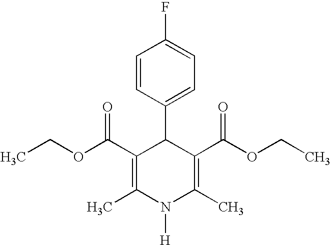 |
| PD 00088 | 1-acetyl-4,6-di(4-methylphenyl)-2- oxo-1,2-dihydropyridine-3- carbonitrile | 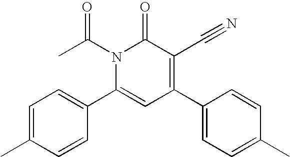 |
| PD 00090 | 6-(4-methylphenyl)-4-(3-nitrophenyl)- 2-oxo-1,2-dihydro-3- pyridinecarbonitrile | 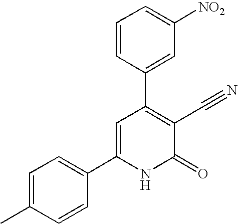 |
| PD 00463 | 1-[4-(4-chlorophenoxy)phenyl]-4- phenyldihydropyridine-2,6(1H,3H)- dione | 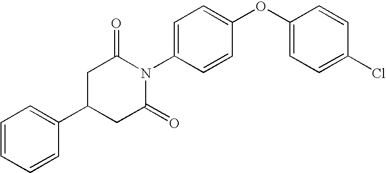 |
| PD 00700 | 2-(propylthio)-N-[4- (trifluoromethoxy)phenyl]-1,2- dihydropyridine-3-carboxamide | 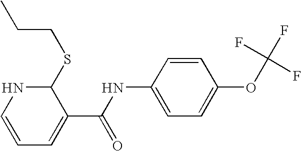 |
| RF 04555 | N˜1˜-(2,4-dichlorophenyl)-4- (trifluoromethyl)-5,6-dihydropyridine- 1,3(4H)-dicarboxamide | 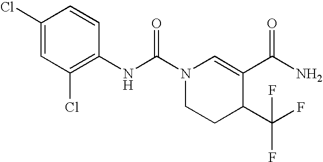 |
| RF 04777 | N-(4-chlorophenyl)-N,1-dimethyl-6- oxo-4-(trifluoromethyl)-1,6-dihydro- 3-pyridinecarboxamide | 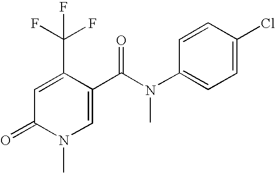 |
| RF 04780 | N-(4-chlorophenyl)-1-ethyl-N-methyl- 6-oxo-4-(trifluoromethyl)-1,6- dihydropyridine-3-carboxamide | 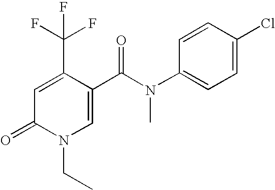 |
| RF 04781 | N-(3,4-dichlorophenyl)-N,1-dimethyl- 6-oxo-4-(trifluoromethyl)-1,6- dihydro-3-pyridinecarboxamide | 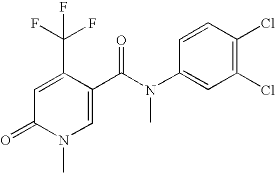 |
| RH 02165 | 2-oxo-6-pyridin-3-yl-4- (trifluoromethyl)-1,2-dihydropyridine- 3-carbonitrile | 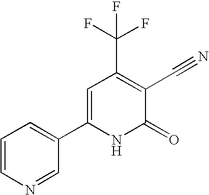 |
| RH 02186 | 1-amino-2-oxo-6-phenyl-4- (trifluoromethyl)-1,2-dihydropyridine- 3-carbonitrile | 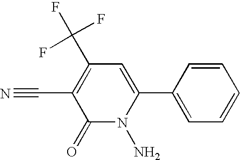 |
| RJC 03342 | 4-hydroxy-2-methyl-6-oxo-5-phenyl- 1,6-dihydropyridine-3-carbonitrile | 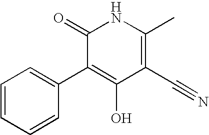 |
| RJC 03403 | diethyl 4-(2,4-dichlorophenyl)-2,6- dimethyl-1,4-dihydro-3,5- pyridinedicarboxylate | 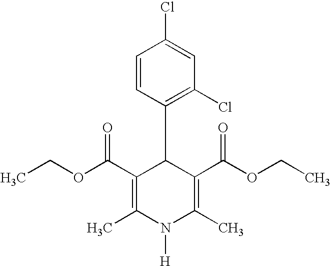 |
| RJC 03405 | diethyl 2,6-dimethyl-4-{5-[2- (trifluoromethyl)phenyl]-2-furyl}-1,4- dihydro-3,5-pyridinedicarboxylate | 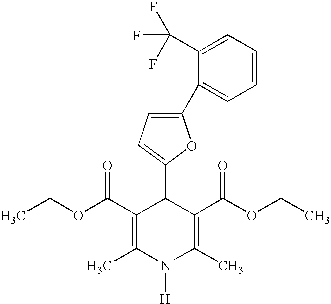 |
| RJC 03410 | diethyl 2,6-dimethyl-4-(6-methyl-2- pyridyl)-1,4-dihydro-3,5- pyridinedicarboxylate | 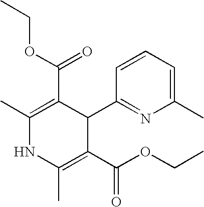 |
| RJC 03413 | diethyl 4-(2-chloro-4- methoxyphenyl)-2,6-dimethyl-1,4- dihydro-3,5-pyridinedicarboxylate | 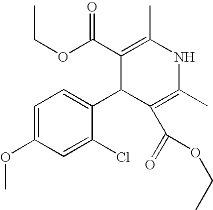 |
| RJC 03416 | dimethyl 2,6-dimethyl-4-{5-[2- (trifluoromethyl)phenyl]-2-furyl}-1,4- dihydro-3,5-pyridinedicarboxylate | 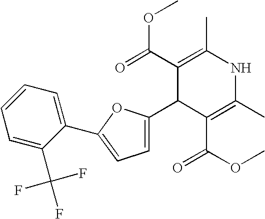 |
| RJC 03418 | dimethyl 4-(2-methoxyphenyl)-2,6- dimethyl-1,4-dihydro-3,5- pyridinedicarboxylate | 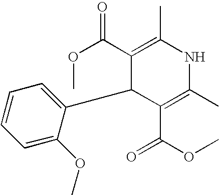 |
| RJC 03419 | 2,6-dimethyl-4-{5-[2- (trifluoromethyl)phenyl]-2-furyl}-1,4- dihydro-3,5-pyridinedicarbonitrile | 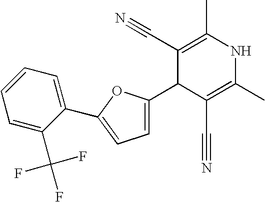 |
| RJC 03423 | dimethyl 4-(2,4-dichlorophenyl)-2,6- dimethyl-1,4-dihydro-3,5- pyridinedicarboxylate | 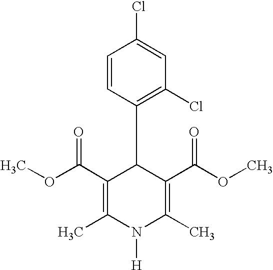 |
| RJC 03424 | 4-(2-chloro-4-hydroxyphenyl)-2,6- dimethyl-1,4-dihydro-3,5- pyridinedicarbonitrile | 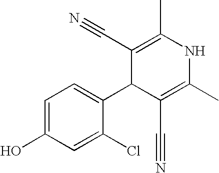 |
| RJC 03427 | 4-(3,4-dimethoxyphenyl)-2,6- dimethyl-1,4-dihydro-3,5- pyridinedicarbonitrile | 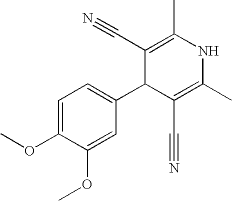 |
| RJC 03437 | dimethyl 2,6-dimethyl-4-(6-methyl-2- pyridyl)-1,4-dihydro-3,5- pyridinedicarboxylate | 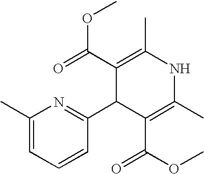 |
| S 14471 | 4-(4-chlorophenyl)-6-(4- isobutylphenyl)-2-oxo-1,2- dihydropyridine-3-carbonitrile | 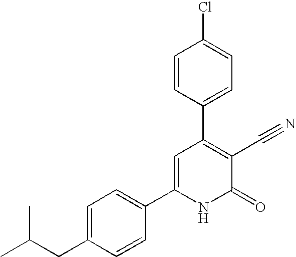 |
| SEW 02066 | dimethyl 2,6-dimethyl-4-(3-thienyl)- 1,4-dihydro-3,5-pyridinedicarboxylate | 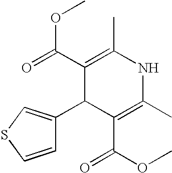 |
| SEW 02070 | dimethyl 4-{5-[2-(methoxycarbonyl)- 3-thienyl]-2-furyl}-2,6-dimethyl-1,4- dihydropyridine-3,5-dicarboxylate | 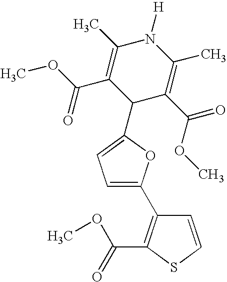 |
| XBX 00343 | diethyl 2,6-dimethyl-4-(3- nitrophenyl)-1,4-dihydropyridine-3,5- dicarboxylate | 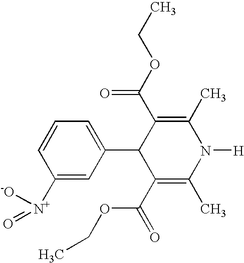 |

TABLE 2

| | | | |
|---|---|---|---|
| 2-11 | 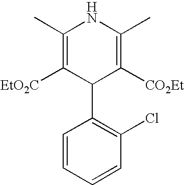 | 2-18 | 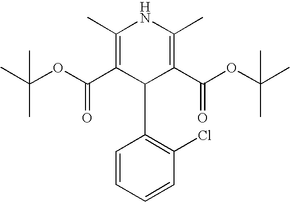 |
| 2-14 | 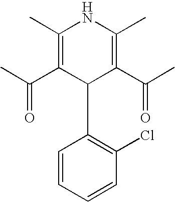 | 2-19 | 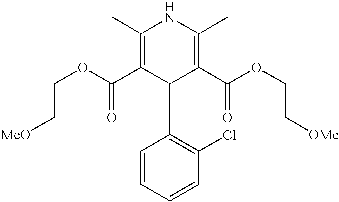 |
| 2-17 | 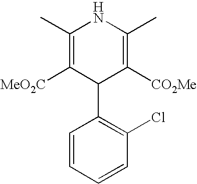 | 2-23 | 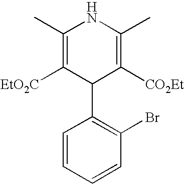 |
| 2-27 | 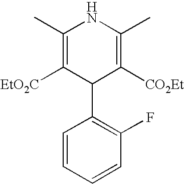 | 2-44 | 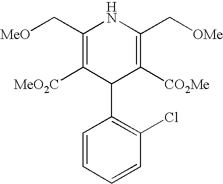 |
| 2-28 | 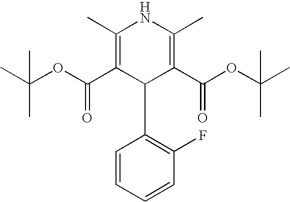 | 2-45 | 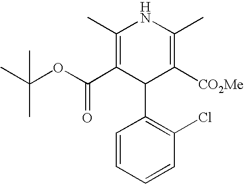 |
| 2-29 | 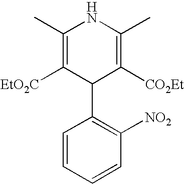 | 2-46 | 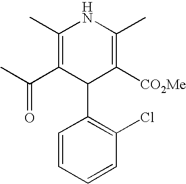 |
| 2-32 | 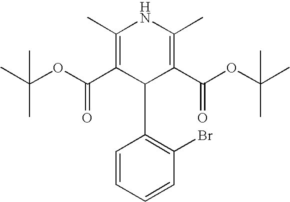 | 2-47 | 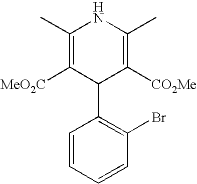 |
| 2-33 | 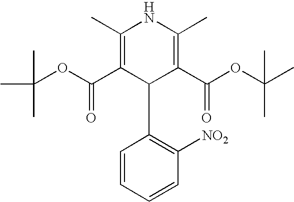 | 2-48 | 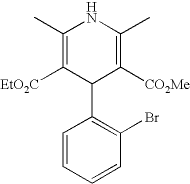 |
| 2-37 | 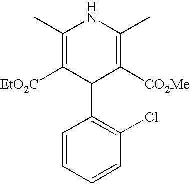 | 2-49 | 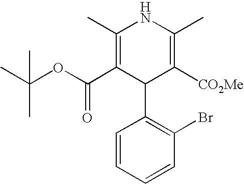 |
| 2-42 | 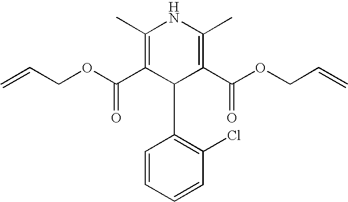 | 2-50 | 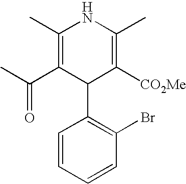 |
| 2-51 | 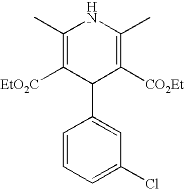 | 3-1 | 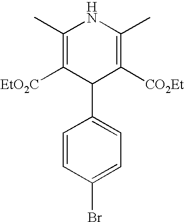 |
| 2-52 | 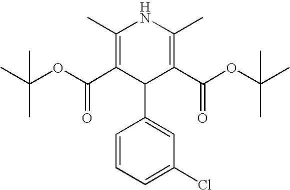 | 3-2 | 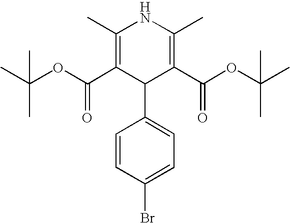 |
| 2-53 | 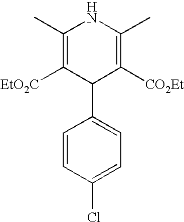 | 3-3 | 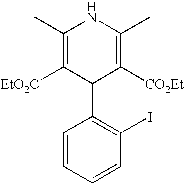 |
| 2-54 | 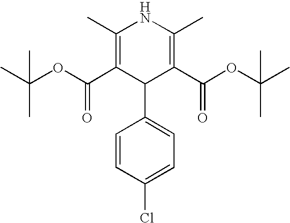 | 3-4 | 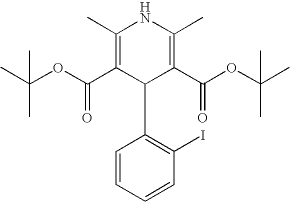 |
| 2-55 | 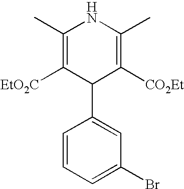 | 3-5 | 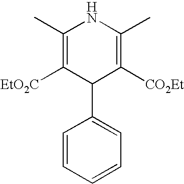 |
| 2-56 | 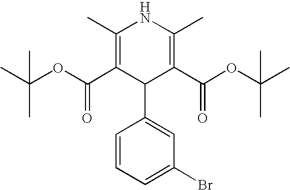 | 3-6 | 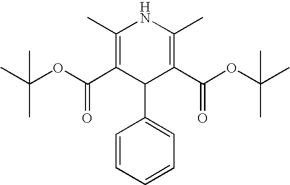 |
| | | 3-7 | 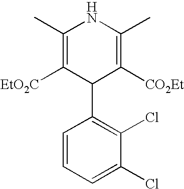 |
| 3-8 | 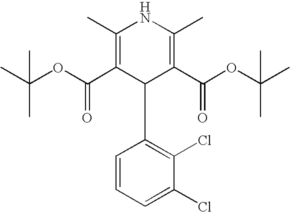 | 3-23 | 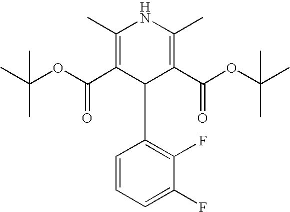 |
| 3-9 | 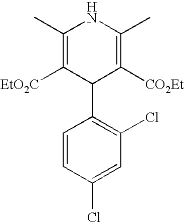 | 3-28 | 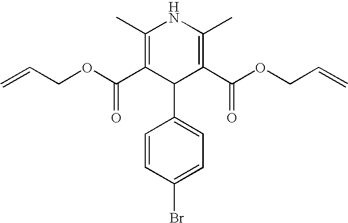 |
| 3-11 | 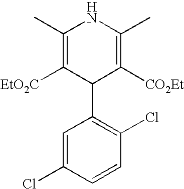 | 3-31 | 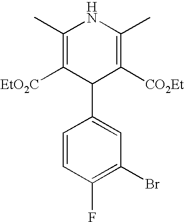 |
| 3-12 | 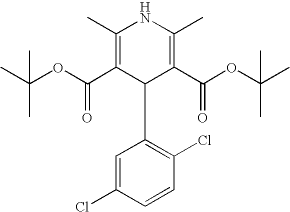 | 3-32 | 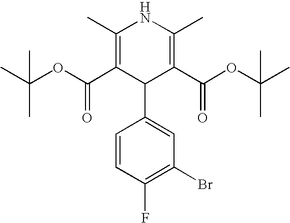 |
| 3-13 | 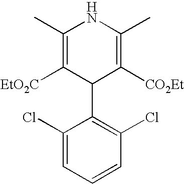 | 3-33 | 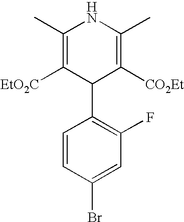 |
| 3-20 | 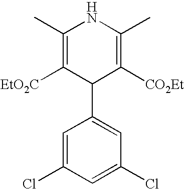 | 3-34 | 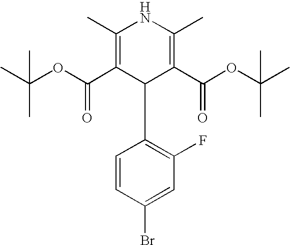 |
| 3-22 | 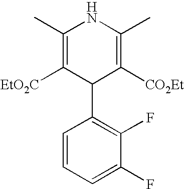 | | |
| 3-37 | 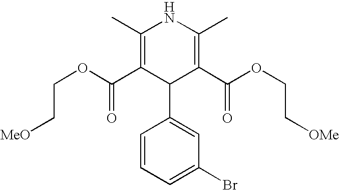 | 3-48 | 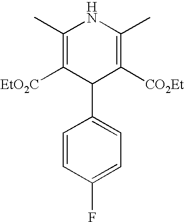 |
| 3-38 | 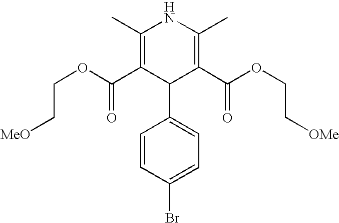 | 3-49 | 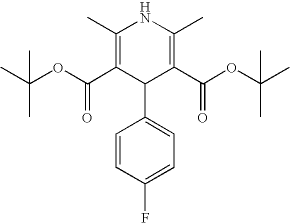 |
| 3-41 | 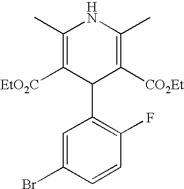 | 4-6 | 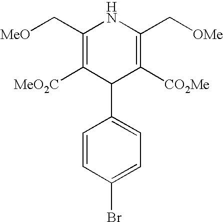 |
| 3-42 | 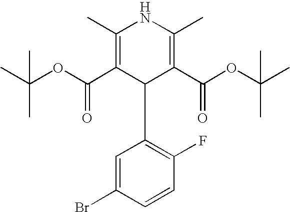 | 4-16 | 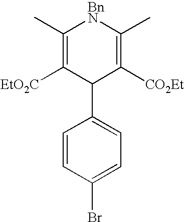 |
| 3-46 | 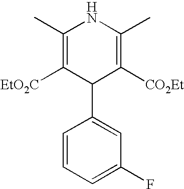 | 4-21 | 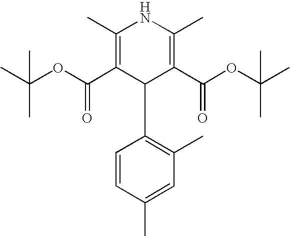 |
| 3-47 | 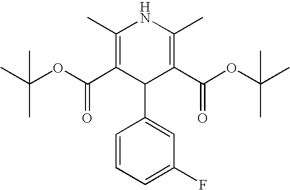 | | |

In another embodiment, the following compounds are provided, listed in Table 3, which can be used in the methods described herein:

TABLE 3

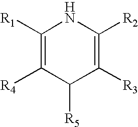

| R₁ | R₂ | R₃ | R₄ | R₅ |
|---|---|---|---|---|
| CH₃ | CH₃ | CO₂Et | CO₂Et | 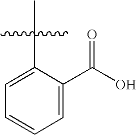 |
| CH₃ | CH₃ | CO₂Et | CO₂Et | 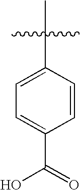 |
| CH₃ | CH₃ | CO₂Et | CO₂Et | 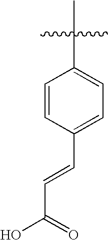 |
| CH₃ | CH₃ | CO₂Et | CO₂Et | 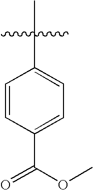 |
| CH₃ | CH₃ | CO₂Et | CO₂Et | 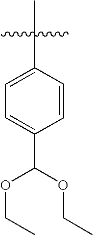 |
| CH₃ | CH₃ | CO₂Et | CO₂Et | 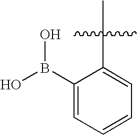 |
| CH₃ | CH₃ | CO₂Et | CO₂Et | 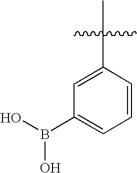 |
| CH₃ | CH₃ | CO₂Et | CO₂Et | 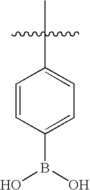 |
| CH₃ | CH₃ | CO₂Et | CO₂Et | 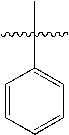 |
| CH₃ | CH₃ | CO₂^{t}Bu | CO₂^{t}Bu | 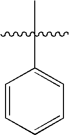 |
| CH₃ | CH₃ | CO₂Et | CO₂Et | 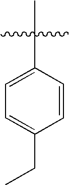 |
| CH₃ | CH₃ | CO₂Et | CO₂Et | 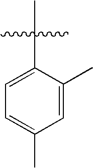 |
| CH₃ | CH₃ | CO₂Et | CO₂Et | 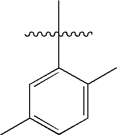 |
| CH₃ | CH₃ | CO₂Et | CO₂Et | 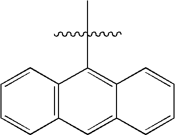 |
| CH₃ | CH₃ | CO₂Et | CO₂Et | 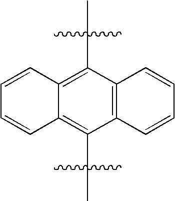 |
| CH₃ | CH₃ | CO₂Et | CO₂Et | 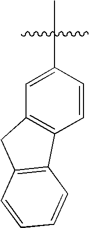 |
| CH₃ | CH₃ | CO₂Et | CO₂Et | 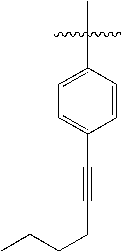 |
| CH₃ | CH₃ | CO₂Et | CO₂Et | 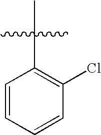 |
| CH₃ | CH₃ | C(O)CH₃ | C(O)CH₃ | 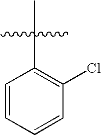 |
| CH₃ | CH₃ | CO₂Me | CO₂Me | 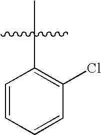 |
| CH₃ | CH₃ | CO₂^{t}Bu | CO₂^{t}Bu | 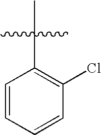 |
| CH₃ | CH₃ | CO₂(CH₂)OMe | CO₂(CH₂)OMe | 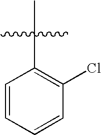 |
| CH₃ | CH₃ | CO₂Me | CO₂Et | 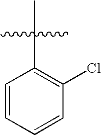 |
| CH₃ | CH₃ | CO₂CH₂CH═CH₂ | CO₂CH₂CH═CH₂ | 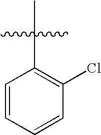 |
| CH₂OMe | CH₂OMe | CO₂Me | CO₂Me | 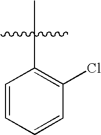 |
| CH₃ | CH₃ | CO₂Me | CO₂^{t}Bu | 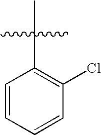 |
| CH₃ | CH₃ | CO₂Me | C(O)CH₃ | 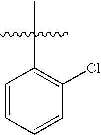 |
| CH₃ | CH₃ | CO₂Et | CO₂Et | 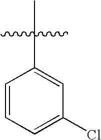 |
| CH₃ | CH₃ | CO₂^{t}Bu | CO₂^{t}Bu | 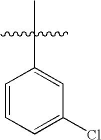 |
| CH₃ | CH₃ | CO₂Et | CO₂Et | 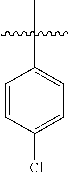 |
| CH₃ | CH₃ | CO₂^{t}Bu | CO₂^{t}Bu | 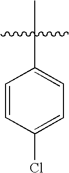 |
| CH₃ | CH₃ | CO₂Et | CO₂Et | 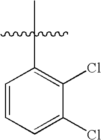 |
| CH₃ | CH₃ | CO₂^{t}Bu | CO₂^{t}Bu | 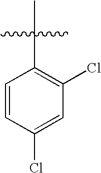 |
| CH₃ | CH₃ | CO₂Et | CO₂Et | 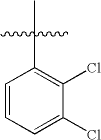 |
| CH₃ | CH₃ | CO₂Et | CO₂Et | 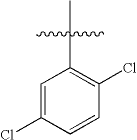 |
| CH₃ | CH₃ | CO₂^{t}Bu | CO₂^{t}Bu | 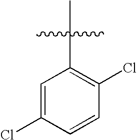 |
| CH₃ | CH₃ | CO₂Et | CO₂Et | 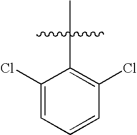 |
| CH₃ | CH₃ | CO₂Et | CO₂Et | 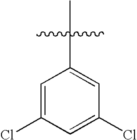 |
| CH₃ | CH₃ | CO₂Et | CO₂Et | 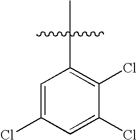 |
| CH₃ | CH₃ | CO₂Et | CO₂Et | 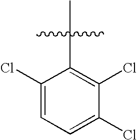 |
| CH₃ | CH₃ | CO₂Et | CO₂Et | 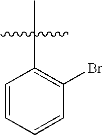 |
| CH₃ | CH₃ | CO₂^{t}Bu | CO₂^{t}Bu | 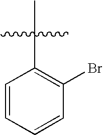 |
| CH₃ | CH₃ | CO₂Me | CO₂Me | 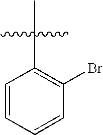 |
| CH₃ | CH₃ | CO₂Et | CO₂Et | 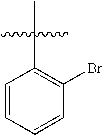 |
| CH₃ | CH₃ | CO₂Me | CO₂^{t}Bu | 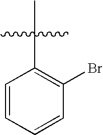 |
| CH₃ | CH₃ | CO₂Me | C(O)CH₃ | 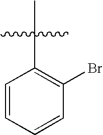 |
| CH₃ | CH₃ | CO₂Et | CO₂Et | 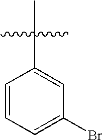 |
| CH₃ | CH₃ | CO₂^{t}Bu | CO₂^{t}Bu | 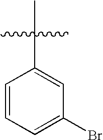 |
| CH₃ | CH₃ | CO₂Et | CO₂Et | 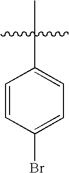 |
| CH₃ | CH₃ | CO₂^{t}Bu | CO₂^{t}Bu | 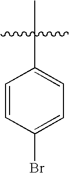 |
| CH₃ | CH₃ | CO₂Et | CO₂Et | 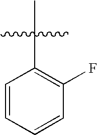 |
| CH₃ | CH₃ | CO₂^{t}Bu | CO₂^{t}Bu | 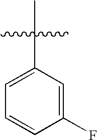 |
| CH₃ | CH₃ | CO₂Et | CO₂Et | 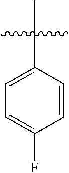 |
| CH₃ | CH₃ | CO₂Et | CO₂Et | 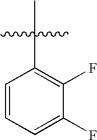 |
| CH₃ | CH₃ | CO₂Et | CO₂Et | 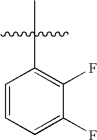 |
| CH₃ | CH₃ | CO₂^{t}Bu | CO₂^{t}Bu | 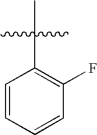 |
| CH₃ | CH₃ | CO₂Et | CO₂Et | 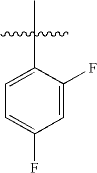 |
| CH₃ | CH₃ | CO₂Et | CO₂Et | 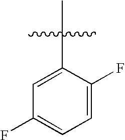 |
| CH₃ | CH₃ | CO₂Et | CO₂Et | 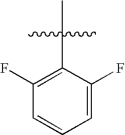 |
| CH₃ | CH₃ | CO₂Et | CO₂Et | 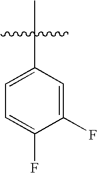 |
| CH₃ | CH₃ | CO₂Et | CO₂Et | 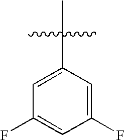 |
| CH₃ | CH₃ | CO₂Et | CO₂Et | 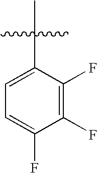 |
| CH₃ | CH₃ | CO₂Et | CO₂Et | 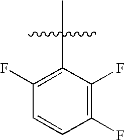 |
| CH₃ | CH₃ | CO₂Et | CO₂Et | 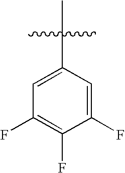 |
| CH₃ | CH₃ | CO₂Et | CO₂Et | 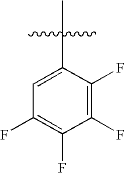 |
| CH₃ | CH₃ | CO₂Et | CO₂Et | 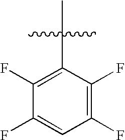 |
| CH₃ | CH₃ | CO₂Et | CO₂Et | 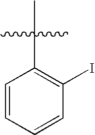 |
| CH₃ | CH₃ | CO₂^{t}Bu | CO₂^{t}Bu | 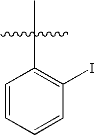 |
| CH₃ | CH₃ | CO₂Et | CO₂Et | 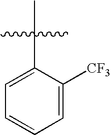 |
| CH₃ | CH₃ | CO₂Et | CO₂Et | 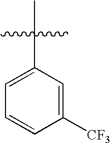 |
| CH₃ | CH₃ | CO₂Et | CO₂Et | 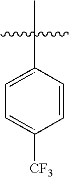 |
| CH₃ | CH₃ | CO₂Et | CO₂Et | 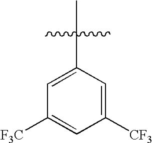 |
| CH₃ | CH₃ | CO₂Et | CO₂Et | 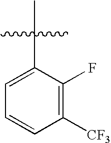 |
| CH₃ | CH₃ | CO₂Et | CO₂Et | 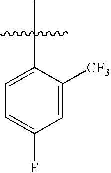 |
| CH₃ | CH₃ | CO₂Et | CO₂Et | 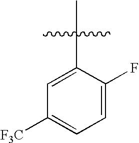 |
| CH₃ | CH₃ | CO₂Et | CO₂Et | 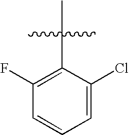 |
| CH₃ | CH₃ | CO₂Et | CO₂Et | 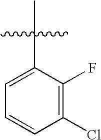 |
| CH₃ | CH₃ | CO₂Et | CO₂Et | 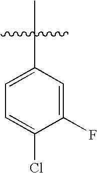 |
| CH₃ | CH₃ | CO₂Et | CO₂Et | 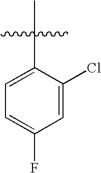 |
| CH₃ | CH₃ | CO₂Et | CO₂Et | 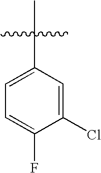 |
| CH₃ | CH₃ | CO₂Et | CO₂Et | 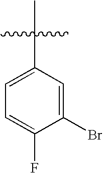 |
| CH₃ | CH₃ | CO₂Et | CO₂Et | 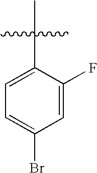 |
| CH₃ | CH₃ | CO₂Et | CO₂Et | 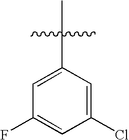 |
| CH₃ | CH₃ | CO₂Et | CO₂Et | 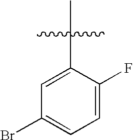 |
| CH₃ | CH₃ | CO₂Et | CO₂Et | 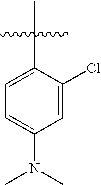 |
| CH₃ | CH₃ | CO₂Et | CO₂Et | 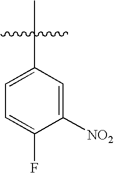 |
| CH₃ | CH₃ | CO₂Et | CO₂Et | 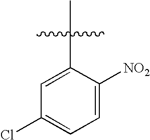 |
| CH₃ | CH₃ | CO₂Et | CO₂Et | 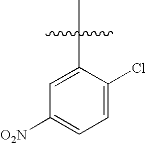 |
| CH₃ | CH₃ | CO₂Et | CO₂Et | 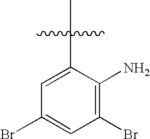 |
| CH₃ | CH₃ | CO₂Et | CO₂Et | 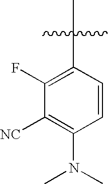 |
| CH₃ | CH₃ | CO₂Et | CO₂Et | 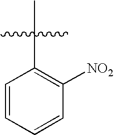 |
| CH₃ | CH₃ | CO₂^{t}Bu | CO₂^{t}Bu | 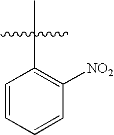 |
| CH₃ | CH₃ | CO₂Et | CO₂Et | 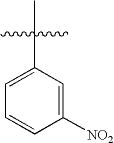 |
| CH₃ | CH₃ | CO₂Et | CO₂Et | 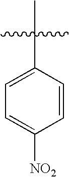 |
| CH₃ | CH₃ | CO₂Et | CO₂Et | 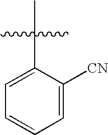 |
| CH₃ | CH₃ | CO₂Et | CO₂Et | 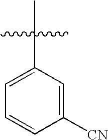 |
| CH₃ | CH₃ | CO₂Et | CO₂Et | 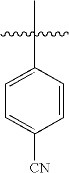 |
| CH₃ | CH₃ | CO₂Et | CO₂Et | 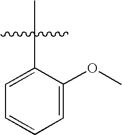 |
| CH₃ | CH₃ | CO₂Et | CO₂Et | 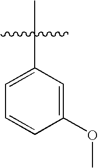 |
| CH₃ | CH₃ | CO₂Et | CO₂Et | 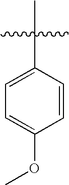 |
| CH₃ | CH₃ | CO₂Et | CO₂Et | 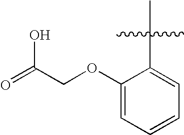 |
| CH₃ | CH₃ | CO₂Et | CO₂Et | 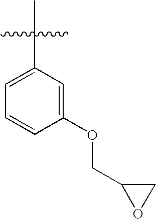 |
| CH₃ | CH₃ | CO₂Et | CO₂Et | 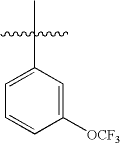 |
| CH₃ | CH₃ | CO₂Et | CO₂Et | 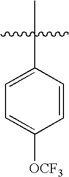 |
| CH₃ | CH₃ | CO₂Et | CO₂Et | 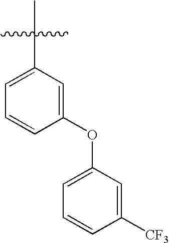 |
| CH₃ | CH₃ | CO₂Et | CO₂Et | 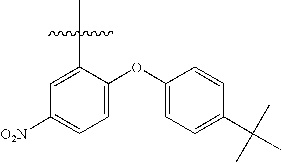 |
| CH₃ | CH₃ | CO₂Et | CO₂Et | 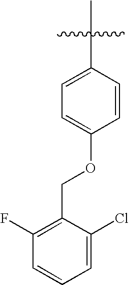 |
| CH₃ | CH₃ | CO₂Et | CO₂Et | 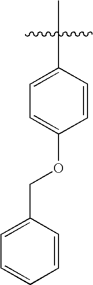 |
| CH₃ | CH₃ | CO₂Et | CO₂Et | 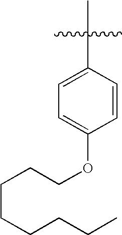 |
| CH₃ | CH₃ | CO₂Et | CO₂Et | 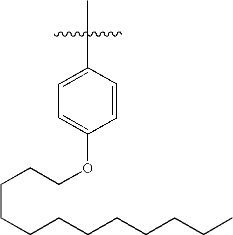 |
| CH₃ | CH₃ | CO₂Et | CO₂Et | 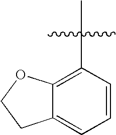 |
| CH₃ | CH₃ | CO₂Et | CO₂Et | 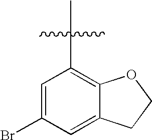 |
| CH₃ | CH₃ | CO₂Et | CO₂Et | 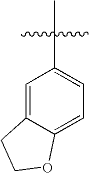 |
| CH₃ | CH₃ | CO₂Et | CO₂Et | 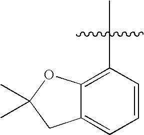 |
| CH₃ | CH₃ | CO₂Et | CO₂Et | 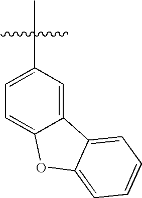 |
| CH₃ | CH₃ | CO₂Et | CO₂Et | 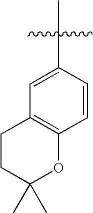 |
| CH₃ | CH₃ | CO₂Et | CO₂Et | 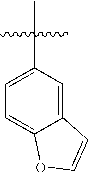 |
| CH₃ | CH₃ | CO₂Et | CO₂Et | 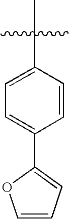 |
| CH₃ | CH₃ | CO₂Et | CO₂Et | 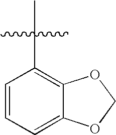 |
| CH₃ | CH₃ | CO₂Et | CO₂Et | 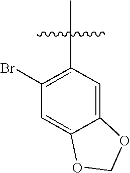 |
| CH₃ | CH₃ | CO₂Et | CO₂Et | 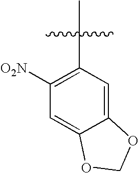 |
| CH₃ | CH₃ | CO₂Et | CO₂Et | 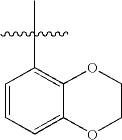 |
| CH₃ | CH₃ | CO₂Et | CO₂Et | 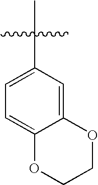 |
| CH₃ | CH₃ | CO₂Et | CO₂Et | 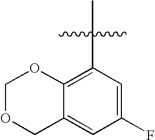 |
| CH₃ | CH₃ | CO₂Et | CO₂Et | 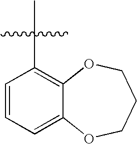 |
| CH₃ | CH₃ | CO₂Et | CO₂Et | 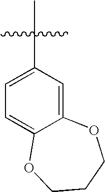 |
| CH₃ | CH₃ | CO₂Et | CO₂Et | 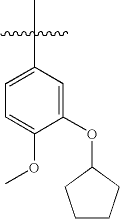 |
| CH₃ | CH₃ | CO₂Et | CO₂Et | 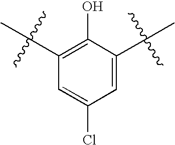 |
| CH₃ | CH₃ | CO₂Et | CO₂Et | 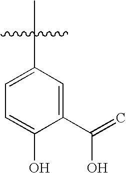 |
| CH₃ | CH₃ | CO₂Et | CO₂Et | 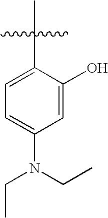 |
| CH₃ | CH₃ | CO₂Et | CO₂Et | 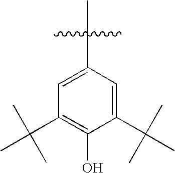 |
| CH₃ | CH₃ | CO₂Et | CO₂Et | 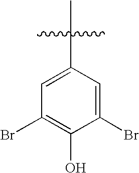 |
| CH₃ | CH₃ | CO₂Et | CO₂Et | 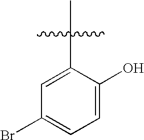 |
| CH₃ | CH₃ | CO₂Et | CO₂Et | 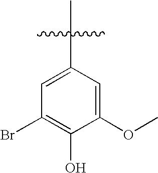 |
| CH₃ | CH₃ | CO₂Et | CO₂Et | 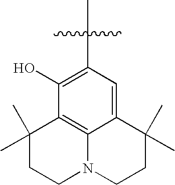 |
| CH₃ | CH₃ | CO₂Et | CO₂Et | 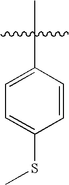 |
| CH₃ | CH₃ | CO₂Et | CO₂Et | 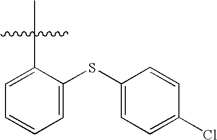 |
| CH₃ | CH₃ | CO₂Et | CO₂Et | 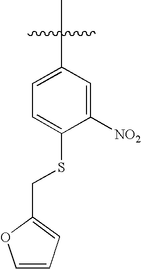 |
| CH₃ | CH₃ | CO₂Et | CO₂Et | 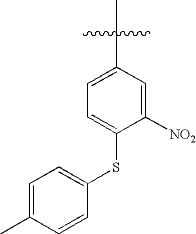 |
| CH₃ | CH₃ | CO₂Et | CO₂Et | 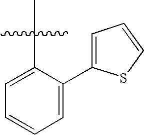 |
| CH₃ | CH₃ | CO₂Et | CO₂Et | 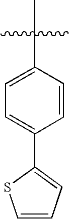 |
| CH₃ | CH₃ | CO₂Et | CO₂Et | 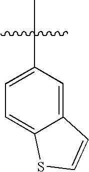 |
| CH₃ | CH₃ | CO₂Et | CO₂Et | 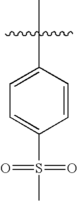 |
| CH₃ | CH₃ | CO₂Et | CO₂Et | 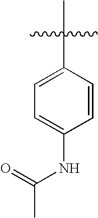 |
| CH₃ | CH₃ | CO₂Et | CO₂Et | 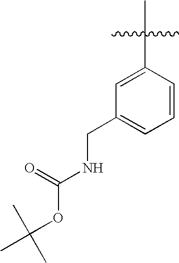 |
| CH₃ | CH₃ | CO₂Et | CO₂Et | 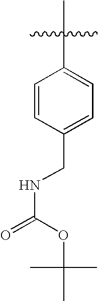 |
| CH₃ | CH₃ | CO₂Et | CO₂Et | 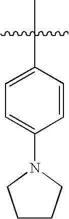 |
| CH₃ | CH₃ | CO₂Et | CO₂Et | 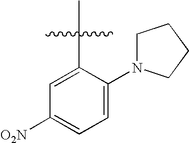 |
| CH₃ | CH₃ | CO₂Et | CO₂Et | 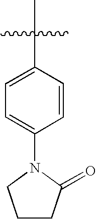 |
| CH₃ | CH₃ | CO₂Et | CO₂Et | 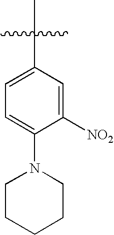 |
| CH₃ | CH₃ | CO₂Et | CO₂Et | 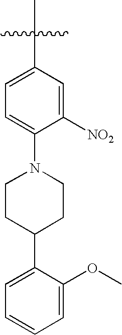 |
| CH₃ | CH₃ | CO₂Et | CO₂Et | 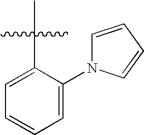 |
| CH₃ | CH₃ | CO₂Et | CO₂Et | 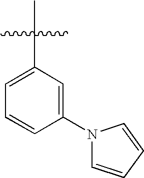 |
| CH₃ | CH₃ | CO₂Et | CO₂Et | 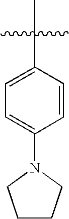 |
| CH₃ | CH₃ | CO₂Et | CO₂Et | 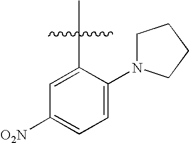 |
| CH₃ | CH₃ | CO₂Et | CO₂Et | 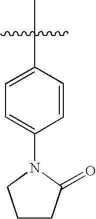 |
| CH₃ | CH₃ | CO₂Et | CO₂Et | 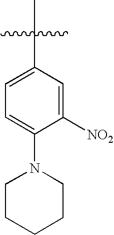 |
| CH₃ | CH₃ | CO₂Et | CO₂Et | 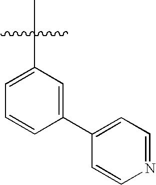 |
| CH₃ | CH₃ | CO₂Et | CO₂Et | 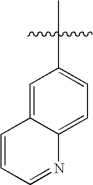 |
| CH₃ | CH₃ | CO₂Et | CO₂Et | 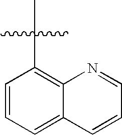 |
| CH₃ | CH₃ | CO₂Et | CO₂Et | 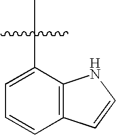 |
| CH₃ | CH₃ | CO₂Et | CO₂Et | 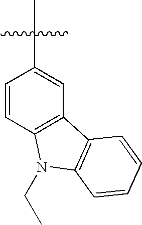 |
| CH₃ | CH₃ | CO₂Et | CO₂Et | 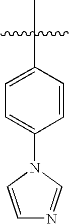 |
| CH₃ | CH₃ | CO₂Et | CO₂Et | 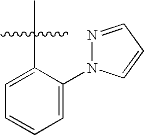 |
| CH₃ | CH₃ | CO₂Et | CO₂Et | 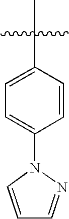 |
| CH₃ | CH₃ | CO₂Et | CO₂Et | 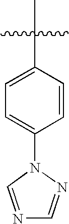 |
| CH₃ | CH₃ | CO₂Et | CO₂Et | 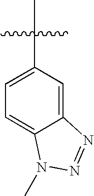 |
| CH₃ | CH₃ | CO₂Et | CO₂Et | 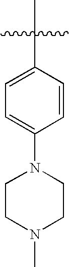 |
| CH₃ | CH₃ | CO₂Et | CO₂Et | 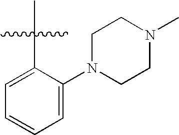 |
| CH₃ | CH₃ | CO₂Et | CO₂Et | 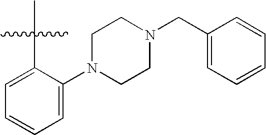 |
| CH₃ | CH₃ | CO₂Et | CO₂Et | 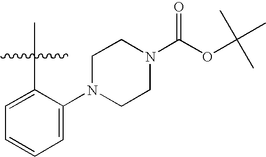 |
| CH₃ | CH₃ | CO₂Et | CO₂Et | 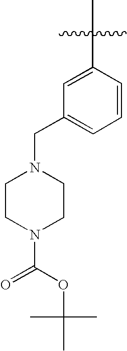 |
| CH₃ | CH₃ | CO₂Et | CO₂Et | 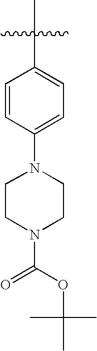 |
| CH₃ | CH₃ | CO₂Et | CO₂Et | 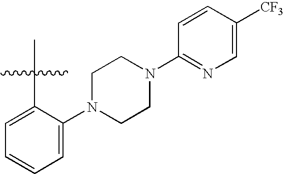 |
| CH₃ | CH₃ | CO₂Et | CO₂Et | 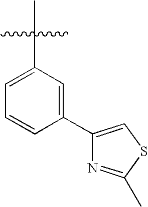 |
| CH₃ | CH₃ | CO₂Et | CO₂Et | 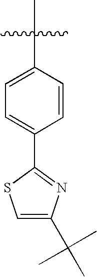 |
| CH₃ | CH₃ | CO₂Et | CO₂Et | 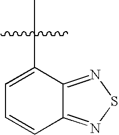 |
| CH₃ | CH₃ | CO₂Et | CO₂Et | 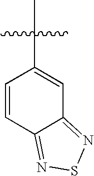 |
| CH₃ | CH₃ | CO₂Et | CO₂Et | 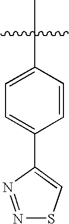 |
| CH₃ | CH₃ | CO₂Et | CO₂Et | 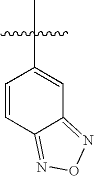 |
| CH₃ | CH₃ | CO₂Et | CO₂Et | 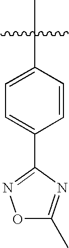 |
| CH₃ | CH₃ | CO₂Et | CO₂Et | 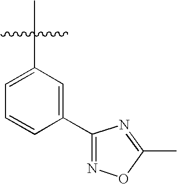 |
| CH₃ | CH₃ | CO₂Et | CO₂Et | 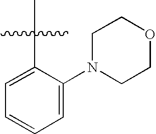 |
| CH₃ | CH₃ | CO₂Et | CO₂Et | 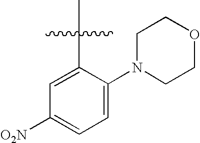 |
| CH₃ | CH₃ | CO₂Et | CO₂Et | 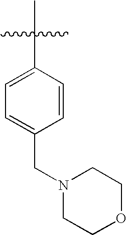 |
| CH₃ | CH₃ | CO₂Et | CO₂Et | 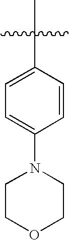 |
| CH₃ | CH₃ | CO₂Et | CO₂Et | 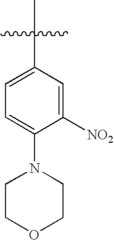 |
| CH₃ | CH₃ | CO₂Et | CO₂Et | 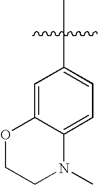 |

In another embodiment, the following compounds can be used in the methods described herein:

(S)-(+)-niguldipine ((S)-1,4-dihydro-2,6-dimethyl-4-(3-nitrophenyl)-3,5-pyridinedicarboxylic acid, 3-(4,4,-diphenyl-1-piperidinyl)propyl methyl ester hydrochloride), a dihydropyridine L-type Ca²⁺ channel blocker and α_{1A}-adrenoceptor antagonist, which is more active than the (R) enantiomer:

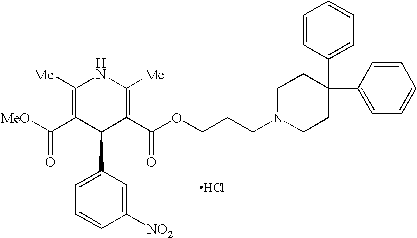

R-niguldipine ((R)-1,4-dihydro-2,6-dimethyl-4-(3-nitrophenyl)-3,5-pyridinedicarboxylic acid, 3-(4,4,-diphenyl-1-piperidinyl)propyl methyl ester hydrochloride), a digydropyridine L-type Ca²⁺ channel blocker and α_{1A}-adrenoceptor antagonist, which is less active than the (S) enantiomer:

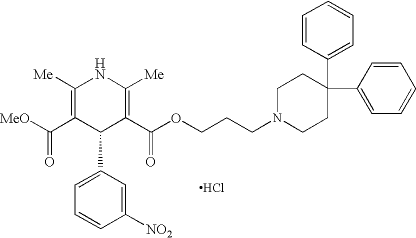

Furthermore, various NF-kB activation inhibitor compounds can be administered according to the treatment and diagnostic methods of the present invention, and include, without limitation the following compounds as well as prodrugs, derivatives and salts thereof Preferred are those compounds that decrease CCE, for example, by at least about 5%, 10%, 15%, 20% or more in cells.

Exemplary compounds:

artemisinin, an antimalarial agent extracted from the dry leaves of the Chinese herb Artemsisia annua (qinghaosu or sweet wormwood):

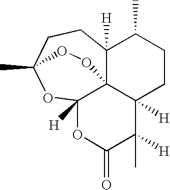

celastrol (3-hydroxy-24-nor-2-oxo-1(10),3,5,7,-friedelatetraen-29-oic acid (tripterin), a cell-permeable dienone-phenolic triterpene compound isolated from the Chinese Thunder of God vine (T. wilfordii) that exhibits antioxidant and anti-inflammatory properties:

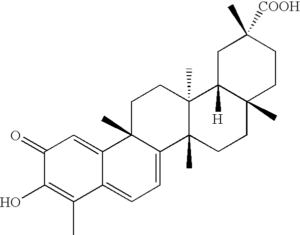

NF-kb Activation Inhibitor (6-amino-4-(4-phenoxyphenylethylamino)quinazoline) (a quinazoline), a cell-permeable quinazoline compound that acts as a potent inhibitor of NF-kB transcriptional activation and LPS-induced TNF-α production:

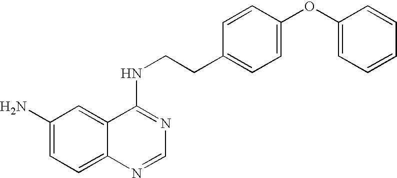

isoalantolactone, also referred to as isohelenin, a cell-permeable sesquiterpene lactone with anti-inflammatory properties that acts as a highly specific, potent, irreversible inhibitor of NF-kB activation by preventing I-kBa degradation:

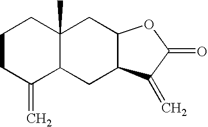

kamebakaurin, a cell-permeable kaurane diterpene analog containing a methylene-lactone functionality that displays anti-inflammatory properties and acts as a potent, irreversible inhibitor of NF-kB activation:

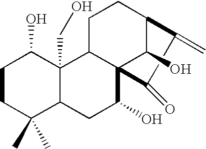

IKK-2 Inhibitor IV (5-(p-fluorophenyl)-2-ureido]thiophene-3-carboxamide), a cell-permeable (thienothienyl)amino-acetamide compound that displays anti-inflammatory properties, acts as a potent, reversible, ATP-competitive, and highly selective inhibitor of IKK-2, and has been shown to specifically block NF-kB-dependent gene expression in stimulated synovial fibroblasts:

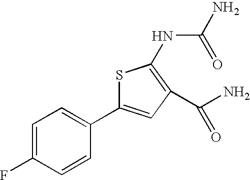

Other NF-kb Inhibitors useful in the methods and compositions disclosed herein include:

Capsaicin: 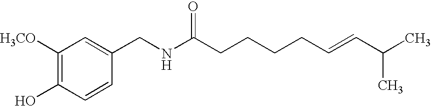
NF-kB SN50:

H-Ala-Ala-Val-Ala-Leu-Leu-Pro-Ala-Val-Leu-Leu-Ala-Leu-Leu-Ala-Pro-Val-Gln-Arg-Lys-Arg-Gln-Lys-Leu-Met-Pro-OH

Parthenolide, Tanacetum parthenium:

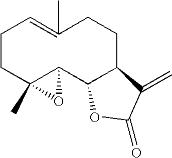

Andrographolide:

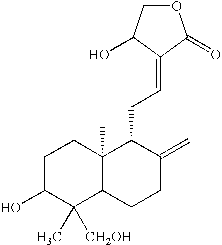

Caffeic Acid Phenethyl Ester (CAPE): 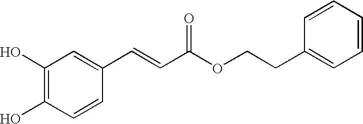
and hypoestoxide:

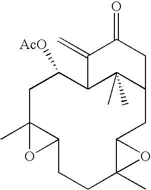

Other useful compounds include:

Fluphenazine-N-2chloroethane, Dihydrochloride (calmodulin antagonist):

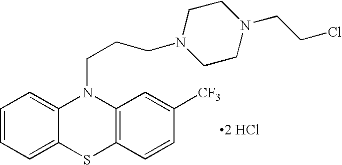

In another embodiment, the compound is one of the following compounds:

1,2-Bis(2-aminophenoxy)ethane N,N,N′,N′-tetraacetic acid acetoxymethyl ester (RN: 139890-68-9); also referred to as “Bapta-AM”; or: N-(2-((Acetyloxy)methoxy)-2-oxoethyl)-N-(2-(2-(2-(bis(carboxymethyl)amino)phenoxy)ethoxy)phenyl)glycine:

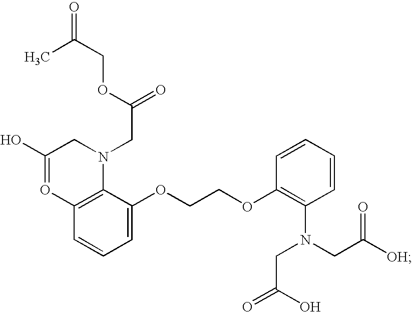

diltiazem:

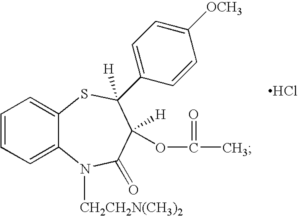

Isradipine:

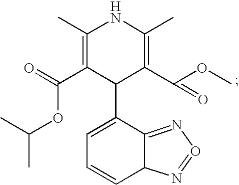

felodipine:

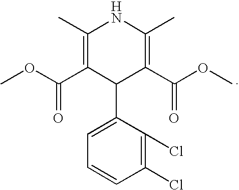

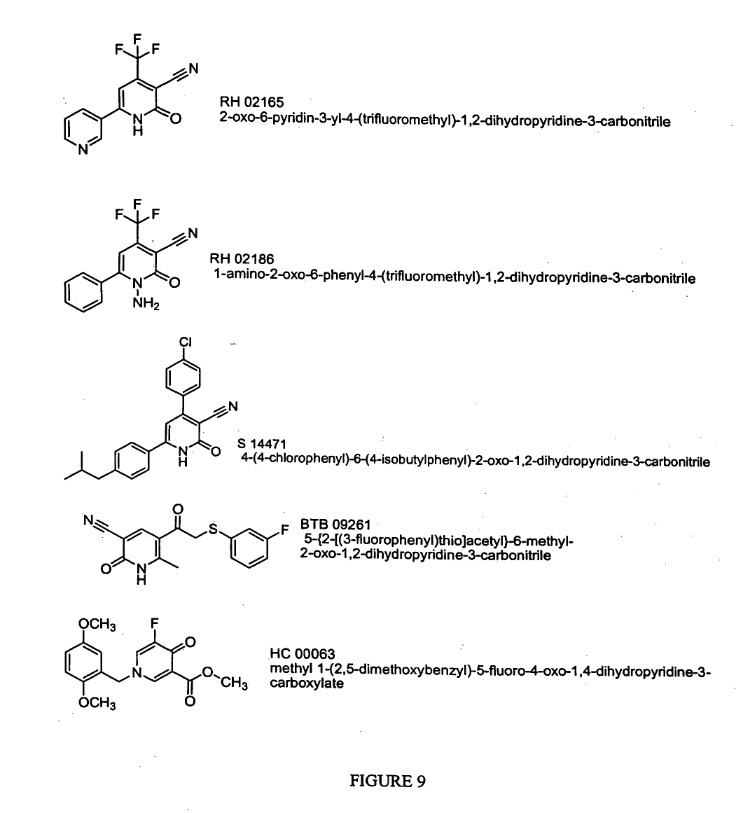

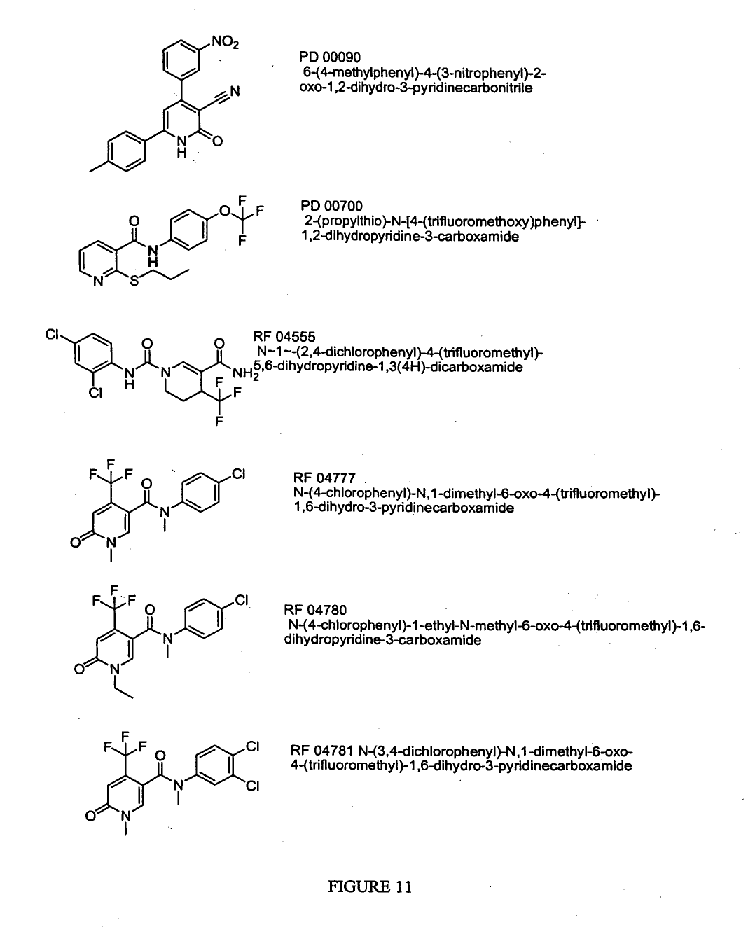

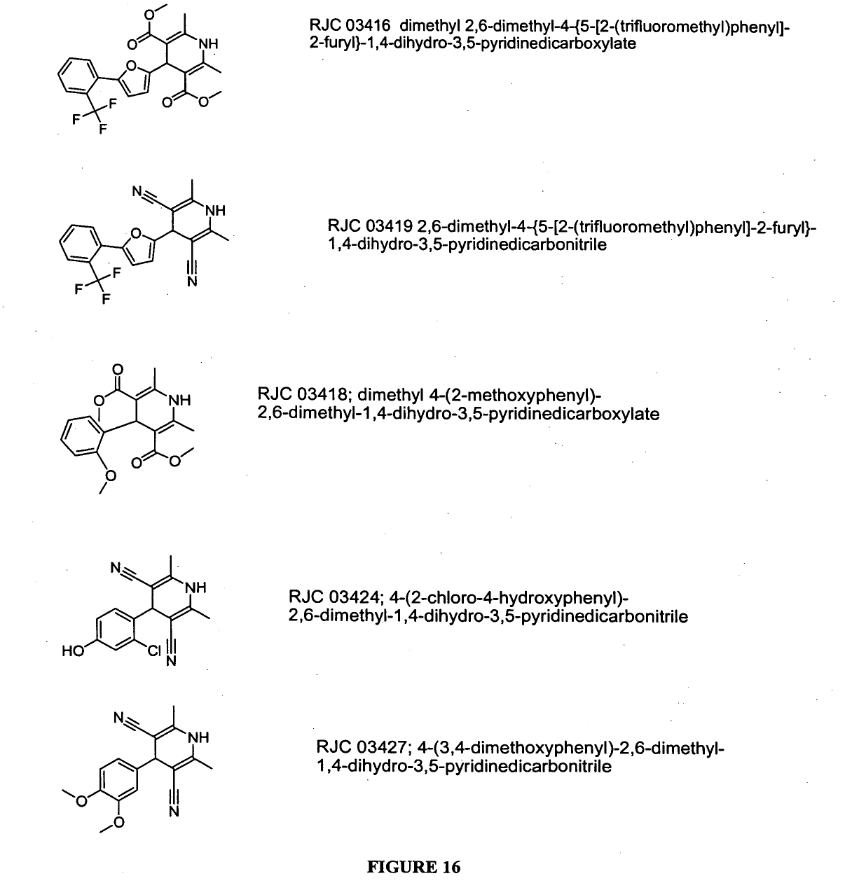

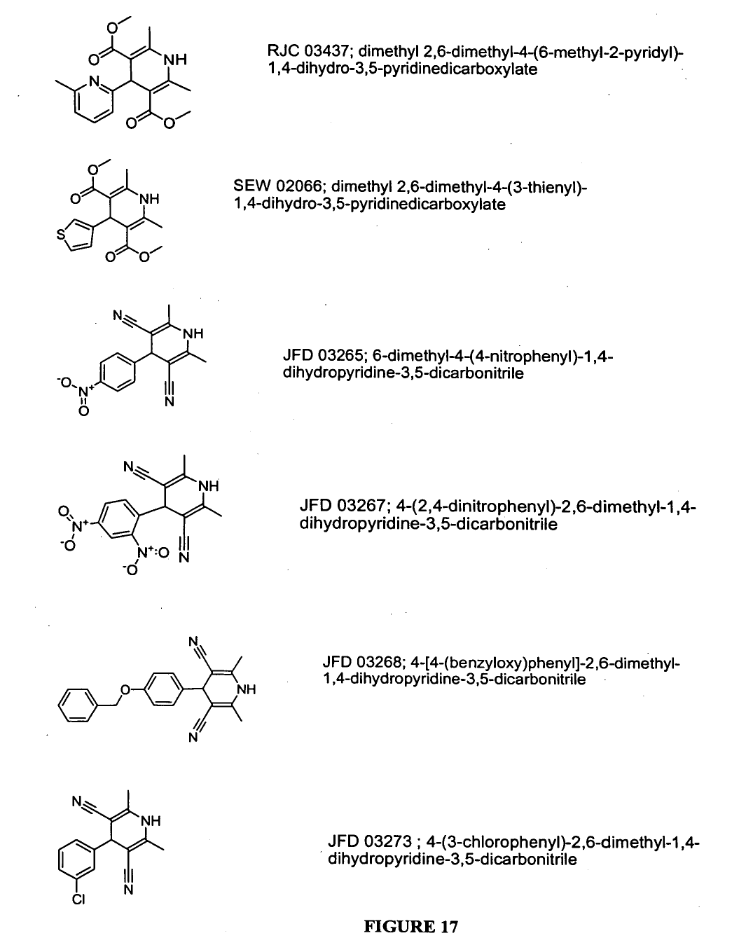

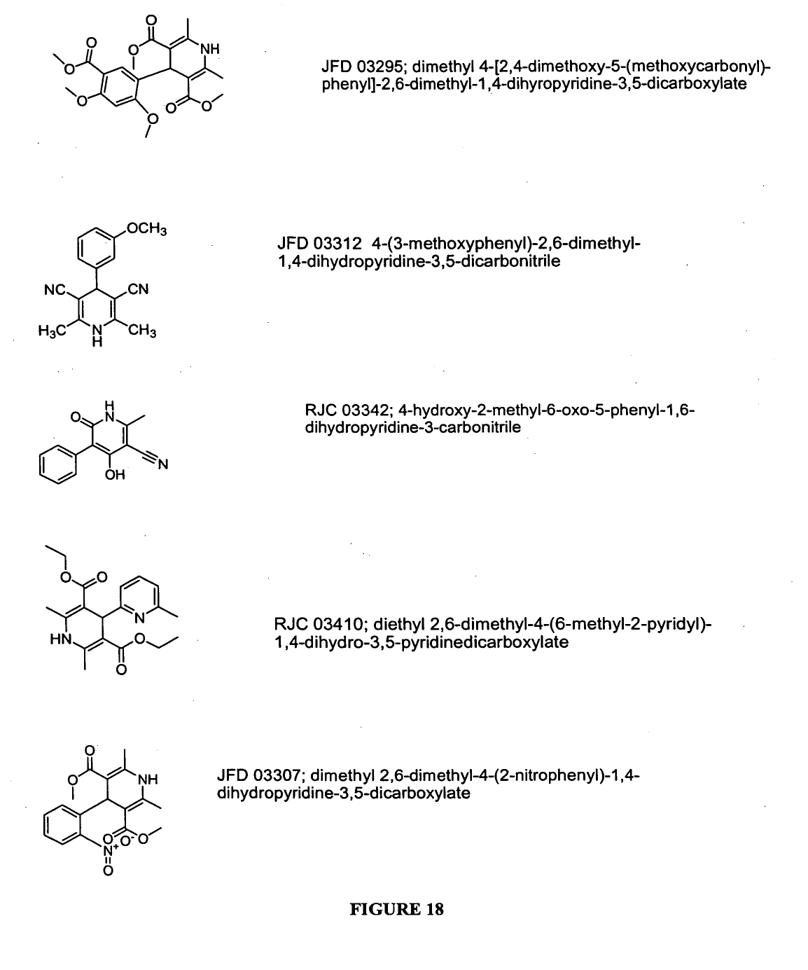

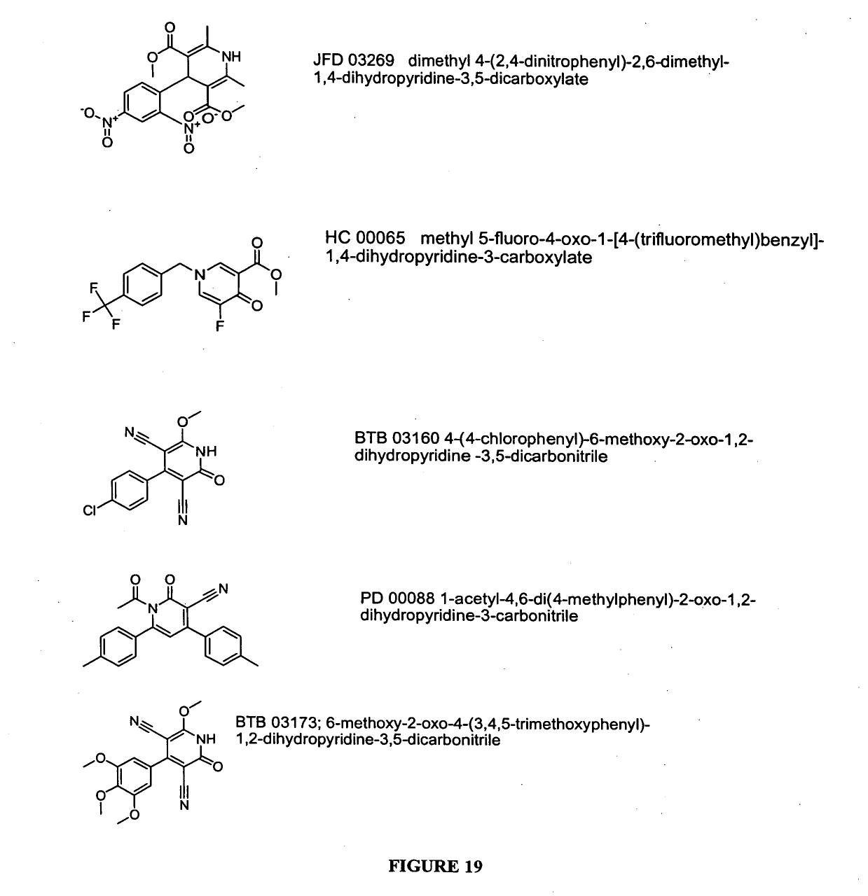

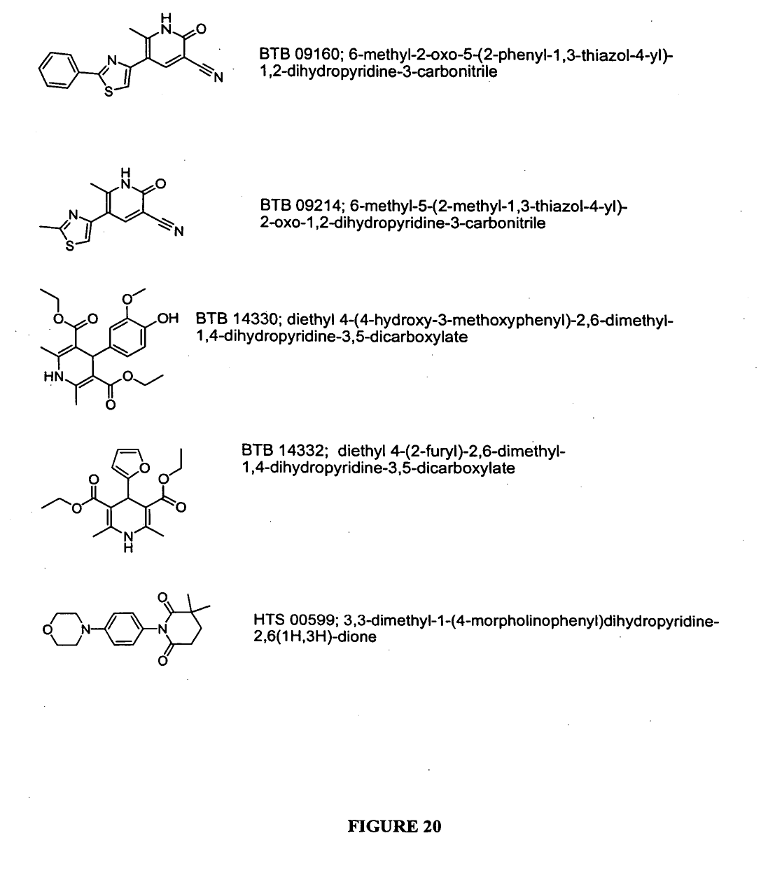

Exemplary compounds also are shown in FIGS. 9, 10 and 11. Further embodiments of compounds useful in the methods and compositions disclosed herein are shown in FIGS. 16-21. In one embodiment, the compound can decrease CCE, for example, by at least about 10% or more in cells that, e.g, overexpress APP or a fragment thereof, and optionally reduce β amyloid production, for example, by at least about 20% or more, in cultured cells which overexpress APP or a fragment thereof.

It is to be understood that the compounds disclosed herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is understood that the disclosure of a compound herein encompasses any racemic, optically active, polymorphic, or steroisomeric form, or mixtures thereof, which preferably possesses the useful properties described herein, it being well known in the art how to prepare optically active forms and how to determine activity using the standard tests described herein, or using other similar tests which are will known in the art. Examples of methods that can be used to obtain optical isomers of the compounds include the following:

i) physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or complete separation of a racemate by virtue of differing rates of reaction for the enantiomers with an enzyme

iv) enzymatic asymmetric synthesis, a synthetic technique whereby at least one step of the synthesis uses an enzymatic reaction to obtain an enantiomerically pure or enriched synthetic precursor of the desired enantiomer;

v) chemical asymmetric synthesis—a synthetic technique whereby the desired enantiomer is synthesized from an achiral precursor under conditions that produce asymetry (i.e., chirality) in the product, which may be achieved using chiral catalysts or chiral auxiliaries;

vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer,

viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography, a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;

xi) chiral gas chromatography, a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent; and

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

Synthesis of Compounds

Compounds useful in the methods and compositions described herein are in one embodiment available from commercially sources such as Maybridge, Cornwall, England, or EMD Calbiochem, San Diego, Calif.

In one embodiment, 3,5 disubstituted symmetrical dihydropyridine compounds are prepared by the reaction of two equivalents of alkylacetoacetate or other β-ketoester or β-ketoketone and one equivalent of an arylaldehyde dissolved in ethanol (˜4 equivalents) and NH₄OH (˜3 equivalents) at ambient temperature. The arylaldehyde compound used in the synthesis can be optionally substituted as desired. The alkyl group of the alkylacetoacetate reagent can be saturated or unsaturated or substituted as desired, to include substituents such as alkoxy. This mixture is, for example, stirred for 1 hour at ambient temperature followed by 2-3 hours at 80-100° C. The reaction mixture may then be cooled to ambient temperature, azeotroped with a solvent, such as toluene, and the product may be crystallized from a solvent, such as hot hexane, or a combination of solvents, such as ethyl acetate and hexane. In the reaction below, R is a desired group such as alkyl or substituted alkyl; R⁶ is a desired group such as optionally substituted alkyl, aryl, alkoxide, or aryloxide; and R¹, R², R³, R⁴, R⁵ are independently H, alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle.

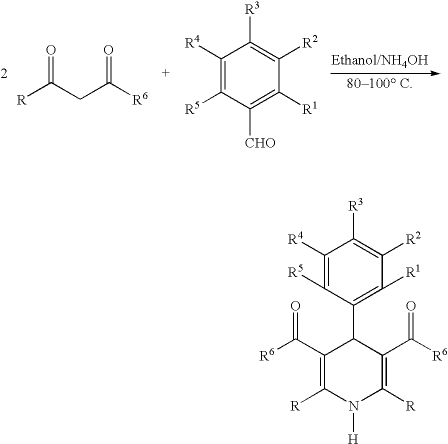

In another embodiment, 3,5 disubstituted unsymmetrical dihydropyridine compounds are prepared by reaction of one equivalent of alkylacetoacetate or other β-ketoester or β-ketoketone, one equivalent of an arylaldehyde and one equivalent of methyl-3-aminocrotonate dissolved in ethanol (˜4 equivalents) and AcOH (˜0.6 equivalent). The arylaldehyde compound used in the synthesis can be optionally substituted as desired. This mixture is, for example, stirred for 3 hours at 95° C., then cooled to ambient temperature, diluted with a solvent such as ethyl acetate, dried with a drying agent such as Na₂SO₄ and the product may be crystallized from a solvent or combination of solvents, such as ethyl acetate and hexane mixture (1:9). In the reaction below, R is a desired group such as alkyl or substituted alkyl; R⁷ is a desired group such as optionally substituted alkyl, aryl, alkoxide, or aryloxide; and R¹, R², R³, R⁴, R⁵ are independently H, alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle.

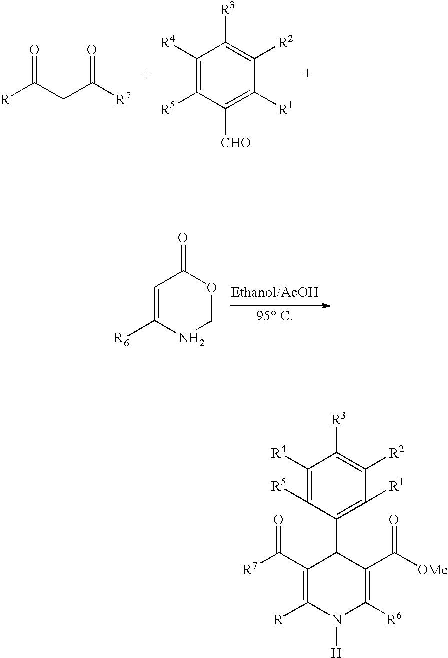

In another embodiment, 3,5 disubstituted symmetrical or unsymmetrical dihydropyridine compounds with substitution at the pyridine N are prepared by adding one equivalent of dihydropyridine to a stirring suspension of, for example, 1.5 equivalents of a metal hydride such as sodium hydride in a solvent, such as dimethylformamide (DME). The reaction mixture is stirred, for example, for 30 minutes at ambient temperature under inert, for example N₂, atmosphere. Alkyl chloride may then be added dropwise, for example, at room temperature and under N₂. After, for example, 18 hours stirring, the reaction mixture can be separated and purified, for example, by extraction. For example, the reaction mixture can added to a separatory with 50% aqueous NH₄Cl and the aqueous suspension may be extracted with ethyl acetate. The organic extract can then be washed with water, dried, for example, with Na₂SO₄, isolated, for example, by filtration, and concentrated under reduced pressure. Purification may be achieved for example by column chromatography, for example a silica gel column eluted with a solvent or solvent mixture such as 0-10% ethyl acetate and hexane (1:9). In the reaction below, R is a desired group such as alkyl or substituted alkyl; R⁶ is a desired group such as optionally substituted alkyl, aryl, alkoxide, or aryloxide; R⁷ is a desired group such as optionally substituted alkyl, aryl, alkoxide, or aryloxide; R⁸ is a desired group such as optionally substituted alkyl, aryl, alkoxide, or aryloxide; R⁹ is a desired group such as optionally substituted alkyl; and R¹, R², R³, R⁴, R⁵ are independently H, alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle.

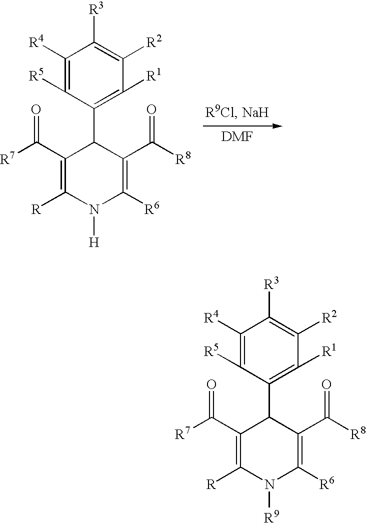

In another embodiment, 3,5 disubstituted unsymmetrical dihydropyridine compounds are prepared, for example, from ketoesters. Various protected noncommercial β-ketoesters can be synthesized, e.g., using Meldrum's acid route. The synthesis of benzylidines from ketoesters and aldehydes is accomplished, for example in 70% yield using a catalyst such as catalytic (5-10%) piperidinium acetate in alcoholic solvents at room temperature or benzene under Dean-Stark conditions. An intermediate enamide can be synthesised in situ using e.g., ammonia (THF, 30-50° C., molecular sieves 4A) or ammonium acetate (ethanol, reflux, 30 minutes). The Hantzsch reaction with benzylidines and enamides in an alcoholic solvent can result in the doubly protected C3,5-diesters. After deprotection, acid group is used to couple with different amines as required, e.g. for the synthesis of amlodipine, as shown below.

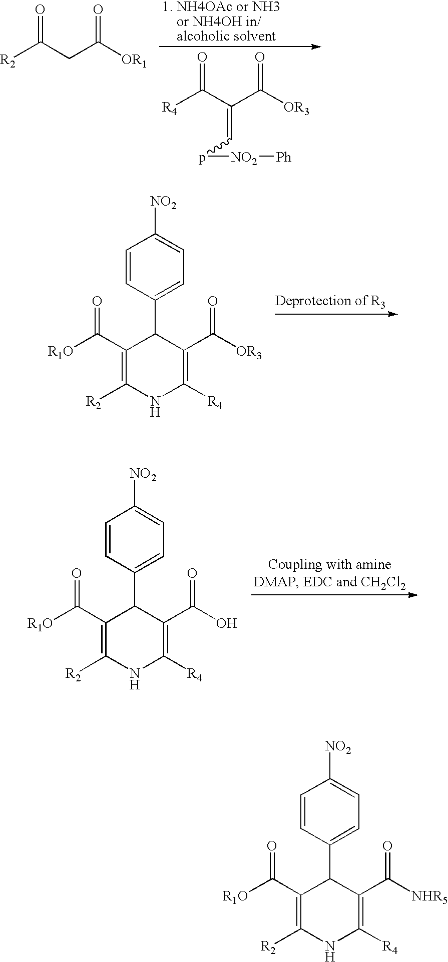

One embodiment is a solid phase method using an appropriate resin, such as Wang resin. In this method, substituted hydroxyamines are coupled to Wang resin using carbonyldiimidazole to provide 1. Treatment of 1 with 2, 2-dimethyl-6-alkyl-1,3-dioxanone at 140° C. in an inert solvent such as xylenes provides β-ketoester resin 2. Resin 2 is treated with substituted aminocrotonate, and aldehyde in DMF to form resin bound DHP 3. The resin is then washed with hydrazine (e.g. 0.5N in 1:1 EtOH:THF). Upon cleavage from resin with TFA (e.g. 25% in DCM) the desired DHP product 5 is obtained along with minor by-product which is separated, e.g., using flash chromatography, as shown below.

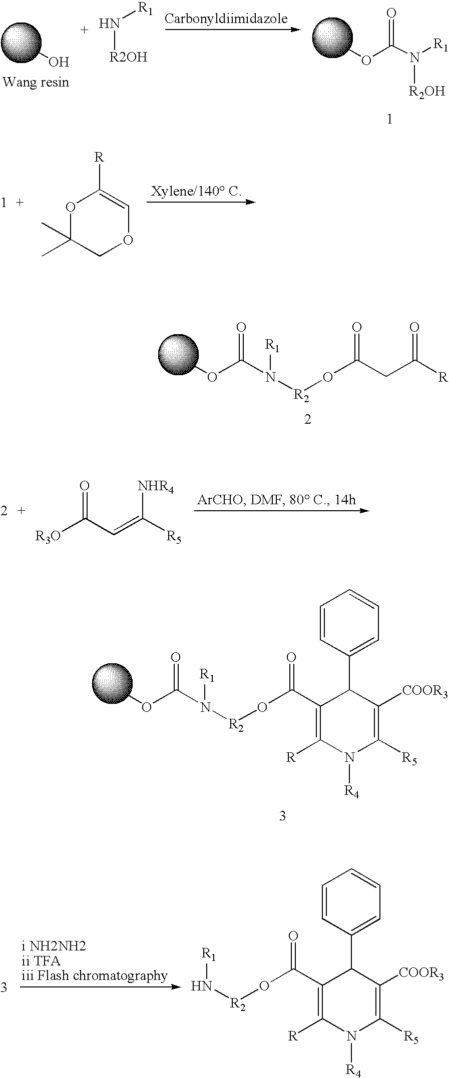

Another embodiment is the synthesis of 2-oxo-1,2-dihydropyridine, wherein differently substituted acetylenes are reacted with substituted isocyanates in presence of a catalyst, such as a Cobalt catalyst, such as n-cyclopentadienyltriphenylphosphine-2,5-diphenyl-3,4-bis-(methoxycarbonyl)cobaltacyclopentadiene in an inert solvent such as benzene and the solution is refluxed at for example 135° C. for about 1-20 hours, followed by a separation step such as flash chromatography, as shown below.

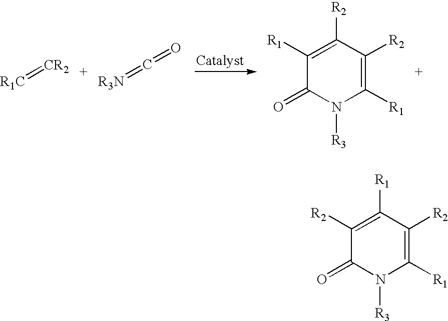

Other examples of synthetic routes which can be modified to provide the appropriate substituents are described in Examples 6-59.

Pharmaceutical Formulations and Methods of Administration

Compounds disclosed herein can be administered in an effective amount for the treatment of a disease associated with cerebral accumulation of β-amyloid, such as Alzheimer's disease, cerebral amyloid angiopathy, hereditary cerebral hemorrhage with amyloidosis Dutch-type, other forms of familial Alzheimer's disease and familial cerebral Alzheimer's amyloid angiopathy. Such compounds are also referred to herein as “active agents”. Dosage amounts and pharmaceutical formulations can be selected using methods known in the art. The compound can be administered by any route known in the art including parenteral, oral or intraperitoneal administration.

The compounds disclosed herein that are administered to animals or humans are dosed in accordance with standard medical practice and general knowledge of those skilled in the art. In particular, therapeutically effective amounts of compounds or more, can be administered in unit dosage form to animals or humans afflicted with a disease associated with cerebral accumulation of Alzheimer's amyloid or suffering from a traumatic brain injury, as well as administered diagnostically for the purpose of determining the risk of developing and/or a diagnosis of a disease associated with cerebral accumulation of Alzheimer's amyloid. In one preferred embodiment, the compound is a compound that decreases CCE, for example, by at least about 10% or more in cultured cells, and optionally reduces B amyloid production, for example, by at least about 20% or more in cultured cells that overexpress APP.

Parenteral administration includes the following routes: intravenous; intramuscular; interstitial; intra-arterial; subcutaneous; intraocular; intracranial; intraventricular; intrasynovial; transepithelial, including trnnsdermal, pulmonary via inhalation, ophthalmic, sublingual and buccal; topical, including ophthalmic, dermal, ocular, rectal, or nasal inhalation via insufflation or nebulization. The nasal inhalation is conducted, for example, using aerosols, atomizers or nebulizers.

Examples of suitable dosage amounts are, e.g., about 0.02 mg to 1000 mg per unit dose, about 0.5 mg to 500 mg per unit dose, or about 20 mg to 100 mg per unit dose. The daily dosage can be administered in a single unit dose or divided into two, three or four unit doses per day. The duration of treatment of the active agent is, for example, on the order of hours, weeks, months, years or a lifetime. The treatment may have a duration, for example, of 1-7 days, 1-4 weeks, 1-6 months, 6-12 months, or more.

The compound can be administered to the CNS, parenterally or intraperitoneally. Solutions of compound e.g. as a free base or a pharmaceutically acceptable salt can be prepared in water mixed with a suitable surfactant, such as hydroxypropylcellulose. Dispersions also can be prepared in glycerol, liquid polyethylene glycols, and mixtures thereof, and in oils. Under ordinary conditions of storage and use, these preparations can contain a preservative and/or antioxidants to prevent the growth of microorganisms or chemical degeneration.

The compounds which are orally administered can be enclosed in hard or soft shell gelatin capsules, or compressed into tablets. The compounds also can be incorporated with an excipient and used in the form of ingestible tablets, buccal tablets, troches, capsules, sachets, lozenges, elixirs, suspensions, syrups, wafers, and the like. Further, compounds can be in the form of a powder or granule, a solution or suspension in an aqueous liquid or non-aqueous liquid, or in an oil-in-water or water-in-oil emulsion.

The tablets, troches, pills, capsules and the like also can contain, for example, a binder, such as gum tragacanth, acacia, corn starch; gelating excipients, such as dicalcium phosphate; a disintegrating agent, such as corn starch, potato starch, alginic acid and the like; a lubricant, such as magnesium stearate; a sweetening agent, such as sucrose, lactose or saccharin; or a flavoring agent. When the dosage unit form is a capsule, it can contain, in addition to the materials described above, a liquid carrier. Various other materials can be present as coatings or to otherwise modify the physical form of the dosage unit. For example, tablets, pills, or capsules can be coated with shellac, sugar or both. A syrup or elixir can contain a compound as disclosed herein, sucrose as a sweetening agent, methyl and propylparabens as preservatives, a dye and flavoring. Additionally, a compound can be incorporated into sustained-release preparations and formulations.

The invention will be understood in further detail in view of the following non-limiting examples.

### EXAMPLE 1

### Measurement of Aβ1-40 and Aβ1-42

1. Materials and Methods

Chinese hamster ovary (CHO) cells, stably transfected with human APP751 (7W WT APP751 CHO cells) were used. See, e.g., Koo and Squazzo, J. Biol. Chem., Vol. 269, Issue 26, 17386-17389, Jul, 1994. The cells were maintained in DMEM medium supplemented with 10% fetal bovine serum and 1× mixture of penicillin/streptomycin/fimgizone/glutamine mixture (Cambrex, Md.) geneticin as selecting agent in 75 cm² cell culture flasks.

The 7W WT APP751 CHO cells were plated in 24-well cell culture plates in quadruplicate, containing 1 ml of culture medium, and treated with various calcium channel blocker compounds for 4 hours, 24 hours or 48 hours at 37° C. and 5% CO₂. All test compounds were diluted in dimethyl sulfoxide (DMSO) before being added to the cultured confluent 7W WT APP751 CHO cells. The culture medium was collected and diluted 5-fold for the 4 hours assay and 50-fold for the 24 hour assay before being assayed by ELISAs for Aβ1-40 and Aβ1-42, respectively. Concentrations of Aβ1-40 and Aβ1-42, expressed in pg/ml, were determined using commercially available ELISAs (Biosource, Calif.) in a colorimetric assay using labeled antibodies detected spectrophotometrically.

G-sec Inhib XIX, SKF 96365, 2-APB, felodipine, FPL, clotrimazole, tetrandrine, R24571, and econazole are available, as Calbiochem products from EMD Biosciences, Inc., La Jolla, Calif.; nilvadipine, nitrendipine and amlodipine (amlodipine besylate) are available, e.g., from Fujisawa, Osaka, Japan; thapsigargin, BAPTA-AM and TA9 (Tyrphostin A9) are available, e.g., as a Sigma product from Sigma-Aldrich Corp., St. Louis, Mo.; and felodipine, diltiazem, S(−)Bay K8644, R(+)Bay K8644, MRS 1845, SR 33805, loperamide, and isradipine are available from Tocris Cookson Inc., Ellisville, Mo.

2. Results

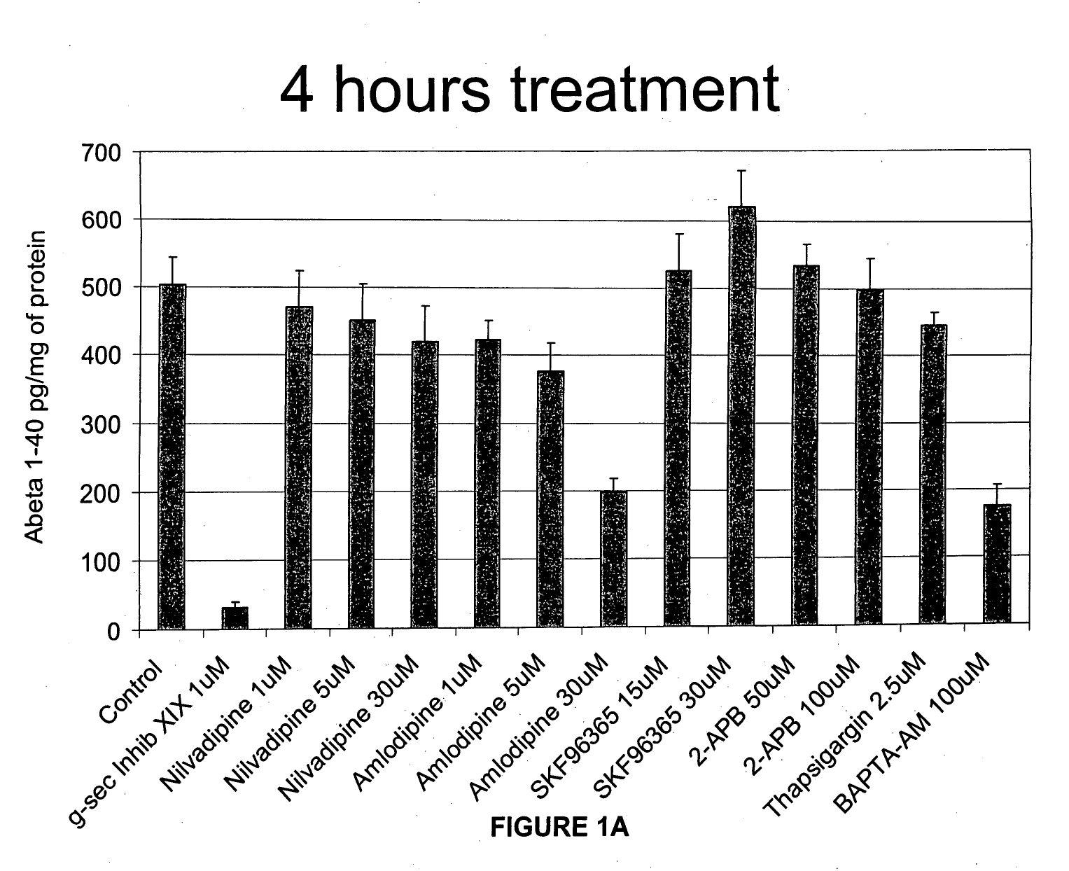

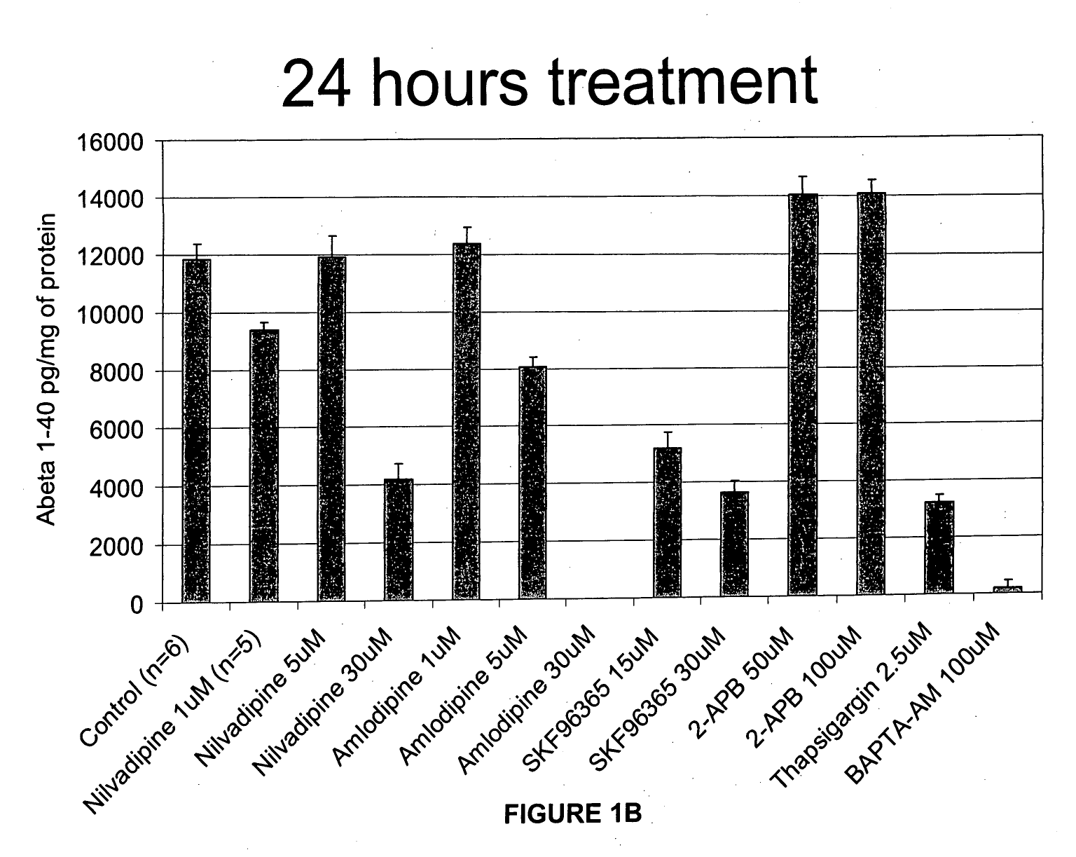

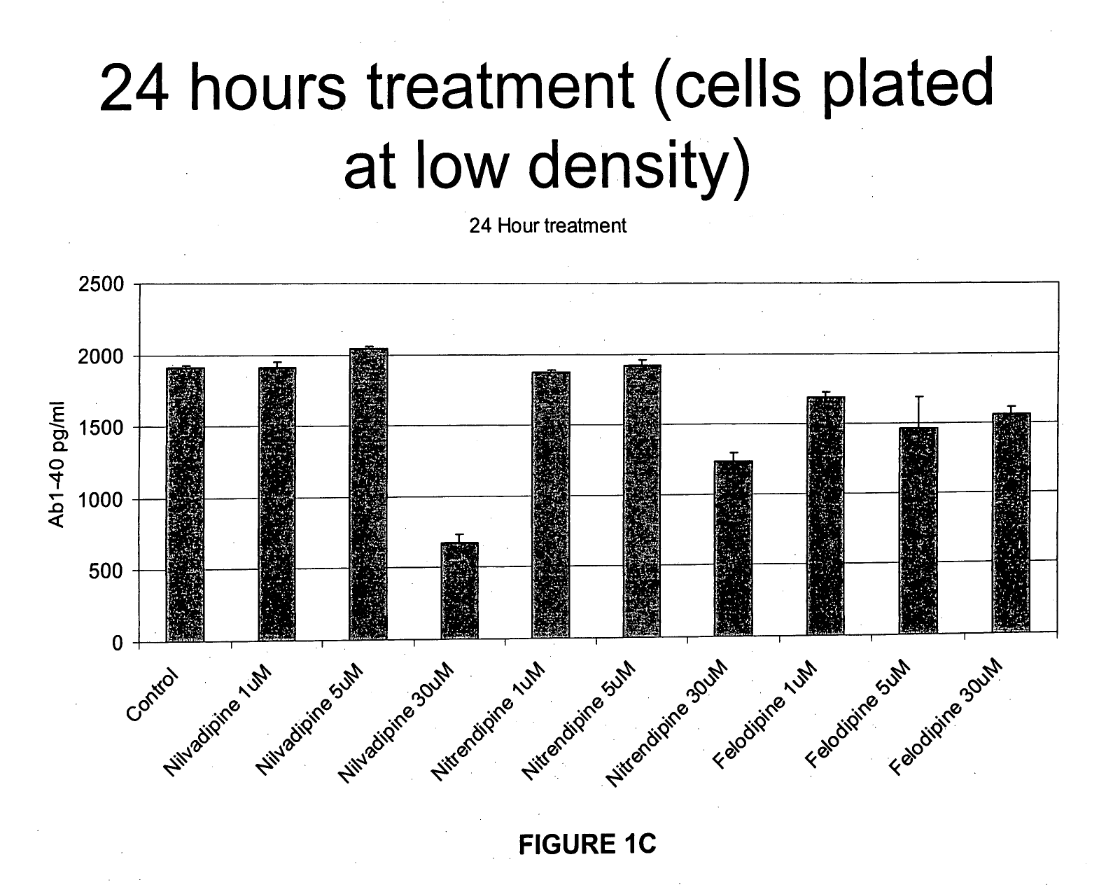

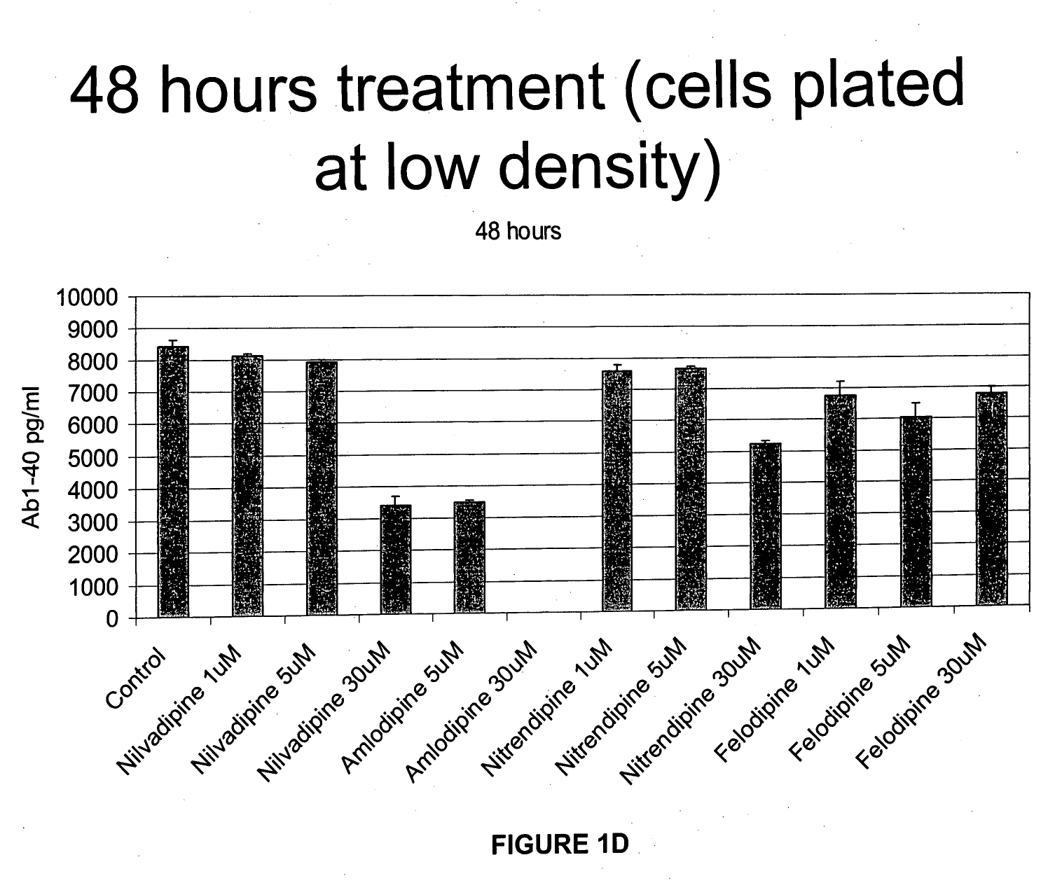

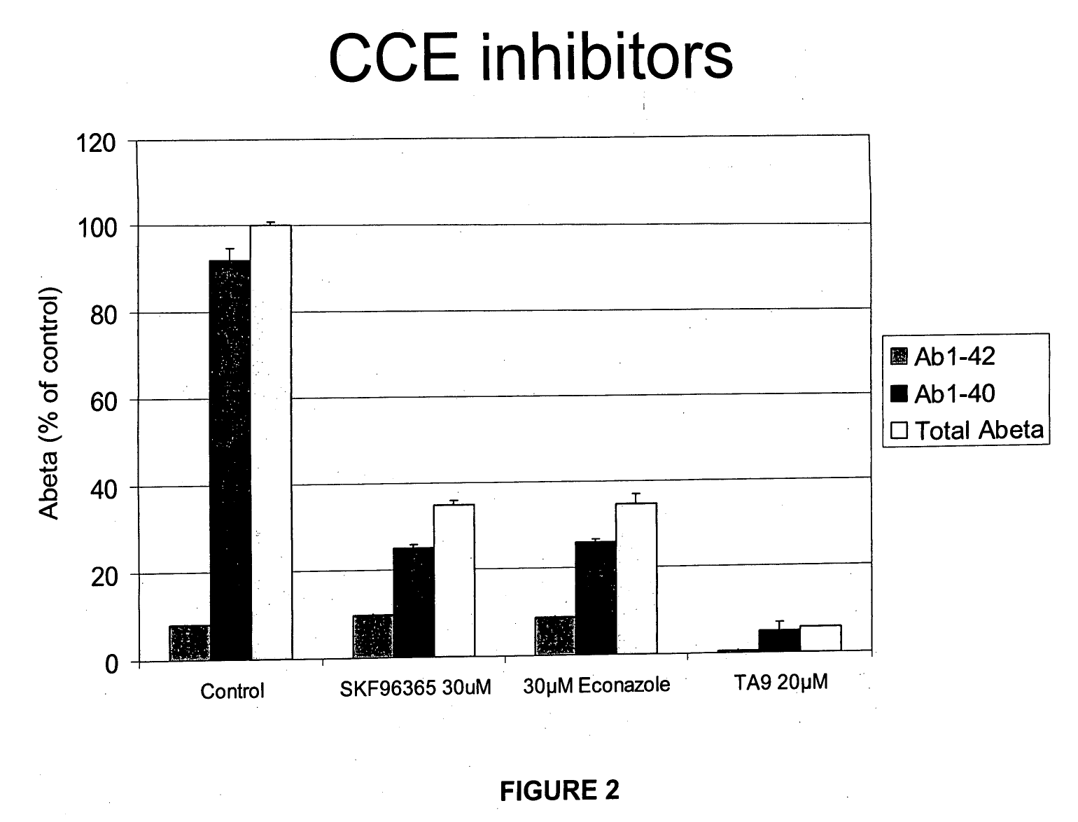

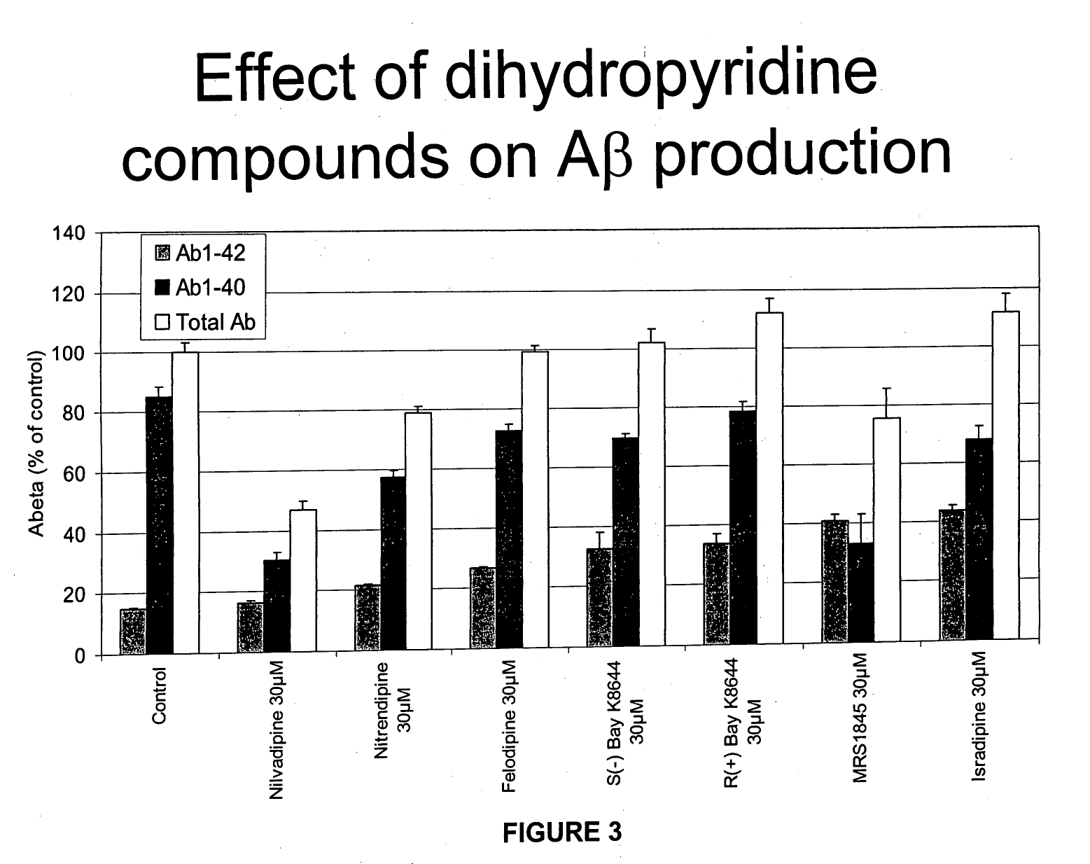

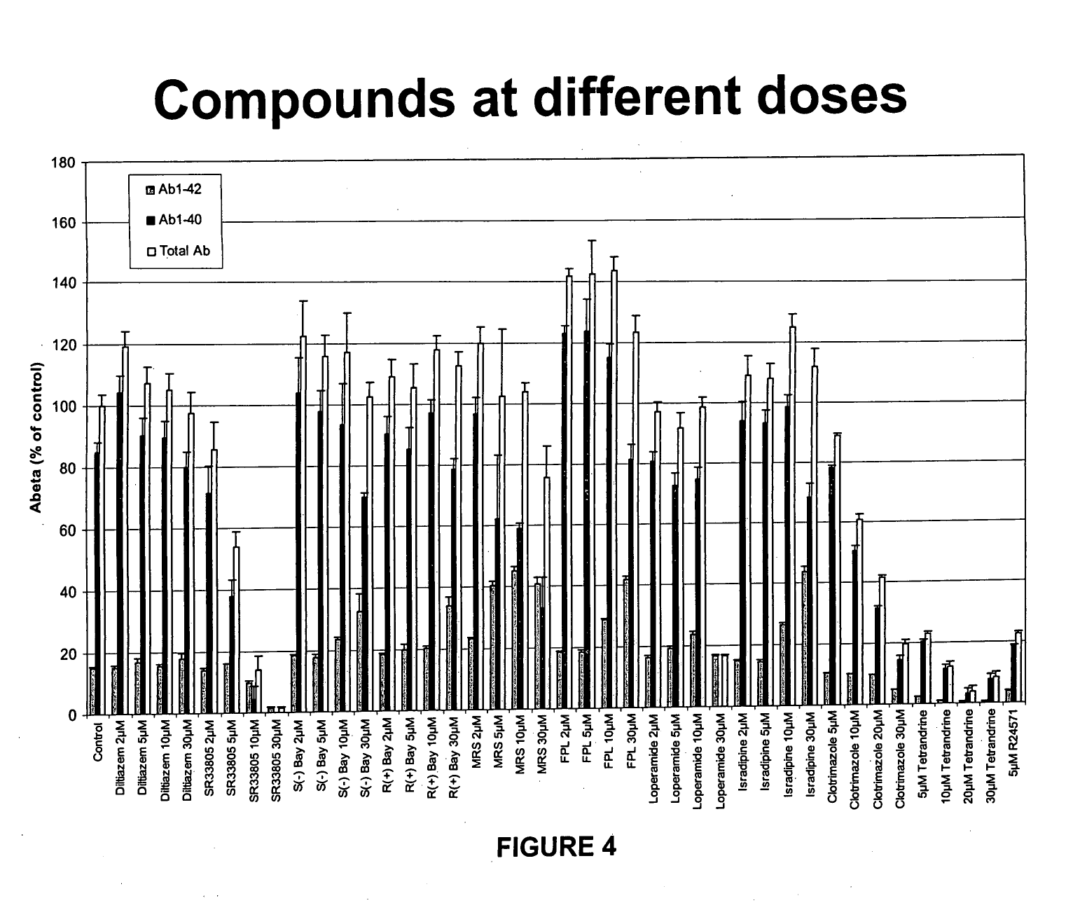

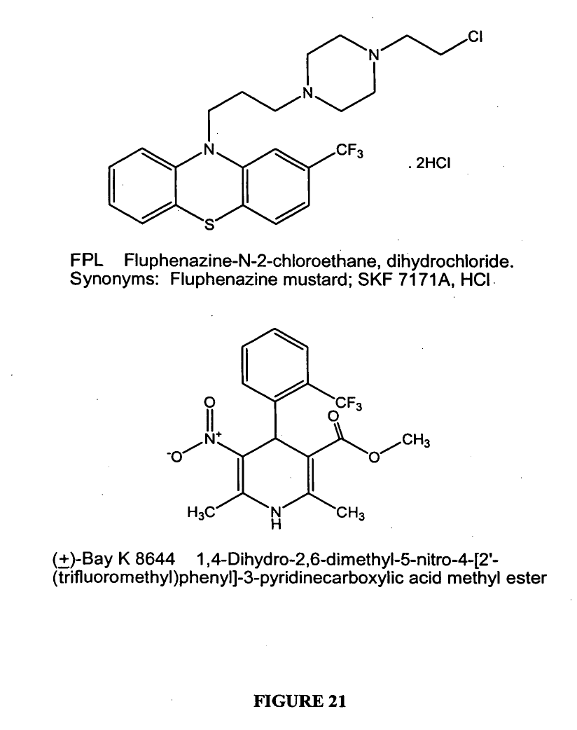

Treatment of cells with 30 μM of amlodipine for 4 hours significantly decreased the concentration of Aβ1-40 compared to controls (FIG. 1A). In FIG. 1A 2-APB refers to 2-aminoethoxydiphenylborate and BAPTA-AM refers to 1,2-Bis(2-aminophenoxy)ethane N,N,N′,N′-tetraacetic acid acetoxymethyl ester. Treatment of cells with 30 μM nilvadipine, 30 μM amlodipine, or 15 or 30 μM of SKF 96365 for 24 hours significantly decreased the concentration of Aβ1-40 compared to controls (FIG. 1B). Treatment of cells plated at low density for 24 hours with 30 μM nilvadipine or 30 μM nitrendipine for 24 hours significantly decreased the concentration of Aβ1-40 compared to controls (FIG. 1C). Treatment of cells for 48 hours plated at low density with 30 μM nilvadipine, 5 or 30 μM amlodipine, or 30 μM nitrendipine significantly decreased the concentration of Aβ1-40 compared to controls (FIG. 1D). As shown in FIG. 2, 30 μM SKF 96365, 30 μM econazole or 20 μM tyrphostin A9 (“TA9” in the Figure) significantly decreased the concentrations of Aβ1-40, Aβ1-42 and total β-amyloid compared to controls. As shown in FIG. 3, 30 μM nilvadipine, 30 μM of nitrendipine or 30 μM MRS 1845 significantly decreased the concentrations of Aβ1-40 and total β-amyloid compared to controls. As shown in FIG. 4, 10 or 30 μM SR 33805 or 30 μM of loperamide significantly decreased the concentrations of Aβ1-40, Aβ1-42 and total β-amyloid compared to controls, and 20 μM clotrimazole, 5, 10, 20 or 30 μM of tetrandine, or 5 μM R24571 significantly decreased the concentrations of Aβ1-40 and total β-amyloid compared to controls. In FIG. 4, S(−)-Bay refers to S(−)-BayK8644; R(+)-Bay refers to R(+)-Bay K8644; MRS refers to MRS 1845; and FPL refers to Fluphenazine mustard (See FIG. 21).

### EXAMPLE 2

### Screening of Dihydropylidine Compounds

1. Materials and Methods

Dihydropyridine compounds were obtained from Maybridge (England). Each compound was dissolved in DMSO. 7W WT APP751 CHO cells overexpressing APP751 were plated into 96-well culture plates in 200 μL of culture medium. Each compound from the library was added to confluent cells to a final concentration of 30 μM. After 24 hours of treatment, culture medium was collected and dissolved 10-fold and 2-fold for measuring the level of Aβ1-40 and Aβ1-42, respectively. Aβ1-40 and Aβ1-42 were determined using commercially available ELISAs (Biosource, Calif.), following the recommendations of the manufacturer.

2. Results

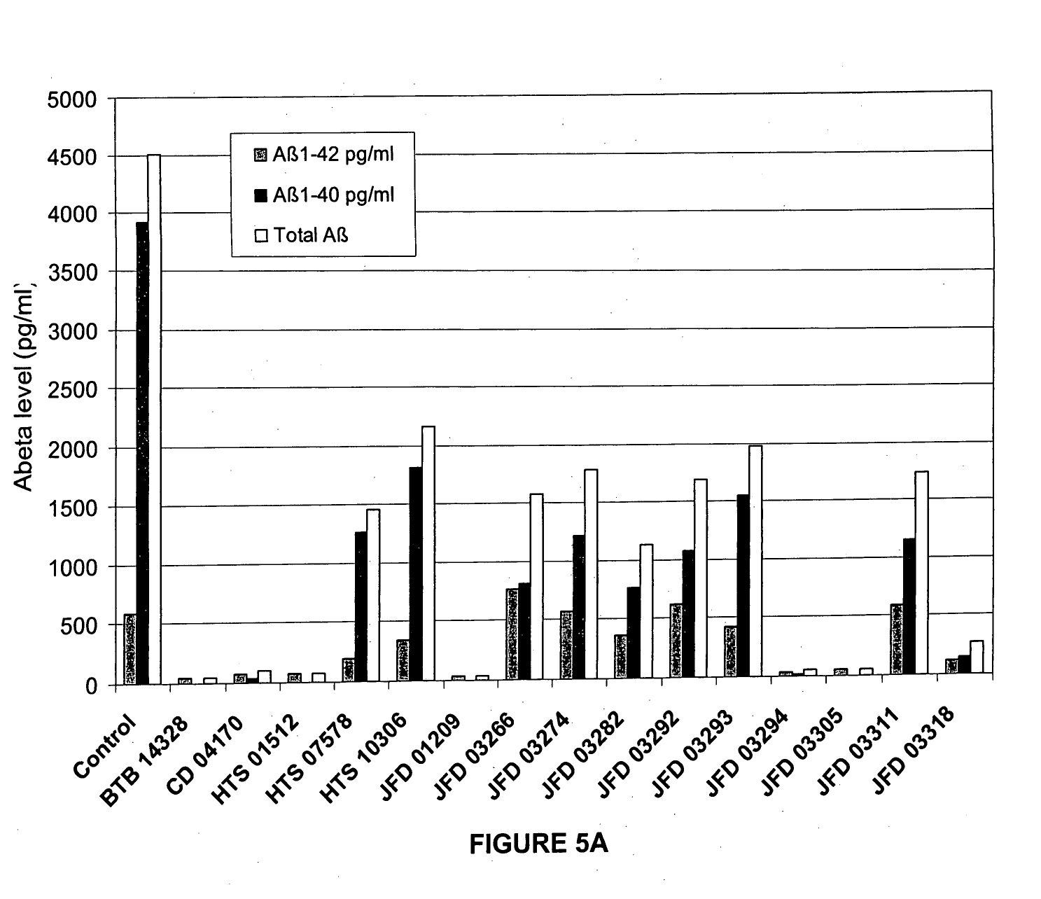

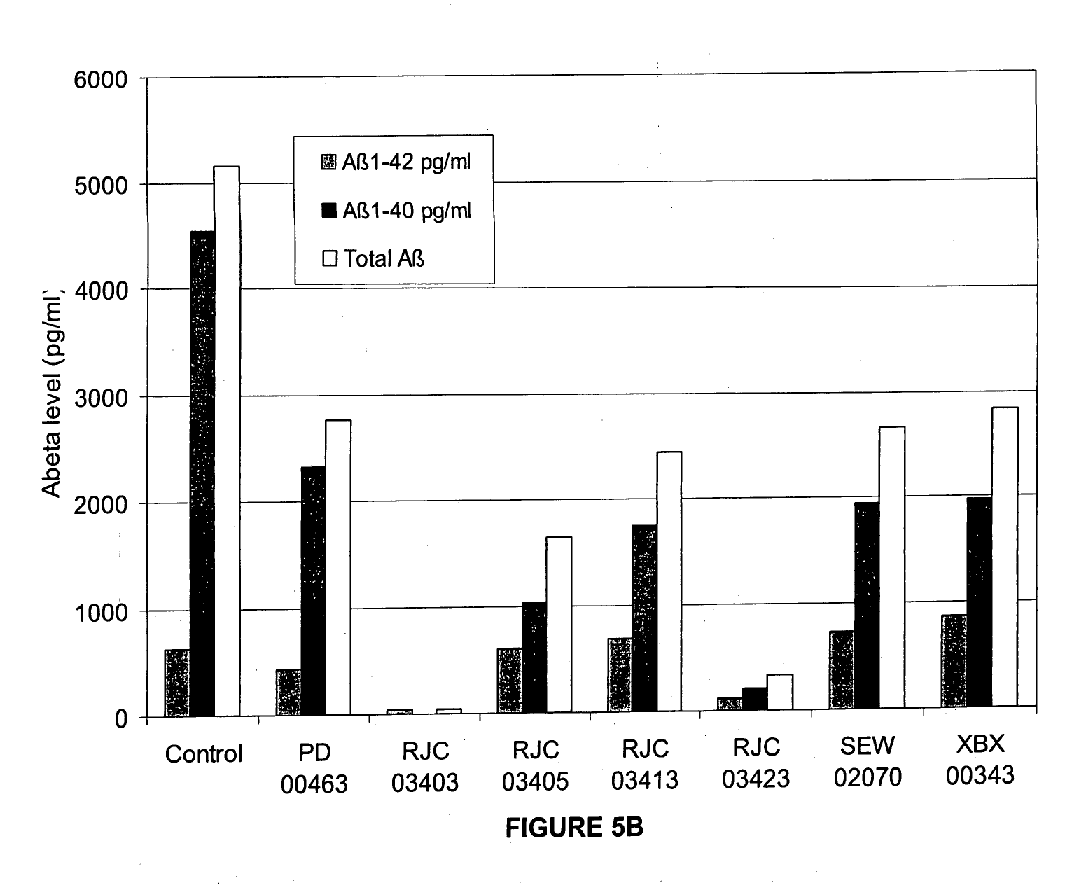

As shown in FIG. 5A, treatment of 7W WT APP751 CHO cells with 30 μM of BTB 14328, CD 04170, HTS 01512 HTS 07578, HTS 10306, JFD 01209, JFD 03282, JFD 03293, JFD 03294, JFD 03305 or JFD 03318 for 24 hours significantly decreased the concentration of Aβ1-40, Aβ1-42 and total β-amyloid (Aβ1-40+Aβ1-42) compared to controls. Treatment of 7W WT APP751 CHO cells with 30 μM of JFD 03266, JFD 03274, JFD 03292 or JFD 03311 for 24 hours significantly decreased the concentration of Aβ1-40 and total β-amyloid (Aβ1-40+Aβ1-42) compared to controls. As shown in FIG. 5B, treatment of 7W WT APP751 CHO cells with 30 μM of PD 00463, RJC 03403 or RJC 03423 for 24 hours significantly decreased the concentration of Aβ1-40, Aβ1-42 and total β-amyloid compared to controls. Treatment of 7W WT APP751 CHO cells with 30 μM of RJC 03405, RJC 03413, SEW 02070 or XBX 00343 for 24 hours significantly decreased the concentration of Aβ1-40 and total β-amyloid (Aβ1-40p+Aβ1-42) compared to controls.

### EXAMPLE 3

### Screening of NF-kB Activation Inhibitors

1. Materials and Methods

Most of the compounds screened can be obtained as Calbiochem products from EMD Biosciences, Inc., La Jolla, Calif. R— and S-Niguldipine are available e.g., from Tocris Cookson Inc., Ellisville, Mo. CAPE and Artemisinjn are available, e.g., as a Sigma product from Sigma-Aldrich Corp., St. Louis, Mo.

Each compound was dissolved in DMSO. 7W WT APP751 CHO cells overexpressing APP751 were plated into 96-well culture plates in 200 μL of culture medium. Each compound from the library was added to confluent cells to a final concentration of 500 nM, 1 μM, 5 μM, 10 μM and/or 30 μM. After 24 hours of treatment, culture medium was collected and dissolved 10-fold and 2-fold for measuring the level of Aβ1-40 and Aβ1-42, respectively. Aβ1-40 and Aβ1-42 were determined using commercially available ELISAs (Biosource, Calif.), following the recommendations of the manufacturer.

2. Results

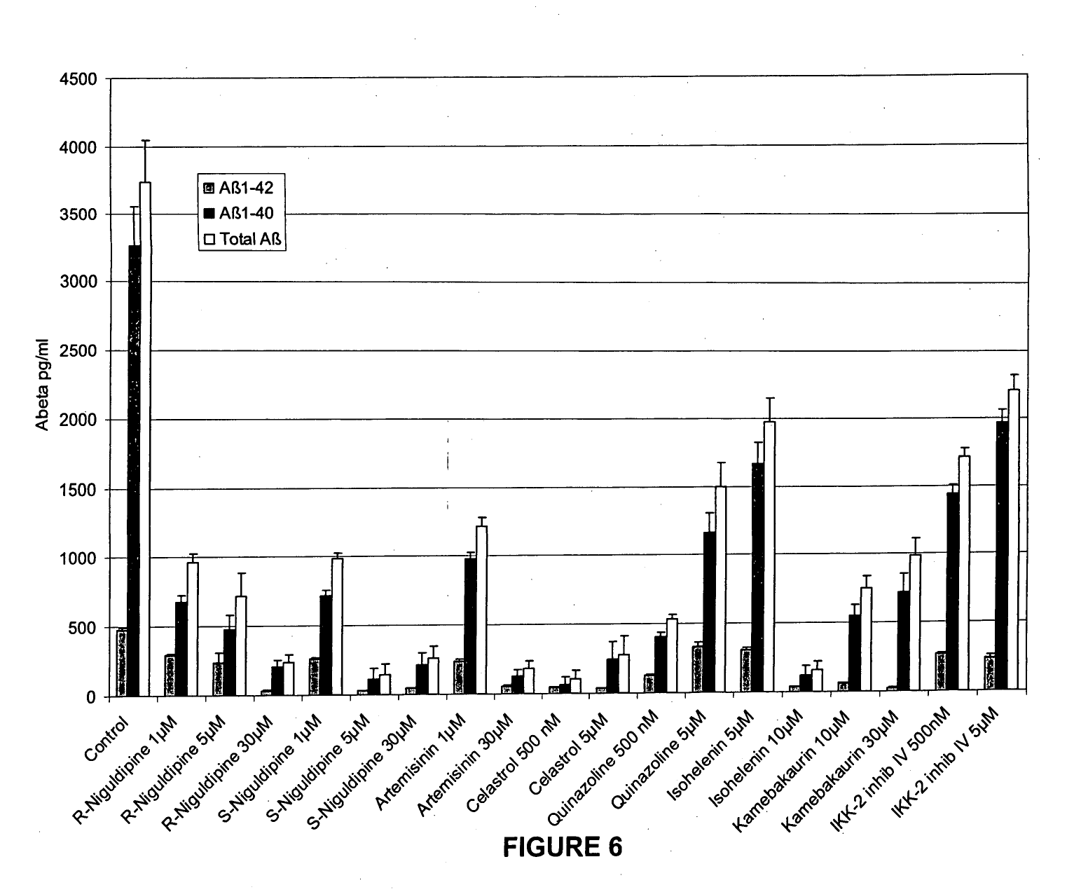

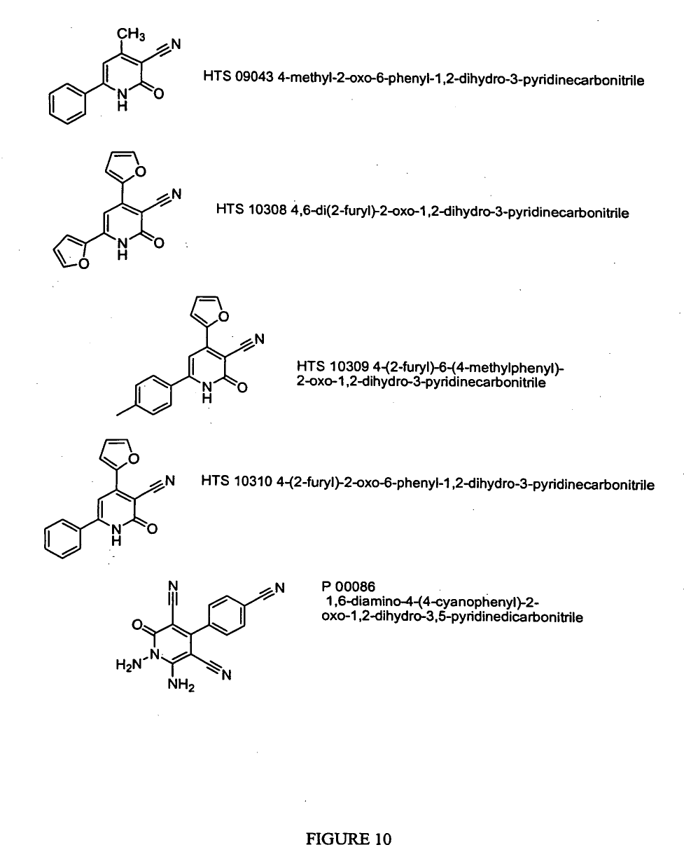

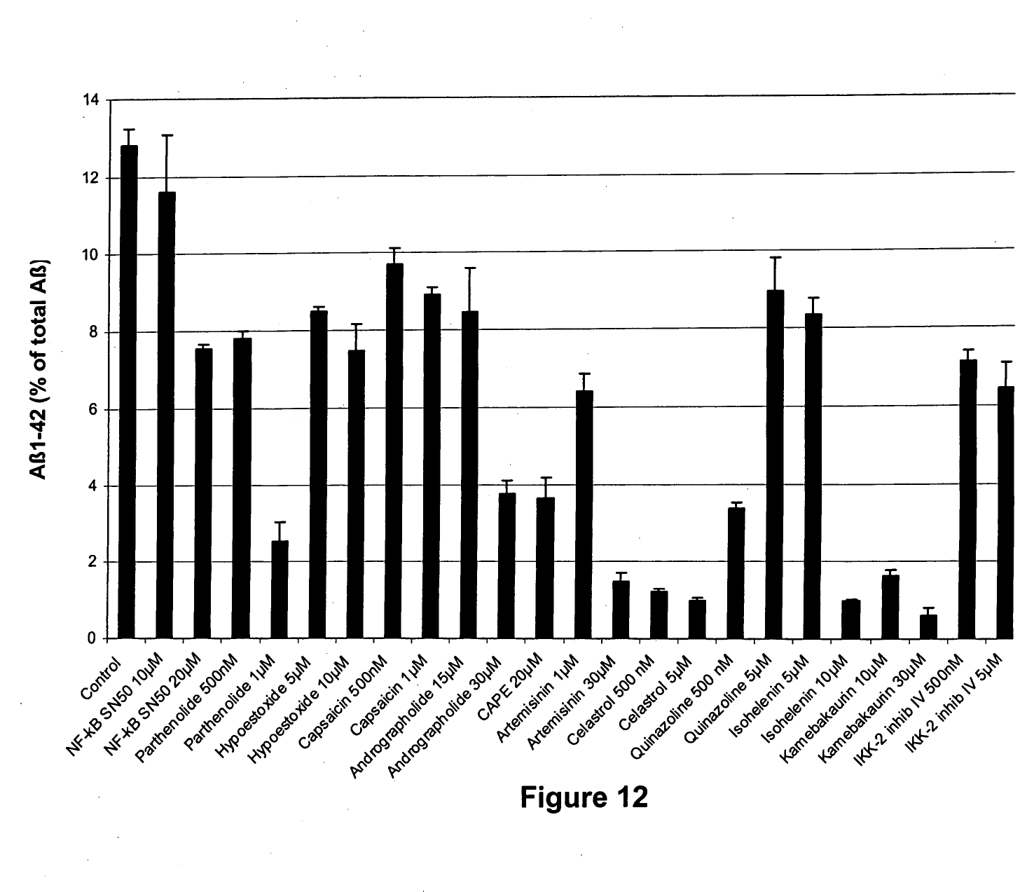

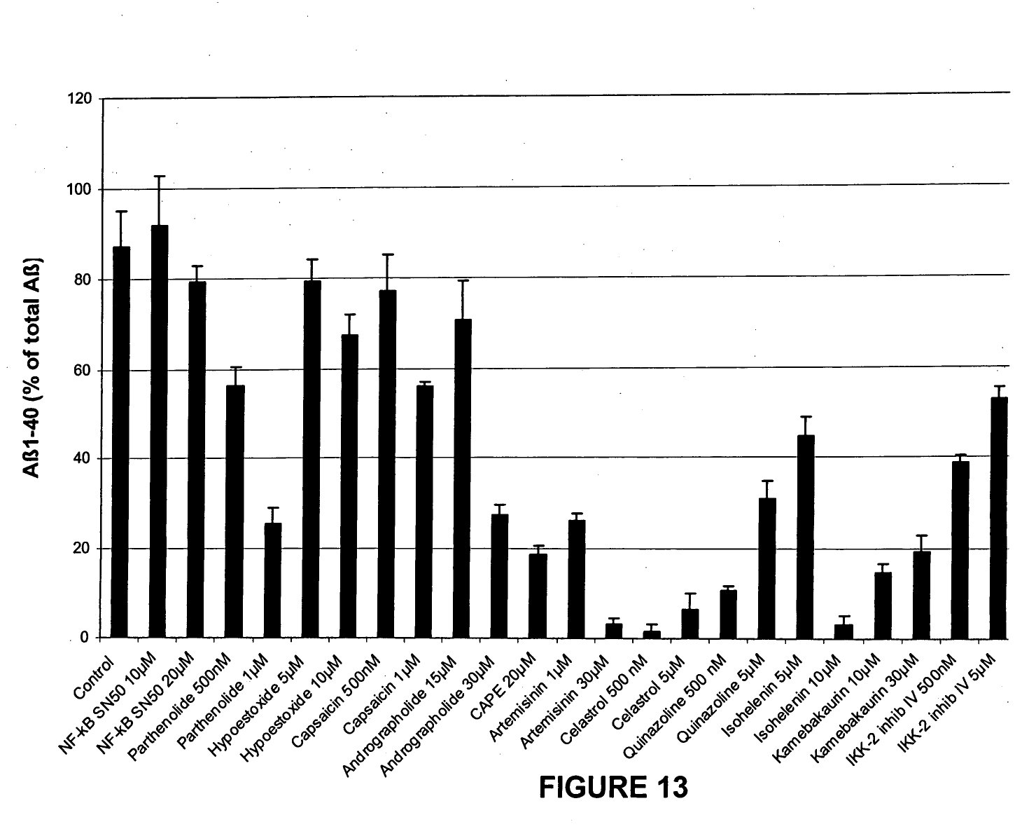

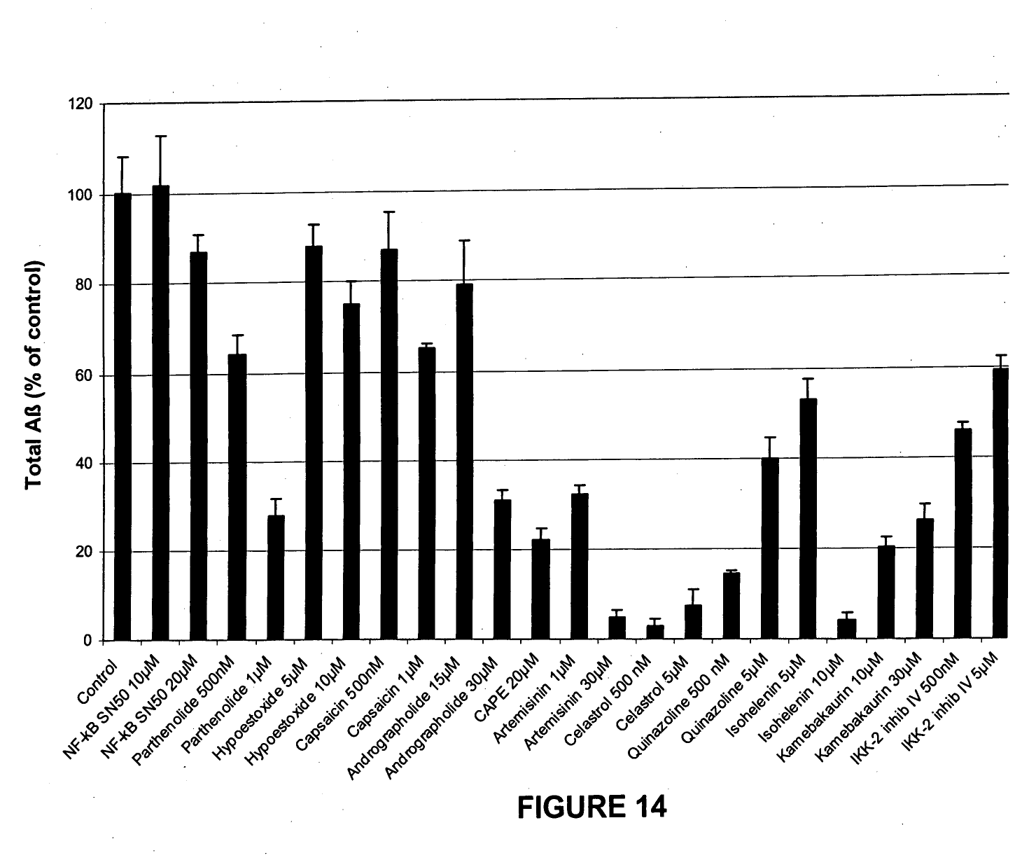

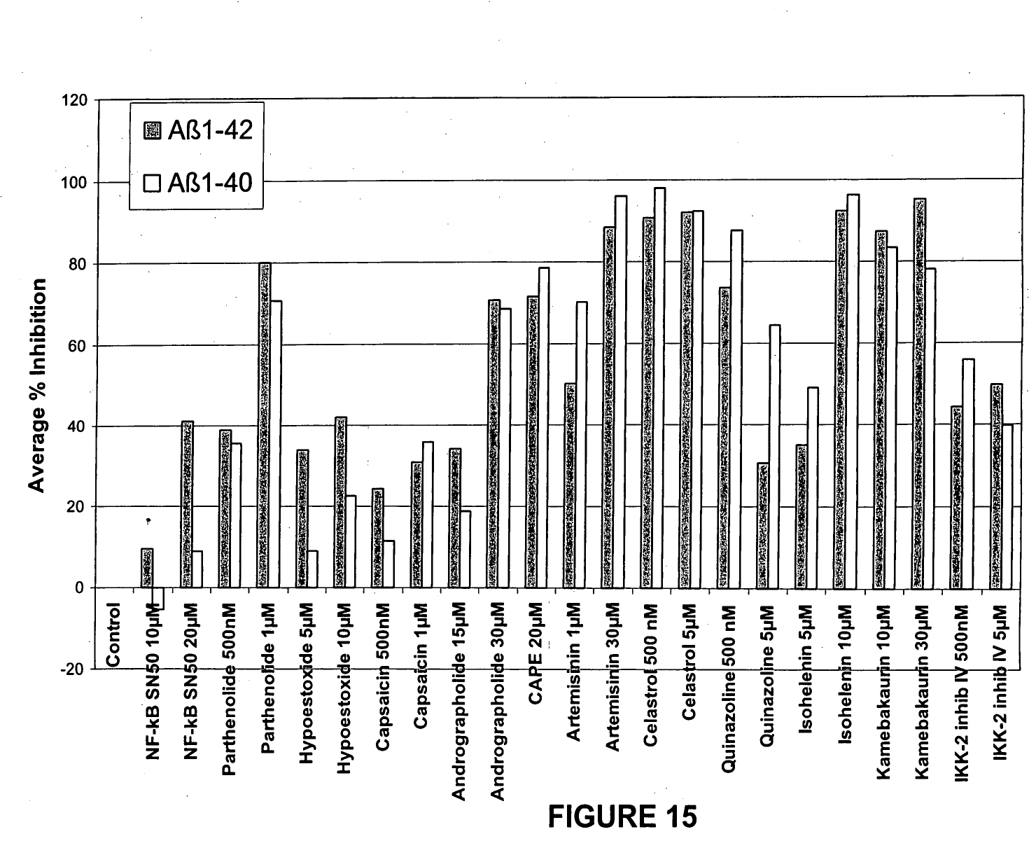

As shown in FIG. 6, treatment of 7W WT APP751 CHO cells with 1, 5 or 30 μM R-niguldipine, 1, 5 or 30 μM (S)-(+)-niguldipine, 1 or 30 μM artemisinin, 500 nM or 5 μM celastrol, 500 nM or 5 μM of the NF-kb activation inhibitor, 6-amino-4-(4-phenoxyphenylethylamino)quinazoline, referred to as “quinazoline” in the FIG. 5 or 10 μM isohelenin, 10 or 30 μM kamebakaurin, or 500 nM or 5 μM IKK-2 Inhibitor IV for 24 hours significantly decreased the concentration of Aβ1-40, Aβ1-42 and total β-amyloid compared to controls. Further results in additional runs with additional compounds are shown in FIGS. 12-15.

### EXAMPLE 4

### Capacitative Calcium Entry Assay

CCE activity was assayed by calcium fluorometric measurements using microplates. In particular, Chinese hamster ovary cells (7W WT APP751 CHO cells) overexpressing APP were grown on 96 well assay plates (sterile black plate, clear bottom with lid, tissue culture treated, Costar ref# 3603) for 24 hours in DMEM medium (Gibco, Invitrogen corporation) containing 10% serum. Fluo-4 acetoxymethyl ester (Fluo-4/AM ester; special FluoroPure™ grade with >98% HPLC purity specification, Molecular Probes, OR, ref# F-23917) was dissolved in DMSO and further solubilized in DMEM medium to a concentration of 10 μM. Confluent CHO cells then were washed with fresh DMEM and incubated with 200 μL of Fluo4/AM (dissolved in DMEM) for 30 minutes at 27° C. After this incubation period, cells were washed with 200 μL of HBSS (145 mM NaCl, 2.5 mM KCl, 1 mM MgCl₂, 20 mM HEPES, 10 mM glucose) containing 500 μM EGTA and immediately washed 3 times with 200 μL of HBSS, using a multi-channel micropipette. Cells then were incubated (and protected from light) in 100 μL of HBSS (free of calcium) for 30 minutes at 27° C.

After this incubation period, the microplate containing the cells was loaded with the different compounds to be tested and immediately inserted into a spectrofluorometer (Synergy HTTR (Bio-Tek, Vt., USA)) equipped with 2 microinjectors with a computer interface and thermoregulated at 27° C. The first microinjector of the spectrofluorometer was loaded with HBSS containing 4.5 μM thapsigargin (TG), whereas the second microinjector was loaded with HBSS containing 8 mM CaCl₂. The spectrofluorometer was programmed to read each well of the plate using the kinetic mode. Each read was done by using the following parameters: excitation at 485 nm and emission at 516 nm. First, 11 reads with an interval of 1 minute and 25 seconds between each read were performed to determined the baseline fluorescence. Then, 50 μL of HBSS containing 4.5 μM TG (delivered at a speed of 300 μL/second) was added to all the wells of the microplate (final concentration of TG: 1.5 μM). One minute and 25 seconds after TG was added, 11 reads (with an interval of 1 minute and 25 seconds between each read) were performed, then 50 μL of HBSS containing 8 mM CaCl₂ was added to each well (final calcium concentration of 2 mM) and 11 reads (with an interval of 1 minute and 25 second between each read) were performed. The peak amplitude of CCE was determined by subtracting the fluorescent value obtained during the reading number 23 by the fluorescent value obtained during the reading number 22.

For each compound tested, experiments were replicated eight times and the mean peak amplitude of CCE was calculated for each compound. For each plate, 8 wells were used as controls to determine the mean peak amplitude of CCE in untreated cells. The percentage CCE inhibition was calculated according to the following formulae: 100*(A−B)/A, where A represents the mean peak amplitude of CCE in untreated cells (control) and B the mean peak amplitude of CCE in treated cells.

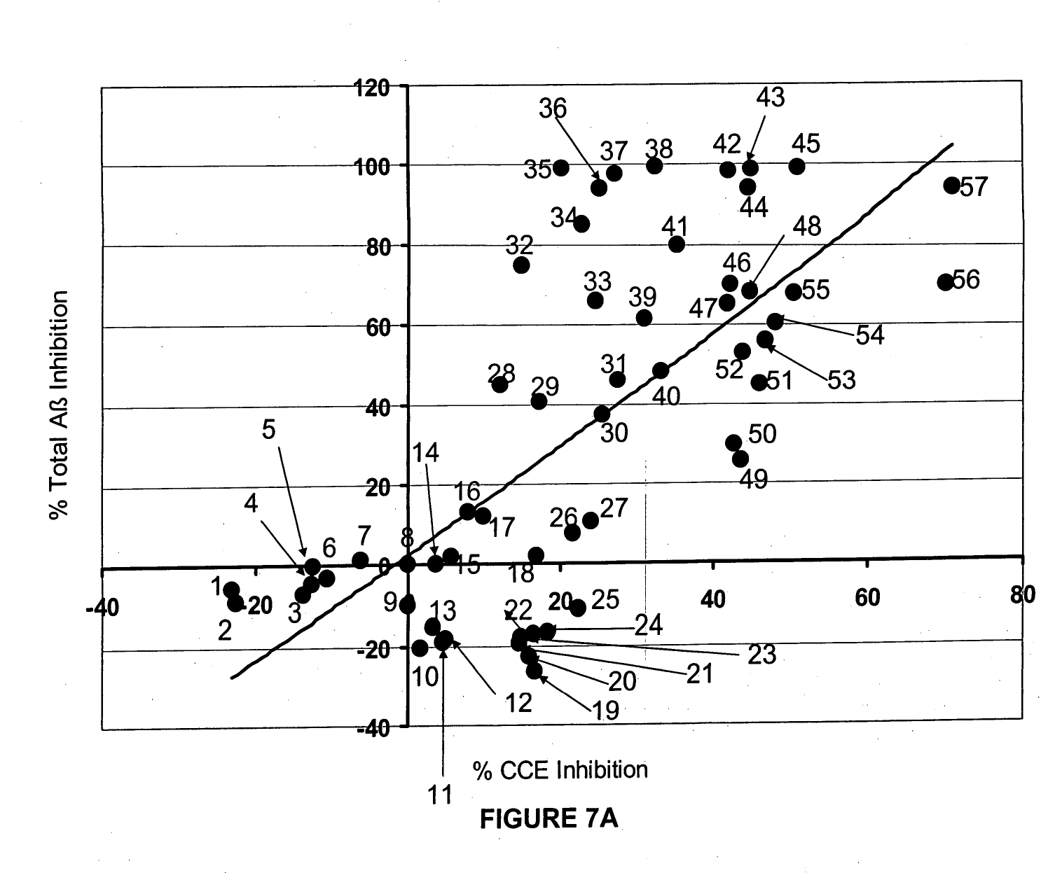

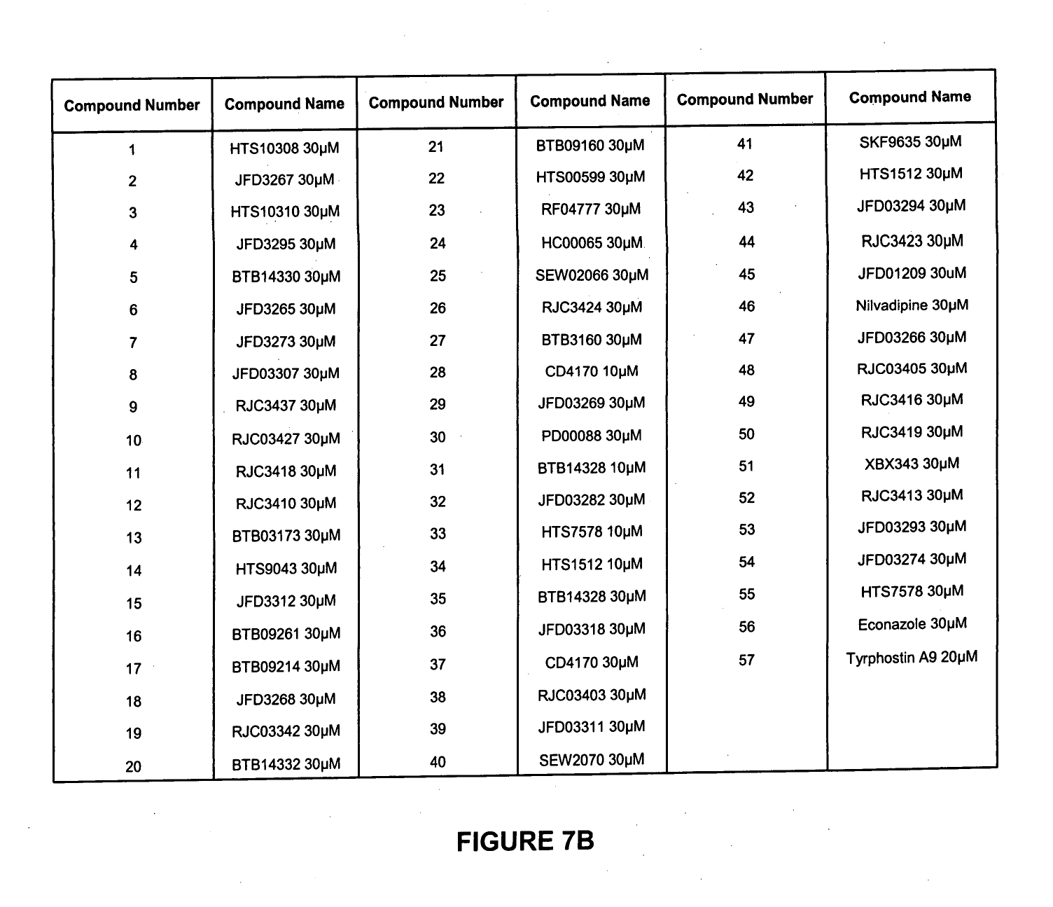

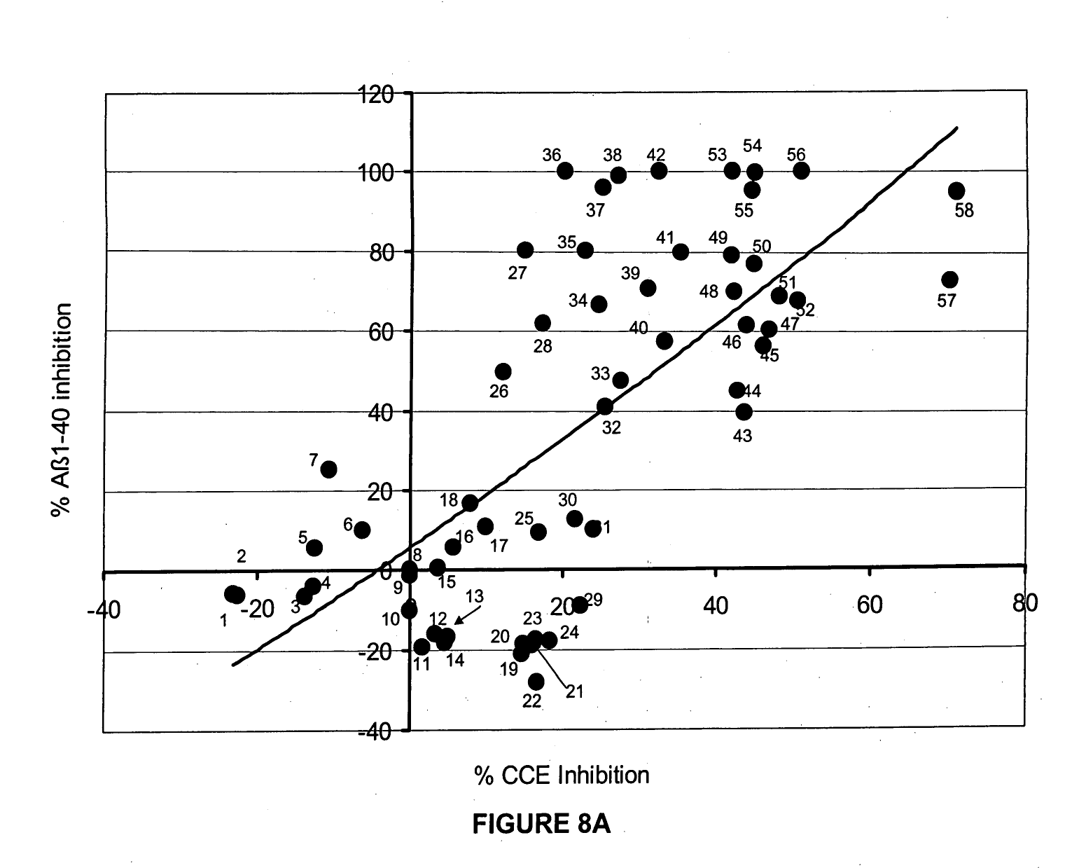

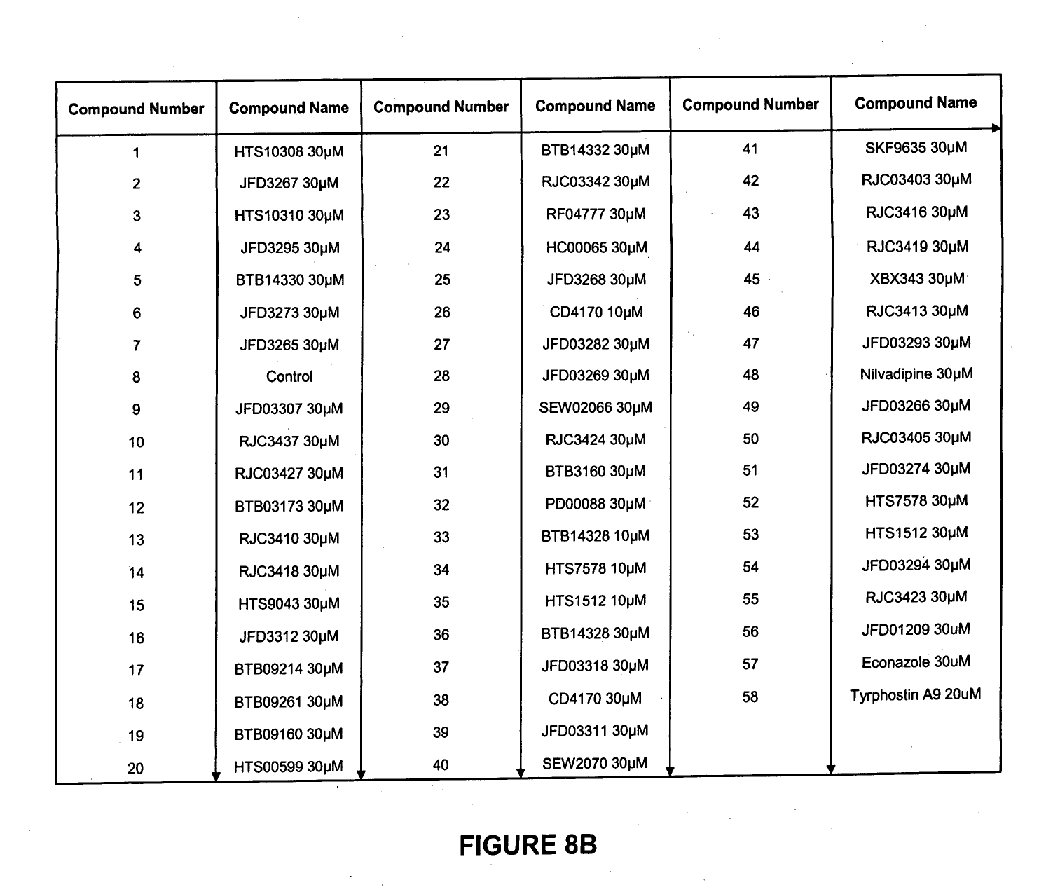

Compounds which inhibited CCE in the CHO cells also inhibited, i.e., decreased, total Aβ production as shown in FIG. 7A (a correlation graph for CCE inhibition and total β-amyloid inhibition, FIG. 7B (list of compounds shown in FIG. 7A), FIG. 8A (correlation of % CCE inhibition and % Aβ1-40 inhibition) and FIG. 8B (list of compounds shown in FIG. 8A). With the following exceptions, the compounds shown in FIG. 8B are all available, e.g., from Maybridge plc, Cornwall, England. SKF96365 and Econazole are available, e.g., as Calbiochem products from EMD Biosciences, Inc., La Jolla, Calif. Nilvadipine is available, e.g., from Fujisawa, Osaka, Japan. Tyrphostin A9 is available, e.g., as a Sigma product from Sigma-Aldrich Corp., St. Louis, Mo.

See also FIGS. 16-20 where for compounds obtained from Maybridge plc, Cornwall, England the Maybridge compound name is used.

### EXAMPLE 5

### Screening of Dihydropyridine Compounds

1. Materials and Methods

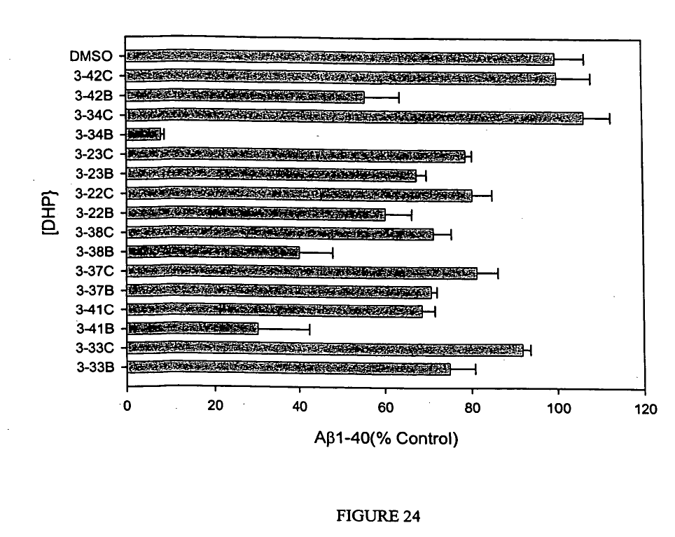

The screening of dihydropyridine compounds was conducted according to the procedure described in Example 1. Compounds 2-19, 2-32, 2-23, 2-33, 2-27, 2-28, and 2-29, as shown in Table 2, were tested. Each compound was added to confluent cells to a final concentration of 3, 10, 30 or 100 μM and tested. Compounds 3-42, 3-34, 3-23, 3-22, 3-38, 3-37, 341, and 3-33, as shown in Table 2, were also tested. Each of these compounds was added to confluent cells to a final concentration of 3 μM (noted as “C” in FIG. 24) or 10 μM (noted as “B” in FIG. 24).

2. Results

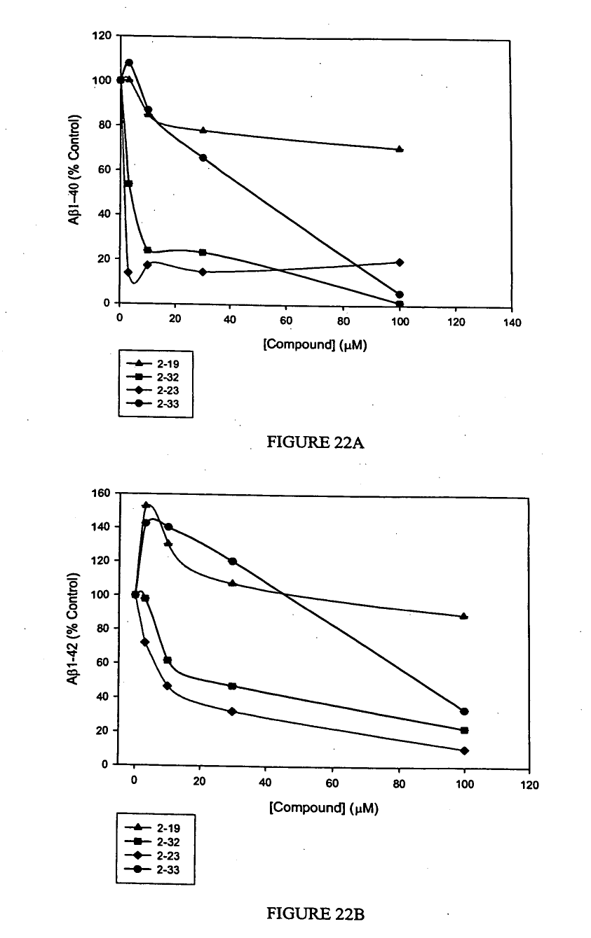

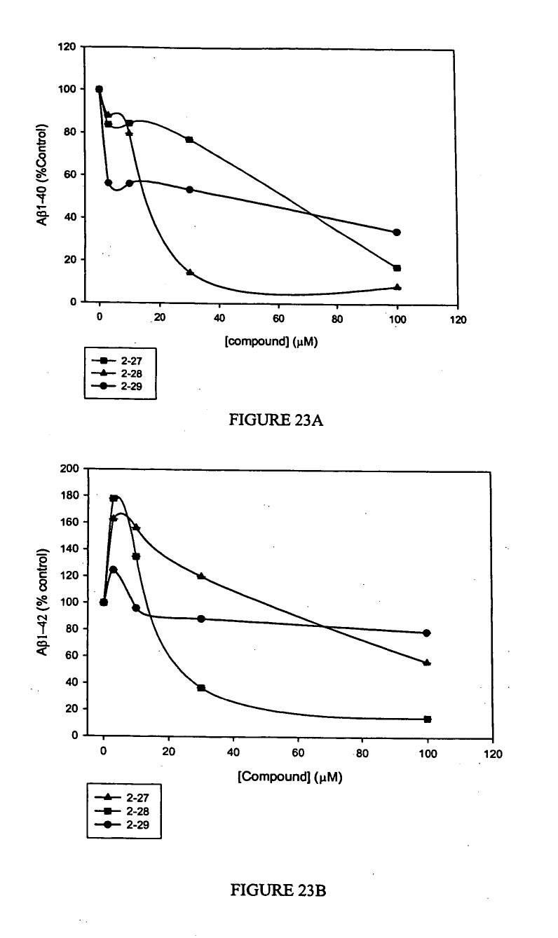

The results of treatment of 7W WT APP751 CHO cells with 3, 10, 30 and 100 μM of each of compounds 2-19, 2-32, 2-23, 2-33, 2-27, 2-28, and 2-29, for 24 hours, on the production of Aβ1-40 and Aβ1-42 are shown in FIGS. 22A, 22B, 23A, 23B. The compounds decreased the concentration of Aβ1-40 or Aβ1-42 compared to control.

The results of treatment of 7W WT APP751 CHO cells with 3 and 10 μM of each of compounds 342, 3-34, 3-23, 3-22, 3-38, 3-37, 341, and 3-33, for 24 hours, on the production of Aβ1-40 are shown in FIG. 24. The compounds decreased the concentration of Aβ1-40 compared to control.

General Techniques for Examples 6-59

All reactions requiring anhydrous conditions were conducted in oven-dried glass apparatus under an atmosphere of nitrogen. Preparative chromatographic separations were performed on Combiflash Companion, Isco Inc.; reactions were followed by TLC analysis using silica plates with fluorescent indicator (254 mn) and visualized with UV, phosphomolybdic acid or 4-hydroxy-3-methoxybenzaldehyde. All commercially available reagents were purchased from Aldrich and Acros and were typically used as supplied.

Melting points were recorded using open capillary tubes on a Barnstead melting point apparatus and are uncorrected. ¹H and ¹³C NMR spectra were recorded in Fourier transform mode at the field strength specified on a Varian AS500 spectrometer. Spectra were obtained on CDCl₃ solutions in 5 mm diameter tubes, and the chemical shift in ppm is quoted relative to the residual signals of chloroform (δ_{H} 7.25 ppm, or δ_{C} 77.0 ppm). Multiplicities in the 1H NMR spectra are described as: s=singlet, d=doublet, t=triplet, q=quartet, m=multiplet, br=broad; coupling constants are reported in Hz. Low (MS) resolution mass spectra were measured on a Micromass Q-T of API-US spectrometer utilizing an Advion Bioscience Nanomate electrospray source. Ion mass/charge (m/z) ratios are reported as values in atomic mass units.

### EXAMPLE 6

### Diethyl 4-(2-chlorophenyl)-1,4-dihydro-2,6-dimethylpyridine-3,5-dicarboxylate

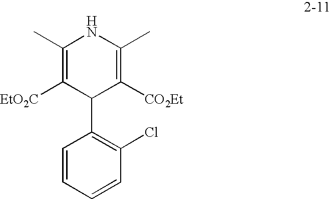

Ethyl acetoacetate (25.3 mL, 99%, 200 mmol) and 2-chlorobenzaldehyde (11.3 mL, 99%, 100 mmol) were taken up in EtOH (20 mL) at room temperature (rt). NH₄OH (10 mL) was added, the mixture was stirred at rt for 1 h, then the mixture was heated to 100° C. After 3 h, the reaction mixture was cooled to ambient temperature, azeotroped with toluene and crystallized from hot hexane to afford 9.63 g (26%) of diethyl 4-(2-chlorophenyl)-1,4-dihydro-2,6-dimethylpyridine-3,5-dicarboxylate as a white solid: MP 120-121° C.; 1H NMR (500 MHz, CDCl3) δ 1.20 (t, j=7.0 Hz, 6H), 2.31 (s, 6H), 4.04-4.11 (m, 4H), 5.40 (s, 1H), 5.61 (brs, 1H), 7.04 (t, J=7.5 Hz, 1H), 7.12 (t, J=8.0 Hz, 1H), 7.23 (d, J=8.0 Hz, 1H), 7.38 (d, J=7.5 Hz, 1H); 13C NMR (125 MHz, CDCl3) δ 14.3, 19.6, 37.5, 59.7, 103.9, 126.7, 127.3, 129.3, 131.6, 132.5, 143.7, 145.6, 167.6; MS (ES) m/z 386 (M+Na)+, 364 (M+H)+, 318, 291, 272, 252; m/z 363.112 (calcd for C₁₉H₂₂CNO₄: 363.124).

### EXAMPLE 7

### 4-(2-Chlorophenyl)-1,4-dihydro-2,6-dimethylpyridine-3,5-di(2-ethanone)

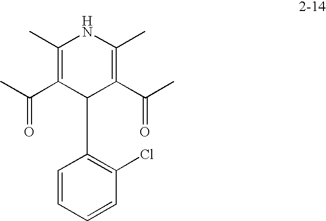

2,4-Pentanedione (1.03 mL, 99%, 10.0 mmol) and 2-chlorobenzaldehyde (562 L, 99%, 5.00 mmol) were taken up in EtOH (1 mL) at rt. NH₄OH (500 μL) was added, the mixture was stirred at rt 1 h, then the mixture was heated to 100° C. After 3 h, the reaction mixture was cooled to ambient temperature, azeotroped with toluene and crystallized from EtOAc/hexane (2:3) to afford 231 mg (15%) of 4-(2-chlorophenyl)-1,4-dihydro-2,6-dimethylpyridine-3,5-di(2-ethanone) as a pale yellow solid: MP 196-197° C.; 1H NMR (500 MHz, CDCl₃) δ 2.26 (s, 6H), 2.31 (s, 6H), 5.43 (s, 1H), 5.73 (brs, 1H), 7.08 (t, J=7.5 Hz, 1H), 7.14 (t, J=7.5 Hz, 1H), 7.25-7.28 (m, 2H); 13C NMR (125 MHz, CDCl3) δ 19.9, 30.0, 38.5, 113.5, 127.7, 128.2, 129.8, 130.7, 141.3, 143.8, 199.3; MS (ES) m/z 629 (2M+Na)+, 304 (M+H)+, 193; m/z 304.064 (calcd for C₁₇H₁₉ClNO₂ (M+H)+: 304.110).

### EXAMPLE 8

### Dimethyl 4-(2-chlorophenyl)-1,4-dihydro-2,6-dimethylpyridine-3,5-dicarboxylate

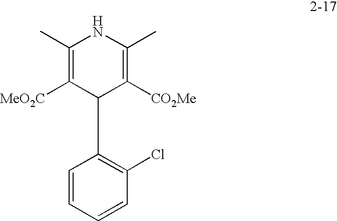

Methyl acetoacetate (1.08 mL, 99+%, 10.0 mmol) and 2-chlorobenzaldehyde (562 μL, 99%, 5.00 mmol) were taken up in EtOH (1 mL) at rt. NH₄OH (500 μL) was added, the mixture was stirred at rt 1 h, 75° C. 1 h, then the mixture was heated to 95° C. After 2 h, the reaction mixture was cooled to ambient temperature, azeotroped with toluene and crystallized from EtOAc/hexane (1:5) to afford 760 mg (45%) of dimethyl 4-(2-chlorophenyl)-1,4-dihydro-2,6-dimethylpyridine-3,5-dicarboxylate as a white solid: MP 188-189° C.; ¹H NMR (500 MHz, CDCl₃) δ 2.32 (s, 6H), 3.61 (s, 6H), 5.40 (s, 1H), 5.65 (brs, 1H), 7.04 9t, J=8.0 Hz, 1H), 7.13 (t, J=7.5 Hz, 1H), 7.23 (d, J=8.0 Hz, 1H), 7.37 (d, J=7.5 Hz, 1H); ¹³C NMR (125 MHz, CDCl₃) δ 19.4, 37.2, 50.8, 104.0, 126.9, 127.3, 129.3, 131.2, 132.4, 144.0, 145.9, 168.0; MS (ES) m/z 693 (2M+Na)⁺, 358 (M+Na)⁺, 336 (M+H)⁺, 304, 272, 224; m/z 336.089 (calcd for C₁₇H₁₉ClNO₄ (M+H)⁺: 336.100)

### EXAMPLE 9

### Di-tert-butyl 4-(2-chlorophenyl)-1,4-dihydro-2,6-dimethylpyridine-3,5-dicarboxylate

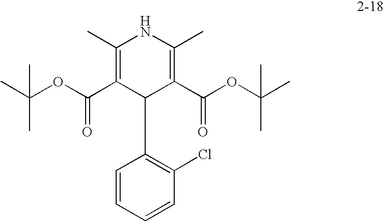

tert-Butyl acetoacetate (1.65 mL, 99%, 10.0 mmol) and 2-chlorobenzaldehyde (562 μL, 99%, 5.00 mmol) were taken up in EtOH (1 mL) at rt. NH₄OH (500 μL) was added, the mixture was stirred at rt 1 h, 75° C. 1 h, then the mixture was heated to 95° C. After 2 h, the reaction mixture was cooled to ambient temperature, azeotroped with toluene and crystallized from EtOAc/hexane (1:5) to afford 662 mg (32%) of di-tert-butyl 4-(2-chlorophenyl)-1,4-dihydro-2,6-dimethylpyridine-3,5-dicarboxylate as a white solid: MP 194-196° C.; ¹H NMR (500 MHz, CDCl₃) δ 1.38 (s, 18H), 2.21 (s, 6H), 5.34 (s, 1H), 5.56 (brs, 1H), 7.03-7.07 (m, 1H), 7.09-7.13 (m, 1H), 7.23-7.25 (m, 1H), 7.34-7.36 (m, 1H); ¹³C NMR (125 MHz, CDCl₃) δ 19.2, 28.3, 39.6, 79.9, 104.0, 126.0, 127.3, 129.7, 132.5, 132.8, 142.3, 143.9, 167.3; MS (ES) m/z 861 (2M+Na)⁺, 420 (M+H)⁺, 364, 290, 196; m/z 420.176 (calcd for C₂₃H₃₁ClNO₄ (M+H)⁺: 420.194).

### EXAMPLE 10

### Bis(2-methoxvethyl) 4-(2-chlorophenyl)-1,4-dihydro-2,6-dimethylpyridine-3,5-dicarboxylate

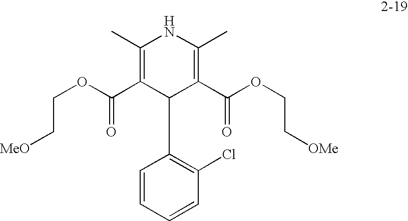

2-Methoxyethyl acetoacetate (1.51 mL, 97%, 10.0 mmol) and 2-chlorobenzaldehyde (562 μL, 99%, 5.00 mmol) were taken up in EtOH (1 mL) at rt. NH₄OH (500 μL) was added, the mixture was stirred at rt 1 h, then the mixture was heated to 95° C. After 2 h, the reaction mixture was cooled to ambient temperature, azeotroped with toluene and crystallized from EtOAc/hexane (1:5) to afford 1.04 g (49%) of dimethyl bis(2-methoxyethyl) 4-(2-chlorophenyl)-1,4-dihydro-2,6-dimethylpyridine-3,5-dicarboxylate as a white solid: MP 120-121° C.; ¹H NMR (500 MHz, CDCl₃) δ 2.29 (s, 6H), 3.32 (s, 6H), 3.58-3.72 (m, 4H), 4.11-4.24 (m, 4H), 5.43 (s, 1H), 5.96 (brs, 1H), 7.02-7.07 (m, 1H), 7.11-7.16 (m, 1H), 7.22-7.26 (m, 1H), 7.38-7.42 (m, 1H); ¹³C NMR (125 MHz, CDCl₃) δ 19.4, 37.7, 58.7, 62.5, 70.4, 103.3, 126.7, 127.3, 129.3, 131.8, 132.4, 144.4, 145.3, 167.5; MS (ES) m/z 847 (2M+H)⁺, 424 (M+H)⁺, 348; m/z 424.122 (calcd for C₂₁H₂₇ClNO₆ (M+H)⁺: 424.152).

### EXAMPLE 11

### Diethyl 4-(2-bromophenyl)-1,4-dihydro-2,6-dimethylpyridine-3,5-dicarboxylate

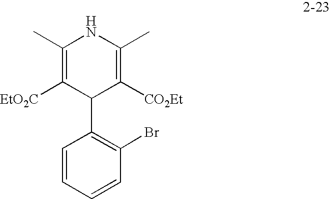

Ethyl acetoacetate (1.28 mL, 99%, 10.0 mmol) and 2-bromobenzaldehyde (604 μL, 97%, 5.00 mmol) were taken up in EtOH (1 mL) at rt. NH₄OH (500 μL) was added, then the mixture was heated to 95° C. After 2 h, the reaction mixture was cooled to ambient temperature, azeotroped with toluene and crystallized from EtOAc/hexane (1:9) to afford 312 mg (15%) of diethyl 4-(2-bromophenyl)-1,4-dihydro-2,6-dimethylpyridine-3,5-dicarboxylate as a white solid: MP 144-145° C.; ¹H NMR (500 MHz, CDCl₃) δ 1.20 (t, J=7.0 Hz, 6H), 2.30 (s, 6H), 4.10 (t, J=7.0 Hz, 2H), 4.11 (t, J=7.0 Hz, 2H), 5.36 (s, 1H), 5.61 (brs, 1H), 6.93-6.97 (m, 1H), 7.14-7.19 (m, 1H), 7.37-7.40 (m, 1H), 7.41-7.44 (m, 1H); ¹³C NMR (125 MHz, CDCl₃) δ 14.4, 19.6, 39.8, 59.7, 104.3, 122.7, 127.4, 127.6, 131.7, 132.7, 143.5, 147.4, 167.6; MS (ES) m/z 839 (2M+2H+Na)⁺, 408 (M+H)⁺, 364, 336, 282, 252; m/z 408.069 (calcd for C₁₉H₂₃BrNO₄ (M+H)⁺: 408.081).

### EXAMPLE 12

### Diethyl 4-(2-fluorophenyl)-1,4-dihydro-2,6-dimethylpyridine-3,5-dicarboxylate

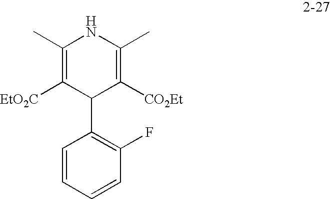

Ethyl acetoacetate (1.28 mL, 99%, 10.0 mmol) and 2-fluorobenzaldehyde (547 μL, 97%, 5.00 mmol) were taken up in EtOH (1 mL) at rt. NH₄OH (500 μL) was added, then the mixture was heated to 95° C. After 2 h, the reaction mixture was cooled to ambient temperature, azeotroped with toluene and crystallized from EtOAc/hexane (1:9) to afford 1.05 g (61%) of diethyl 4-(2-fluorophenyl)-1,4-dihydro-2,6-dimethylpyridine-3,5-dicarboxylate as a white solid: MP 151-152.5° C.; ¹H NMR (500 MHz, CDCl₃) δ 1.19 (t, J=7.2 Hz, 6H), 2.31 (s, 6H), 3.99-4.11 (m, 4H), 5.24 (s, 1H), 5.71 (brs, 1H), 6.87-6.92 (m, 1H), 6.96-7.01 (m, 1H), 7.06-7.12 (m, 1H), 7.28-7.32 (m, 1H); ¹³C NMR (125 MHz, CDCl₃) δ 14.0, 19.4, 34.2, 59.7, 103.0, 114.8, 115.0, 123.6, 127.6, 127.7, 131.1, 134.9, 135.0, 144.2, 158.8, 160.8, 167.5; MS (ES) m/z 717 (2M+Na)⁺, 370 (M+Na)⁺, 348 (M+H)⁺, 303, 274, 252; m/z 348.136 (calcd for C₁₉H₂₃FNO₄ (M+H)⁺: 348.161).

### EXAMPLE 13

### Di-tert-butyl 4-(2-fluorophenyl)-1,4-dihydro-2,6-dimethylpyridine-3,5-dicarboxylate

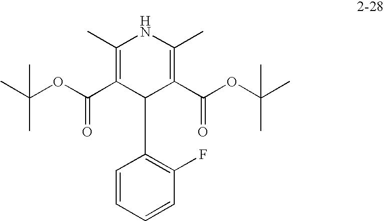

tert-Butyl acetoacetate (1.65 mL, 99%, 10.0 mmol) and 2-fluorobenzaldehyde (547 μL, 97%, 5.00 mmol) were taken up in EtOH (1 mL) at rt. NH₄OH (500 μL) was added, the reaction was stirred 1 h, then the mixture was heated to 95° C. After 2 h, the reaction mixture was cooled to ambient temperature, azeotroped with toluene and crystallized from EtOAc/hexane (1:9) to afford 313 mg (16%) of di-tert-butyl 4-(2-fluorophenyl)-1,4-dihydro-2,6-dimethylpyridine-3,5-dicarboxylate as a white solid: MP 201-202° C.; ¹H NMR (500 MHz, CDCl₃) δ 1.38 (s, 18H), 2.27 (s, 6H), 5.18 (s, 1H), 5.46 (brs, 1H), 6.87-6.92 (m, 1H), 6.96-7.01 (m, 1H), 7.06-7.11 (m, 1H), 7.26-7.31 (m, 1H); ¹³C NMR (125 MHz, CDCl₃) δ 19.4, 28.2, 34.9, 79.7, 104.2, 114.9, 115.0, 123.5, 127.5, 127.6, 131.3, 134.7, 143.0, 167.0; MS (ES) m/z 829 (2M+Na)⁺, 404 (M+H)⁺, 348, 274, 196; m/z 404.190 (calcd for C₂₃H₃₁FNO₄ (M+H)⁺: 404.223).

### EXAMPLE 14

### Diethyl 1,4-dihydro-2,6-dimethyl-4-(2-nitrophenyl)pyridine-3,5-dicarboxylate

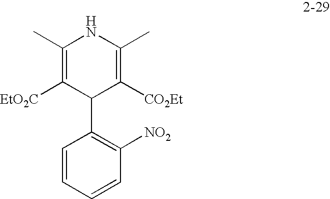

Ethyl acetoacetate (1.28 mL, 99%, 10.0 mmol) and 2-nitrobenzaldehyde (759 mg, 99+%, 5.00 mmol) were taken up in EtOH (1 mL) at rt. NH₄OH (500 μL) was added, the mixture was stirred at rt 1 h, 75° C. 1 h, then the mixture was heated to 95° C. After 2 h, the reaction mixture was cooled to ambient temperature, azeotroped with toluene and concentrated under reduced pressure. The residue was purified on a column of silica gel (0-10% MeOH/CH₂Cl₂) and crystallized from EtOAc/hexane (1:9) to afford 316 mg (17%) of diethyl 1,4-dihydro-2,6-dimethyl-4-(2-nitrophenyl)pyridine-3,5-dicarboxylate as a pale yellow solid: MP 120-121° C.; ¹H NMR (500 MHz, CDCl₃) δ 1.16 (t, J=7.0 Hz, 6H), 2.32 (s, 6H), 3.96-4.04 (m, 2H), 4.09-4.16 (m, 2H), 5.75 (brs, 1H), 5.85 (s, 1H), 7.23-7.28 (m, 1H), 7.44-7.48 (m, 1H), 7.52-7.55 (m, 1H), 7.72-7.75 (m, 1H); ¹³C NMR (125 MHz, CDCl₃) δ 14.1, 19.6, 34.6, 60.0, 103.9, 124.0, 126.9, 131.3, 132.7, 142.6, 144.5, 147.8, 167.2; MS (ES) m/z 787 (2M+K)⁺, 397 (M+Na)+, 375 (M+H)⁺, 357, 329, 285, 263; m/z 397.099 (calcd for C₁₉H₂₂N₂NaO₆ (M+Na)⁺: 397.138).

### EXAMPLE 14

### Di-tert-butyl 4-(2-bromophenyl)-1,4-dihydro-2,6-dimethylpyridine-3,5-dicarboxylate

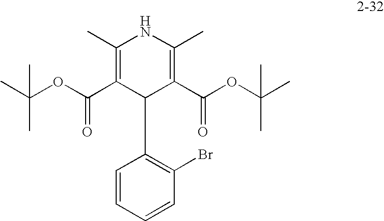

tert-Butyl acetoacetate (1.65 mL, 99%, 10.0 mmol) and 2-bromobenzaldehyde (604 μL, 97%, 5.00 mmol) were taken up in EtOH (1 mL) at rt. NH₄OH (500 μL) was added, the mixture was stirred at rt 1 h, then the mixture was heated to 95° C. After 2 h, the reaction mixture was cooled to ambient temperature, dried over Na₂SO₄, filtered and crystallized from EtOAc/hexane (1:9) to afford 654 mg (28%) of di-tert-butyl 4-(2-bromophenyl)-1,4-dihydro-2,6-dimethylpyridine-3,5-dicarboxylate as a white solid: MP 162-164° C.; ¹H NMR (500 MHz, CDCl₃) δ 1.37 (s, 18H), 2.19 (s, 6H), 5.33 (s, 1H), 5.50 (brs, 1H), 6.95-6.99 (m, 1H), 7.13-7.17 (m, 1H), 7.34-7.37 (m, 1H), 7.44-7.46 (m, 1H); ¹³C NMR (125 MHz, CDCl₃) δ 19.3, 28.3, 41.8, 79.9, 104.1, 122.6, 126.5, 127.5, 133.1, 132.2, 141.9, 145.3, 167.3; MS (ES) m/z 951 (2M+2H+Na)⁺, 464 (M+H)⁺, 408, 334, 196; m/z 464.129 (calcd for C₂₃H₃₁BrNO₄ (M+H)⁺: 464.163).

### EXAMPLE 15

### Di-tert-butyl 1,4-dihydro-2,6-dimethyl-4-(2-nitrophenyl)pyridine-3,5-dicarboxylate

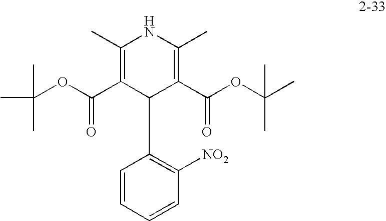

tert-Butyl acetoacetate (1.65 mL, 99%, 10.0 mmol) and 2-nitrobenzaldehyde (759 mg, 99+%, 5.00 mmol) were taken up in EtOH (1 mL) at rt. NH₄OH (500 μL) was added, the mixture was stirred at rt 1 h, 80° C. 1 h, then the mixture was heated to 95° C. After 2 h, the reaction mixture was cooled to ambient temperature, dried over Na₂SO₄, filtered and crystallized from EtOAc/hexane (1:9) to afford 200 mg (9%) of di-tert-butyl 1,4-dihydro-2,6-dimethyl-4-(2-nitrophenyl)pyridine-3,5-dicarboxylate as a pale yellow solid: MP 159-161° C.; ¹H NMR (500 MHz, CDCl₃) δ 1.36 (s, 18H), 2.22 (s, 6H), 5.63 (brs, 1H), 5.77 (s, 1H), 7.22-7.26 (m, 1H), 7.42-7.46 (m, 1H), 7.52-7.55 (m, 1H), 7.65-7.68 (m, 1H); ¹³C NMR 9125 MHz, CDCl₃) δ 19.5, 27.3, 28.1, 36.1, 80.3, 104.7, 123.9, 126.7, 131.7, 132.2, 141.9, 142.5, 148.3, 166.9; MS (ES) m/z 453 (M+Na)⁺, 431 (M+H)⁺, 413, 397, 357, 319, 301, 257, 239, 227; m/z 431.220 (calcd for C₂₃H₃₁N₂O₆ (M+H)⁺: 431.218).

### EXAMPLE 16

### Diallyl 4-(2-chlorophenyl)-1,4-dihydro-2,6-dimethylpyridine-3,5-dicarboxylate

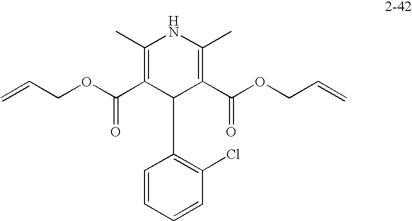

Allyl acetoacetate (1.40 mL, 98%, 10.0 mmol) and 2-chlorobenzaldehyde (562 μL, 99%, 5.00 mmol) were taken up in EtOH (1 mL) at rt. NH₄OH (500 μL) was added, the mixture was stirred at rt 1 h, 80° C. 1 h, then the mixture was heated to 95° C. After 2 h, the reaction mixture was cooled to ambient temperature, dried over Na₂SO₄, filtered and concentrated. The residue was purified on a column of silica gel (0-10% MeOH/CH₂Cl₂) and crystallized from EtOAc/hexane (1:20) to afford 392 mg (20%) of diallyl 4-(2-chlorophenyl)-1,4-dihydro-2,6-dimethylpyridine-3,5-dicarboxylate as a white solid: MP 98-99° C.; ¹H NMR (500 MHz, CDCl₃) δ 2.30 (s, 6H), 4.50-4.58 (m, 4H), 5.07-5.10 (m, 2H), 5.10-5.13 (m, 2H) 5.44 (s, 1H), 5.76 (brs, 1H), 5.81-5.90 (m, 2H), 7.01-7.06 (m, 1H), 7.09-7.14 9m, 1H), 7.20-7.23 (m, 1H), 7.36-7.39 (m, 1H); ¹³C NMR (125 MHz, CDCl₃) δ 19.6, 37.6, 64.5, 103.6, 117.3, 126.7, 127.3, 129.4, 131.6, 132.6, 132.9, 144.2, 145.4, 167.2; MS (ES) m/z 410 (M+Na)⁺, 388 (M+H)⁺, 330, 276; m/z 388.104 (calcd for C₂₃H₂₃ClNO₄ (M+H)⁺: 388.131).

### EXAMPLE 17

### Dimethyl 4-(2-chlorophenyl)-1,4-dihydro-2,6-bis(methoxymethyl)pyridine-3,5-dicarboxylate

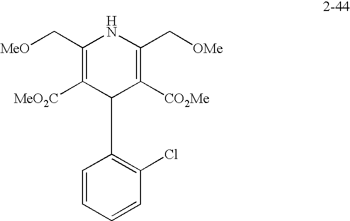

Methyl 4-methoxyacetoacetate (1.33 mL, 97%, 10.0 mmol) and 2-chlorobenzaldehyde (562 μL, 99%, 5.00 mmol) were taken up in EtOH (1 mL) at rt. NH₄OH (500 μL) was added, the mixture was stirred at rt 1 h, 80° C. 1 h, then the mixture was heated to 95° C. After 2 h, the reaction mixture was cooled to ambient temperature, dried over Na₂SO₄, filtered and crystallized from EtOAc/hexane (1:9) to afford 54 mg (3%) of dirnethyl 4-(2-chlorophenyl)-1,4-dihydro-2,6-bis(methoxymethyl)pyridine-3,5-dicarboxylate as a white solid: MP 137-138° C.; ¹H NMR (500 MHz, CDCl₃) δ 3.48 (s, 6H), 3.61 (s, 6H), 4.64 (d, J=16.0 Hz, 2H), 4.73 (d, J=16.2 Hz, 2H), 5.10-5.13 (m, 2H) 5.45 (s, 1H), 7.03-7.07 (m, 1H), 7.12-7.17 (m, 1H), 7.23-7.26 (m, 1H), 7.37-7.40 (m, 1H), 8.36 (brs, 1H); ¹³C NMR (125 MHz, CDCl₃) δ 36.8, 50.7, 69.0, 69.7, 101.5, 127.0, 127.4, 129.2, 131.3, 132.2, 145.3, 145.7, 167.4; MS (ES) m/z 813 (2M+Na)⁺, 418 (M+Na)⁺, 396 (M+H)⁺, 364, 332, 284; m/z 396.098 (calcd for C₁₉H₂₃ClNO₆ (M+H)⁺: 396.121).

### EXAMPLE 18

### Diethyl 1,4-dihydro-4-(2-iodophenyl)-2,6-dimethylpyridine-3,5-dicarboxylate

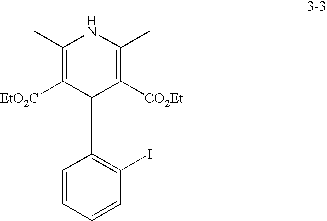

Ethyl acetoacetate (511 μL, 99%, 4.00 mmol) and 2-iodobenzaldehyde (478 mg, 97%, 2.00 mmol) were taken up in EtOH (400 μL) at rt. NH₄OH (200 μL) was added, the mixture was stirred at rt 1 h, then the mixture was heated to 95° C. After 2 h, the reaction mixture was cooled to ambient temperature, diluted with CH₂Cl₂, dried over Na₂SO₄ and filtered. Crystallization from CH₂Cl₂/hexanes (1:9) afforded 495 mg (54%) of diethyl 1,4-dihydro-4-(2-iodophenyl)-2,6-dimethylpyridine-3,5-dicarboxylate as a white solid: MP 173-174.5° C.; ¹H NMR (500 MHz, CDCl₃) δ 1.22 (t, J=7.1 Hz, 6H), 2.30 (s, 6H), 4.12-4.22 (m, 4H), 5.18 (s, 1H), 5.66 (brs, 1H), 6.79 (t, J=7.6 Hz, 1H), 7.22 (t, J=7.6 Hz, 1H), 7.38 (d, J=7.8 Hz, 1H), 7.75 (d, J=7.8 Hz, 1H); ¹³C NMR (125 MHz, CDCl₃) δ 14.6, 19.6, 43.8, 59.7, 98.6, 104.7, 127.7, 128.4, 130.9, 139.6, 143.2, 150.8, 167.6; MS (ES) m/z 933 (2M+Na)⁺, 478 (M+Na)⁺, 456 (M+H)⁺, 410, 283, 254, 210; m/z 456.056 (calcd for C₁₉H₂₃INO₄ (M+H)⁺: 456.067).

### EXAMPLE 19

### Dimethyl 4-(2-bromophenyl)-1,4-dihydro-2,6-dimethylpyridine-3,5-dicarboxylate

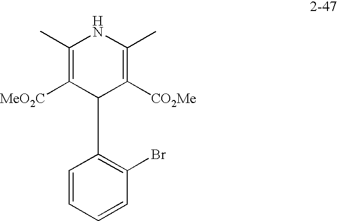

Methyl acetoacetate (545 μL, 99+%, 5.00 inmol), 2-bromobenzaldehyde (604 μL, 97%, 5.00 mmol) and methyl-3-aminocrotonate (593 mg, 97%, 5.00 mmol) were taken up in EtOH (3.25 mL) at rt. AcOH (217 μL) was added and the mixture was heated to 95° C. After 3 h, the reaction mixture was cooled to ambient temperature, diluted with EtOAc (20 mL), dried over Na₂SO₄, filtered and concentrated. Crystallization from EtOAc/hexanes (1:9) afforded 384 mg (20%) of dimethyl 4-(2-bromophenyl)-1,4-dihydro-2,6-dimethylpyridine-3,5-dicarboxylate as a white solid: MP 164-165° C.; ¹H NMR (500 MHz, CDCl₃) δ 2.32 (s, 6H), 3.63 (s, 6H), 5.36 (s, 1H), 5.62 (brs, 1H), 7.02-7.07 (m, 1H), 7.15-7.19 (m, 1H), 7.36-7.39 (m, 1H), 7.41-7.44 (m, 1H); ¹³C NMR (125 MHz, CDCl₃) δ 19.5, 39.3, 50.8, 104.3, 122.6, 127.6, 127.7, 131.2, 132.6, 143.9, 147.8, 168.0; MS (ES) m/z 783 (2M+2H+Na)⁺, 402 (M+Na)⁺, 380 (M+H)⁺, 348, 268, 224; m/z 380.032 (calcd for C₁₇H₁₉BrNO₄ (M+H)⁺: 380.049).

### EXAMPLE 20

### Diethyl 4-(3-chlorophenyl)-1,4-dihydro-2,6-dimethylpyridine-3,5-dicarboxylate

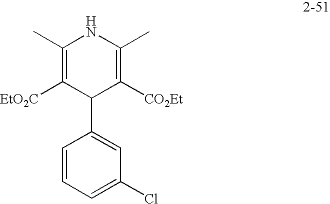

Ethyl acetoacetate (1.28 mL, 99%, 10.0 mmol) and 2-chlorobenzaldehyde (572 μL, 99%, 5.00 mmol) were taken up in EtOH (1 mL) at rt. NH₄OH (500 μL), the mixture was stirred at ambient temperature for 1 h, then heated to 95 ° C. After 3 h, the reaction mixture was cooled to ambient temperature, diluted with CH₂Cl₂ (10 mL), dried over Na₂SO₄, filtered hexanes (90 mL) were added. Crystallization afforded 967 mg (53%) of diethyl 4-(3-chlorophenyl)-1,4-dihydro-2,6-dimethylpyridine-3,5-dicarboxylate as a white solid: MP 142.5-143.5° C.; ¹H NMR (500 MHz, CDCl₃) δ 1.25 (t, J=7.1 Hz, 6H), 2.36 (s, 6H), 4.05-4.18 (m, 4H), 4.99 (s, 1H), 5.63 (brs, 1H), 7.10-7.20 (m, 3H), 7.26 (t, J=1.7 Hz, 1H); ¹³C NMR (125 MHz, CDCl₃) δ 14.2, 19.6, 39.7, 59.8, 103.7, 126.2, 126.3, 128.3, 129.0, 133.6, 144.1, 149.7, 167.3; MS (ES) m/z 749 (2M+2H+Na)⁺, 386 (M+Na)⁺, 364 (M+H)⁺, 318, 272, 252; m/z 364.104 (calcd for C₁₉H₂₃ClNO₄ (M+H)⁺: 364.131).

### EXAMPLE 21

### Di-tert-butyl 4-(3-chlorophenyl)-1,4-dihydro-2,6-dimethylpyridine-3,5-dicarboxylate

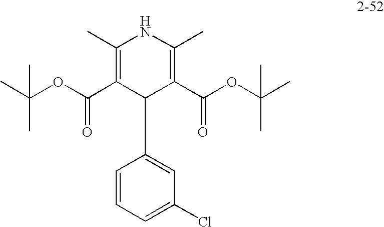

tert-Butyl acetoacetate (1.65 mL, 99%, 10.0 mmol) and 3-chlorobenzaldehyde (572 μL, 99%, 5.00 mmol) were taken up in EtOH (1 mL) at rt. NH₄OH (500 μL) was added, the mixture was stirred at rt 1 h, then the mixture was heated to 95° C. After 3 h, the reaction mixture was cooled to ambient temperature, diluted with CH₂Cl₂ (10 mL) and dried over Na₂SO₄. Crystallization from CH₂Cl₂/hexane (1:9) afforded 730 mg (35%) of di-tert-butyl 4-(3-chlorophenyl)-1,4-dihydro-2,6-dimethylpyridine-3,5-dicarboxylate as a white solid: MP 189.5-190.5° C.; ¹H NMR (500 MHz, CDCl₃) δ 1.42 (s, 18H), 2.31 (s, 6H), 4.90 (s, 1H), 5.51 (brs, 1H), 7.09-7.20 (m, 3H), 7.25-7.27 (m, 1H); ¹³C NMR (125 MHz, CDCl₃) δ 19.5, 28.3, 40.3, 79.8, 104.9, 126.0, 126.2, 128.3, 128.9, 133.4, 143.1, 149.9, 166.8; MS (ES) m/z 861 (2M+Na)⁺, 442 (M+Na)⁺, 386, 290, 196; m/z 442.158 (calcd for C₂₃H₃₀NNaO₄ (M+Na)⁺: 442.176).

### EXAMPLE 22

### Diethyl 4-(4-chlorophenyl)-1,4-dihydro-2,6-dimethylpyridine-3,5-dicarboxylate

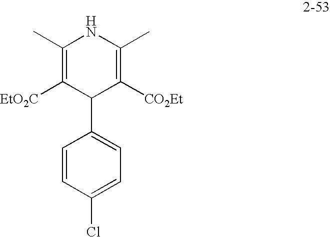

Ethyl acetoacetate (1.28 mL, 99%, 10.0 mmol) and 4-chlorobenzaldehyde (714 mg, 98.5%, 5.00 mmol) were taken up in EtOH (1 mL) at rt. NH₄OH (500 μL), the mixture was stirred at ambient temperature for 1 h, then heated to 95° C. After 3 h, the reaction mixture was cooled to ambient temperature, diluted with CH₂Cl₂ (10 mL) and dried over Na₂SO₄. Crystallization from CH₂Cl₂/hexane (1:9) afforded 1.24 g (68%) of diethyl 4-(4-chlorophenyl)-1,4-dihydro-2,6-dimethylpyridine-3,5-dicarboxylate as a white solid: MP 151-152° C.; ¹H NMR (500 MHz, CDCl₃) δ 1.23 (t, J=7.1 Hz, 6H), 2.34 (s, 6H), 4.05-4.16 (m, 4H), 4.98 (s, 1H), 5.68 (brs, 1H), 7.16-7.20 (m, 2H), 7.21-7.24 (m, 2H); ¹³C NMR (125 MHz, CDCl₃) δ 14.2, 19.6, 39.2, 59.8, 103.9, 127.9, 129.4, 131.7, 143.9, 146.3, 167.3; MS (ES) m/z 749 (2M+Na)⁺, 386 (M+Na)⁺, 364 (M+H)⁺, 319, 290, 252; m/z 364.112 (calcd for C₁₉H₂₃ClNO₄ (M+H)⁺: 364.131).

### EXAMPLE 23

### Di-tert-butyl 4-(4-chlorophenyl)-1,4-dihydro-2,6-dimethylpyridine-3,5-dicarboxylate

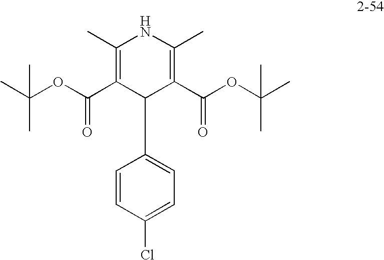

tert-Butyl acetoacetate (1.65 mL, 99%, 10.0 mmol) and 4-chlorobenzaldehyde (714 mg, 98.5%, 5.00 mmol) were taken up in EtOH (1 mL) at rt. NH₄OH (500 μL) was added, the mixture was stirred at rt 1 h, then the mixture was heated to 95° C. After 3 h, the reaction mixture was cooled to ambient temperature, diluted with CH₂Cl₂ (10 mL) and dried over Na₂SO₄. The crude product was crystallized from CH₂Cl₂/hexane (1:9) to afford 956 mg (46%) of di-tert-butyl 4-(4-chlorophenyl)-1,4-dihydro-2,6-dimethylpyridine-3,5-dicarboxylate as a white solid: MP 191.5-192.5° C.; ¹H NMR (500 MHz, CDCl₃) δ 1.41 (s, 18H), 2.30 (s, 6H), 4.90 (s, 1H), 5.45 (brs, 1H), 7.17-7.24 (m, 4H); ¹³C NMR (125 MHz, CDCl₃) δ 24.9, 33.6, 45.1, 85.1, 110.4, 133.1, 134.7, 136.8, 148.2, 151.8, 172.1; MS (ES) m/z 861 (2M+Na)⁺, 442 (M+Na)⁺, 386, 290, 224; m/z 442.141 (calcd for C₂₃H₃₀NNaO₄ (M+Na)⁺: 442.176).

### EXAMPLE 24

### Diethyl 4-(3-bromophenyl)-1,4-dihydro-2,6-dimethylpyridine-3,5-dicarboxylate

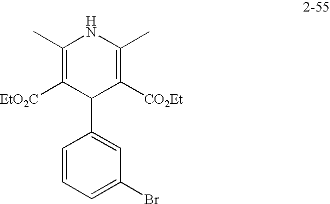

Ethyl acetoacetate (1.28 mL, 99%, 10.0 mmol) and 3-bromobenzaldehyde (964 mg, 96%, 5.00 mmol) were taken up in EtOH (1 mL) at rt. NH₄OH (500 μL), the mixture was stirred at ambient temperature for 1 h, then heated to 95° C. After 2 h, the reaction mixture was cooled to ambient temperature, diluted with CH₂Cl₂ (10 mL) and dried over Na₂SO₄. Crystallization from CH₂Cl₂/hexane (1:9) afforded 1.46 g (71%) of diethyl 4-(3-bromophenyl)-1,4-dihydro-2,6-dimethylpyridine-3,5-dicarboxylate as a white solid: MP 125-126° C.; ¹H NMR (500 MHz, CDCl₃) δ 1.24 (t, J=7.1 Hz, 6H), 2.35 (s, 6H), 4.04-4.16 (m, 4H), 4.99 (s, 1H), 5.68 (brs, 1H), 7.09 (t, J=7.8 Hz, 1H), 7.21-7.28 (m, 2H), 7.40-7.42 (m, 1H); ¹³C NMR (125 MHz, CDCl₃) δ 14.2, 19.6, 39.7, 59.8, 103.7, 121.9, 126.8, 129.2, 129.4, 131.2, 144.1, 150.0, 167.3; MS (ES) m/z 839 (2M+Na)⁺, 430 (M+Na)⁺, 408 (M+H)⁺, 364, 315, 252; m/z 408.061 (calcd for C₁₉H₂₃BrNO₄ (M+H)⁺: 408.081).

### EXAMPLE 25

### Di-tert-butyl 4-(3-bromophenyl)-1,4-dihydro-2,6-dimethylpyridine-3,5-dicarboxylate

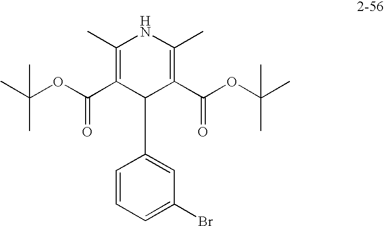

tert-Butyl acetoacetate (1.65 mL, 99%, 10.0 mmol) and 3-bromobenzaldehyde (964 mg, 96%, 5.00 mmnol) were taken up in EtOH (1 mL) at rt. NH₄OH (500 μL) was added, the mixture was stirred at rt 1 h, then the mixture was heated to 95° C. After 2 h, the reaction mixture was cooled to ambient temperature, diluted with CH₂Cl₂ (10 mL) and dried over Na₂SO₄. The crude product was crystallized from CH₂CI₂/hexane (1:9) to afford 1.11 g (48%) of di-tert-butyl 4-(3-bromophenyl)-1,4-dihydro-2,6-dimethylpyridine-3,5-dicarboxylate as a white solid: MP 195-196° C.; ¹H NMR (500 MHz, CDCl₃) δ 1.42 (s, 18H), 2.31 (s, 6H), 4.89 (s, 1H), 5.49 (brs, 1H), 7.09 (t, J=7.8 Hz, 4H), 7.20-7.28 (m, 2H), 7.41-7.43 (m, 1H); ¹³C NMR (125 MHz, CDCl₃) δ 19.5, 28.3, 40.3, 79.9, 104.9, 121.7, 126.7, 128.9, 129.3, 131.2, 143.1, 150.2, 166.8; MS (ES) m/z 951 (2M+Na)⁺, 486 (M+Na)⁺, 430, 334, 196; m/z 486.118 (calcd for C₂₃H₃₀BrNNaO₄ (M+Na)⁺: 486.126).

### EXAMPLE 26

### Diethyl 4-(4-bromophenyl)-1,4-dihydro-2,6-dimethylpyridine-3,5-dicarboxylate

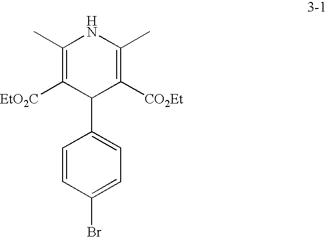

Ethyl acetoacetate (1.28 mL, 99%, 10.0 mmol) and 4-bromobenzaldehyde (934 mg, 99%, 5.00 mmnol) were taken up in EtOH (2 mL) at rt. NH₄OH (500 μL), the mixture was stirred at ambient temperature for 1 h, then heated to 95° C. After 2 h, the reaction mixture was cooled to ambient temperature, diluted with CH₂Cl₂ (10 mL) and dried over Na₂SO₄. Crystallization from CH₂Cl₂/hexane (1:9) afforded 1.35 g (66%) of diethyl 4-(4-bromophenyl)-1,4-dihydro-2,6-dimethylpyridine-3,5-dicarboxylate as a white solid: MP 164-165° C.; ¹H NMR (500 MHz, CDCl₃) δ 1.24 (t, J=7.1 Hz, 6H), 2.34 (s, 6H), 4.05-4.16 (m, 4H), 4.96 (s, 1H), 5.64 (brs, 1H), 7.15-7.19 (m, 2H), 7.32-7.36 (m, 2H); ¹³C NMR (125 MHz, CDCl₃) δ 14.2, 19.6, 39.3, 59.8, 103.8, 119.8, 129.8, 130.9, 143.9, 146.8, 167.3; MS (ES) m/z 839 (2M+Na)⁺, 430 (M+Na)⁺, 408 (M+H)⁺, 364, 334, 252; m/z 408.061 (calcd for C₁₉H₂₃BrNO₄ (M+H)⁺: 408.081).

### EXAMPLE 27

### Di-tert-butyl 4-(4-bromophenyl)-1,4-dihydro-2,6-dimethylpyridine-3,5-dicarboxylate

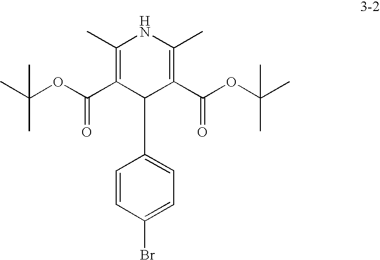

tert-Butyl acetoacetate (1.65 mL, 99%, 10.0 mmol) and 3-bromobenzaldehyde (934 mg, 99%, 5.00 mmol) were taken up in EtOH (2 mL) at rt. NHIOH (500 μL) was added, the mixture was stirred at rt 1 h, then the mixture was heated to 95° C. After 2 h, the reaction mixture was cooled to ambient temperature, diluted with CH₂Cl₂ (10 mL) and dried over Na₂SO₄. The crude product was crystallized from CH₂Cl₂/hexane (1:9) to afford 863 mg (37%) of di-tert-butyl 4-(4-bromophenyl)-1,4-dihydro-2,6-dimethylpyridine-3,5-dicarboxylate as a white solid: MP 206-207° C.; ¹H NMR (500 MHz, CDCl₃) δ 1.41 (s, 18H), 2.30 (s, 6H), 4.89 (s, 1H), 5.49 (brs, 1H), 7.15-7.18 (m, 2H), 7.32-7.36 (m, 2H); ¹³C NMR (125 MHz, CDCl₃) δ 19.5, 28.3, 39.8, 79.8, 105.0, 119.6, 129.8, 130.7, 142.9, 147.0, 166.8; MS (ES) m/z 486 (M+Na)⁺, 464 (M+H)⁺, 352, 334, 196; m/z 464.137 (calcd for C₂₃H₃₁BrNO₄ (M+H)⁺: 464.143).

### EXAMPLE 28

### Di-tert-butyl 1,4-dihydro-4-(2-iodophenyl)-2,6-dimethylpyridine-3,5-dicarboxylate

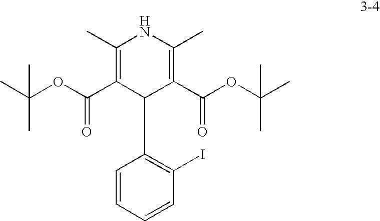

tert-Butyl acetoacetate (659 μL, 99%, 4.00 mmol) and 2-iodobenzaldehyde (478 mg, 99%, 2.00 mmol) were taken up in EtOH (400 μL) at rt. NH₄OH (200 μL) was added, the mixture was stirred at rt 1 h, then the mixture was heated to 95° C. After 2 h, the reaction mixture was cooled to ambient temperature, diluted with CH₂Cl₂ (10 mL) and dried over Na₂SO₄. The crude product purified on a column of silica gel (0-10% MeOH/CH₂Cl₂ as eluent) and crystallized from CH₂Cl₂/hexane (1:9) to afford 46 mg (4%) of di-tert-butyl 1,4-dihydro-4-(2-iodophenyl)-2,6-dimethylpyridine-3,5-dicarboxylate as a white solid: MP 184-185° C.; ¹H NMR (500 MHz, CDCl₃) δ 1.37 (s, 18H), 2.18 (s, 6H), 5.25 (s, 1H), 5.42 (brs, 1H), 6.79-6.84 (m, 1H), 7.19-7.24 (m, 1H), 7.33-7.36 (m, 1H), 7.79-7.82 (m, 1H); ¹³C NMR (125 MHz, CDCl₃) δ 19.3, 28.3, 45.4, 80.0, 97.1, 104.2, 127.1, 127.7, 133.4, 140.5, 141.5, 147.7, 167.2; MS (ES) m/z 1045 (2M+Na)⁺, 534 (M+Na)⁺, 512 (M+H)⁺, 478, 382, 294, 255; m/z 512.115 (calcd for C₂₃H₃₁INO₄ (M+H)⁺: 512.129).

### EXAMPLE 29

### Diethyl 1,4-dihydro-2,6-dimethyl-4-phenylpuridine-3,5-dicarboxylate

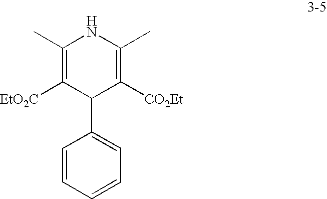

Ethyl acetoacetate (1.28 mL, 99%, 10.0 mmol) and benzaldehyde (508 μL, 99.5%, 5.00 mmol) were taken up in EtOH (1 mL) at rt. NH₄OH (500 μL), the mixture was stirred at ambient temperature for 1 h, then heated to 95° C. After 2 h, the reaction mixture was cooled to ambient temperature, diluted with CH₂Cl₂ (10 mL) and dried over Na₂SO₄. Crystallization from CH₂Cl₂/hexane (1:9) afforded 1.02 g (62%) of diethyl 1,4-dihydro-2,6-dimethyl-4-phenylpyridine-3,5-dicarboxylate as a white solid: MP 158-159° C.; ¹H NMR (500 MHz, CDCl₃) δ 1.24 (t, J=7.1 Hz, 6H), 2.33 (s, 6H), 4.05-4.16 (m, 4H), 5.01 (s, 1H), 5.86 (brs, 1H), 7.11-7.16 (m, 1H), 7.20-7.24 (m, 2H), 7.27-7.32 (m, 2H); ¹³C NMR (125 MHz, CDCl₃) δ 14.2, 19.5, 39.6, 59.7, 104.0, 126.0, 127.8, 127.9, 143.9, 147.7, 167.6; MS (ES) m/z 681 (2M+Na)⁺, 352 (M+Na)⁺, 330 (M+H)⁺, 284, 256; m/z 330.152 (calcd for C₁₉H₂₄NO₄ (M+H)⁺: 330.170).

### EXAMPLE 30

### Di-tert-butyl 1,4-dihydro-2,6-dimethyl-4-phenylpyridine-3,5-dicarboxylate

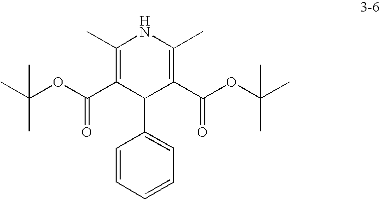

tert-Butyl acetoacetate (1.65 mL, 99%, 10.0 mmol) and 3-bromobenzaldehyde (508 μL, 99.5%, 5.00 mmol) were taken up in EtOH (1 mL) at rt. NH₄OH (500 μL) was added, the mixture was stirred at rt 1 h, then the mixture was heated to 95° C. After 2 h, the reaction mixture was cooled to ambient temperature, diluted with CH₂Cl₂ (10 mL) and dried over Na₂SO₄. The crude product was crystallized from CH₂Cl₂/hexane (1:9) to afford 448 mg (23%) of di-tert-butyl 1,4-dihydro-2,6-dimethyl-4-phenylpyridine-3,5-dicarboxylate as a white solid: MP 187-188° C.; ¹H NMR (500 MHz, CDCl₃) δ 1.41 (s, 18H), 2.29 (s, 6H), 4.93 (s, 1H), 5.59 (brs, 1H), 7.10-7.15 (m, 1H), 7.19-7.24 (m, 2H), 7.26-7.30 (m, 2H); ¹³C NMR (125 MHz, CDCl₃) δ 19.4, 28.2, 40.2, 79.6, 105.3, 125.8, 127.7, 127.9, 128.0, 142.8, 147.9, 167.1; MS (ES) m/z 793 (2M+Na)⁺, 408 (M+Na)⁺, 386 (M+H)⁺, 352, 256, 196; m/z 386.215 (calcd for C₂₃H₃₂NO₄ (M+H)⁺: 386.233).

### EXAMPLE 31

### Diethyl 4-(2,3-dichlorophenyl)-1,4-dihydro-2,6-dimethylpyridine-3,5-dicarboxylate

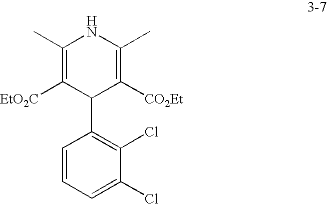

Ethyl acetoacetate (1.28 mL, 99%, 10.0 mmol) and 2,3-dichlorobenzaldehyde (854 mg, 99%, 5.00 mmol) were taken up in EtOH (2 mL) at rt. NH₄OH (500 μL), the mixture was stirred at ambient temperature for 1 h, then heated to 95° C. After 2 h, the reaction mixture was cooled to ambient temperature, diluted with CH₂Cl₂ (10 mL) and dried over Na₂SO₄. Purification on a column of silica gel (0-10% MeOH/CH₂Cl₂ as eluent) and crystallization from CH₂Cl₂/hexane (1:9) afforded 409 mg (21%) of diethyl 4-(2,3-dichlorophenyl)-1,4-dihydro-2,6-dimethylpyridine-3,5-dicarboxylate as a white solid: MP 125-126° C.; ¹H NMR (500 MHz, CDCl₃) δ 1.20 (t, J=7.1 Hz, 6H), 2.31 (s, 6H), 4.09 (q, J=7.21 Hz, 4H), 5.48 (s, 1H), 5.73 (brs, 1H), 7.08 (t, J=7.8 Hz, 1H), 7.26 (dd, J=1.5, 7.9 Hz, 1H), 7.32 (dd, J=1.5, 7.8 Hz, 1H); ¹³C NMR (125 MHz, CDCl₃) δ 14.3, 19.6, 38.8, 59.8, 103.6, 126.9, 128.2, 129.9, 131.0, 132.7, 144.0, 148.0, 167.4; MS (ES) m/z 819 (2M+Na)⁺, 420 (M+Na)⁺, 398 (M+H)⁺, 352, 324, 252; m/z 398.061 (calcd for C₁₉H₂₂Cl₂NO₄ (M+H)⁺: 398.092).

### EXAMPLE 32

### Di-tert-butyl 4-(2,3-dichlorophenyl)-1,4-dihydro-2,6-dimethylpyridine-3,5-dicarboxylate

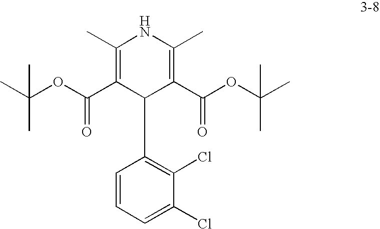

tert-Butyl acetoacetate (1.65 mL, 99%, 10.0 mmol) and 2,3-chlorobenzaldehyde (884 mg, 99%, 5.00 mmol) were taken up in EtOH (2 mL) at rt. NH₄OH (500 μL) was added, the mixture was stirred at rt 1 h, then the mixture was heated to 95° C. After 2 h, the reaction mixture was cooled to ambient temperature, diluted with CH₂Cl₂ (10 mL) and dried over Na₂SO₄. The crude product was crystallized from CH₂Cl₂/hexane (1:9) to afford 221 mg (10%) of di-tert-butyl 4-(2,3-dichlorophenyl)-1,4-dihydro-2,6-dimethylpyridine-3,5-dicarboxylate as a white solid: MP 144-145° C.; ¹H NMR (500 MHz, CDCl₃) δ 1.39 (s, 18H), 2.23 (s, 6H), 5.41 (s, 1H), 5.57 (brs, 1H), 7.08 (t, J=7.8 Hz, 2H), 7.26-7.33 (m, 2H); ¹³C NMR (125 MHz, CDCl₃) δ 19.4, 28.3, 40.7, 80.1, 103.7, 126.3, 128.2, 130.9, 131.4, 133.0, 142.6, 146.2, 167.0; MS (ES) m/z 454 (M+H)⁺, 398, 324, 196; m/z 454.104 (calcd for C₂₃H₃₀Cl₂NO₄ (M+H)⁺: 454.155).

### EXAMPLE 33

### Diethyl 4-(2,4-dichlorophenyl)-1,4-dihydro-2,6-dimethylpyridine-3,5-dicarboxylate

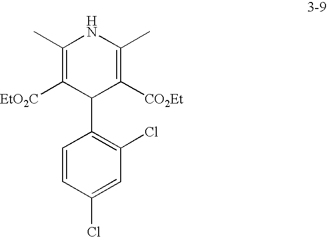

Ethyl acetoacetate (1.28 mL, 99%, 10.0 mmol) and 2,4-dichlorobenzaldehyde (893 mg, 98%, 5.00 mmol) were taken up in EtOH (2 mL) at rt. NH₄OH (500 μL), the mixture was stirred at ambient temperature for 1 h, then heated to 95° C. After 2 h, the reaction mixture was cooled to ambient temperature, diluted with CH₂Cl₂ (10 mL) and dried over Na₂SO₄. Crystallization from CH₂Cl₂/hexane (1:9) afforded 1.01 g (51%) of diethyl 4-(2,4-dichlorophenyl)-1,4-dihydro-2,6-dimethylpyridine-3,5-dicarboxylate as a white solid: MP 148-149° C.; ¹H NMR (500 MHz, CDCl₃) δ 1.21 (t, J=7.1 Hz, 6H), 2.30 (s, 6H), 4.04-4.14 (m, 4H), 5.36 (s, 1H), 5.89 (brs, 1H), 7.11 (dd, J=2.1, 8.4 Hz, 1H), 7.26 (d, J=2.1 Hz, 1H), 7.31 (t, J=7.0 Hz, 1H); ¹³C NMR (125 MHz, CDCl₃) δ 14.3, 19.5, 37.3, 59.8, 103.4, 127.0, 128.8, 132.1, 132.5, 133.1, 144.2, 144.3, 167.4; MS (ES) m/z 819 (2M+Na)⁺, 420 (M+Na)⁺, 398 (M+H)⁺, 352, 324, 252; m/z 398.077 (calcd for C₁₉H₂₂Cl₂NO₄ (M+H)⁺: 398.092).

### EXAMPLE 34

### Diethyl 4-(2,5-dichlorophenyl)-1,4-dihydro-2,6-dimethylpyridine-3,5-dicarboxylate

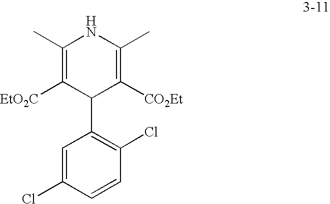

Ethyl acetoacetate (640 μL, 99%, 5.00 mmol) and 2,5-dichlorobenzaldehyde (446 mg, 98%, 2.50 mmol) were taken up in EtOH (500 μL) at rt. NH₄OH (250 μL), the mixture was stirred at ambient temperature for 1 h, then heated to 95° C. After 3 h, the reaction mixture was cooled to ambient temperature, diluted with CH₂Cl₂ (10 mL) and dried over Na₂SO₄. Crystallization from CH₂Cl₂/hexane (1:9) afforded 546 mg (55%) of diethyl 4-(2,5-dichlorophenyl)-1,4-dihydro-2,6-dimethylpyridine-3,5-dicarboxylate as a white solid: MP 166.5-167.5° C.; ¹H NMR (500 MHz, CDCl₃) δ 1.22 (t, J=7.1 Hz, 6H), 2.34 (s, 6H), 4.10 (q, J=7.1 Hz, 4H), 5.36 (s, 1H), 5.65 (brs, 1H), 7.04 (dd, J=2.6, 8.5 Hz, 1H), 7.18 (d, J=8.5 Hz, 1H), 7.33 (d, J=2.5 Hz, 1H); ¹³C NMR (125 MHz, CDCl₃) δ 14.3, 19.7, 38.1, 59.8, 103.3, 127.4, 130.4, 131.0, 131.6, 132.2, 144.2, 147.1, 167.3; MS (ES) m/z 819 (2M+Na)⁺, 420 (M+Na)⁺, 398 (M+H)⁺, 352, 324, 252; m/z 398.077 (calcd for C₁₉H₂₂Cl₂NO₄ (M+H)⁺: 398.092).

### EXAMPLE 35

### Di-tert-butyl 4-(2,5-dichlorophenyl)-1,4-dihydro-2,6-dimethylpyridine-3,5-dicarboxylate

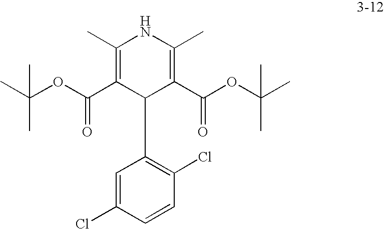

tert-Butyl acetoacetate (825 μL, 99%, 5.00 mmol) and 2,3-chlorobenzaldehyde (446 mg, 98%, 2.50 mmol) were taken up in EtOH (500 μL) at rt. NH₄OH (250 μL) was added, the mixture was stirred at rt 1 h, then the mixture was heated to 95° C. After 2 h, the reaction mixture was cooled to ambient temperature, diluted with CH₂Cl₂ (10 mL) and dried over Na₂SO₄. The crude product was crystallized from CH₂Cl₂/hexane (1:9) to afford 134 mg (12%) of di-tert-butyl 4-(2,5-dichlorophenyl)-1,4-dihydro-2,6-dimethylpyridine-3,5-dicarboxylate as a white solid: MP 181-183° C.; ¹H NMR (500 MHz, CDCl₃) δ 1.40 (s, 18H), 2.24 (s, 6H), 5.27 (s, 1H), 5.59 (brs, 1H), 7.06 (dd, J=2.4, 8.5 Hz, 1H), 7.20 (d, J=8.5 Hz, 1H), 7.32 (d, J=2.4 Hz, 1H); ¹³C NMR (125 MHz, CDCl₃) δ 19.4, 28.3, 40.4, 80.0, 103.1, 127.3, 131.0, 131.5, 131.6, 132.8, 143.1, 145.2, 166.9; MS (ES) m/z 476 (M+Na)⁺, 454 (M+H)⁺, 420, 196; m/z 454.147 (calcd for C₂₃H₃₀Cl₂NO₄ (M+H)⁺: 454.155).

### EXAMPLE 36

### Diethyl 4-(2,6-dichlorophenyl)-1,4-dihydro-2,6-dimethylpyridine-3,5-dicarboxylate

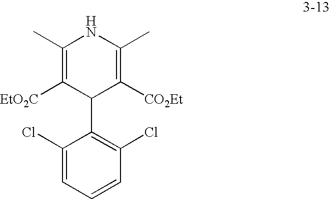

Ethyl acetoacetate (1.28 mL, 99%, 10.0 mmol) and 2,6-dichlorobenzaldehyde (884 mg, 99%, 5.00 mmol) were taken up in EtOH (1 mL) at rt. NH₄OH (500 μL), the mixture was stirred at ambient temperature for 1 h, then heated to 95° C. After 2 h, the reaction mixture was cooled to ambient temperature, diluted with CH₂Cl₂ (10 mL) and dried over Na₂SO₄. Purification on a column of silica gel (0-10% MeOH/CH₂Cl₂ as eluent) and crystallization from CH₂Cl₂/hexane (1:9) afforded 89 mg (4%) of diethyl 4-(2,6-dichlorophenyl)-1,4-dihydro-2,6-dimethylpyridine-3,5-dicarboxylate as a white solid: MP 134-135° C.; ¹H NMR (500 MHz, CDCl₃) δ 1.09-1.14 (m, 6H), 2.23-2.25 (m, 6H), 4.02-4.08 (m, 4H), 5.73 (s, 1H), 5.92 (brs, 1H), 6.97-7.03 (m, 1H), 7.23-7.26 (m, 2H); ¹³C NMR (125 MHz, CDCl₃) δ 14.2, 19.7, 37.8, 59.5, 100.4, 127.2, 137.2, 139.9, 145.0, 167.6; MS (ES) m/z 819 (2M+Na)⁺, 396 (M−H)⁺, 352, 252; m/z 396.774 (calcd for C₁₉H₂₀Cl₂NO₄ (M−H): 396.077).

### EXAMPLE 37

### Diethyl 4-(3,5-dichlorophenyl)-1,4-dihydro-2,6-dimethylpyridine-3,5-dicarboxylate

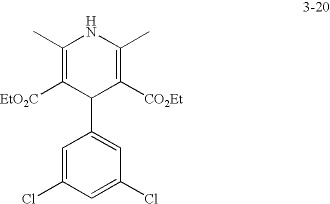

Ethyl acetoacetate (640 μL, 99%, 5.00 mmol) and 3,5-dichlorobenzaldehyde (451 mg, 997%, 2.50 mmol) were taken up in EtOH (500 μL) at rt. NH₄OH (250 μL), the mixture was stirred at ambient temperature for 1 h, then heated to 95° C. After 3 h, the reaction mixture was cooled to ambient temperature, diluted with CH₂Cl₂ (5 mL) and dried over Na₂SO₄. Crystallization from CH₂Cl₂/hexane (1:9) afforded 275 mg (28%) of diethyl 4-(3,5-dichlorophenyl)-1,4-dihydro-2,6-dimethylpyridine-3,5-dicarboxylate as a white solid: MP 102-104° C.; ¹H NMR (500 MHz, CDCl₃) δ 1.25 (t, J=7.1 Hz, 6H), 2.36 (s, 6H), 4.05-4.20 (m, 4H), 4.96 (s, 1H), 5.66 (brs, 1H), 7.13-7.16 (m, 3H); ¹³C NMR (125 MHz, CDCl₃) δ 14.5, 19.9, 40.1, 60.2, 103.5, 126.5, 127.0, 134.4, 144.7, 151.2, 167.3.

### EXAMPLE 38

### Diethyl 4-(2,3-difluorophenyl)-1,4-dihydro-2,6-dimethylpyridine-3,5-dicarboxylate

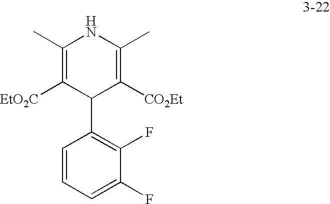

Ethyl acetoacetate (766 μL, 99%, 6.00 mmol) and 2,3-difluorobenzaldehyde (335 μL, 98%, 3.00 mmol) were taken up in EtOH (600 μL) at rt. NH₄OH (300 μL), the mixture was stirred at ambient temperature for 1 h, then heated to 95° C. After 3 h, the reaction mixture was cooled to ambient temperature, diluted with CH₂Cl₂ (10 mL) and dried over Na₂SO₄. Crystallization from CH₂Cl₂/hexane (1:9) afforded 758 mg (69%) of diethyl 4-(2,3-difluorophenyl)-1,4-dihydro-2,6-dimethylpyridine-3,5-dicarboxylate as a white solid: MP 161-162° C.; ¹H NMR (500 MHz, CDCl₃) δ 1.21 (t, J=7.1 Hz, 6H), 2.34 (s, 6H), 4.02-4.13 (m, 4H), 5.28 (s, 1H), 5.72 (brs, 1H), 6.90-6.96 (m, 2H), 7.05-7.10 (m, 1H); ¹³C NMR (125 MHz, CDCl₃) δ 14.0, 19.4, 34.3, 59.8, 102.7, 114.5, 114.6, 123.1, 123.2, 125.6, 125.7, 137.6, 144.5, 167.3.

### EXAMPLE 39

### Di-tert-butyl 4-(2,3-difluorophenyl)-1,4-dihydro-2,6-dimethylpyridine-3,5-dicarboxylate

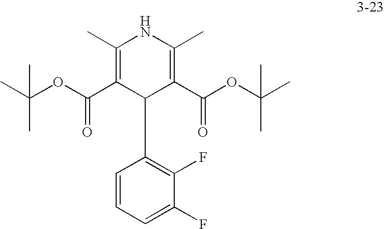

tert-Butyl acetoacetate (988 μL, 99%, 6.00 mmol) and 2,3-difluorobenzaldehyde (335 μL, 98%, 3.00 mmol) were taken up in EtOH (600 μL) at rt. NH₄OH (300 μL) was added, the mixture was stirred at rt 1 h, then the mixture was heated to 95° C. After 3 h, the reaction mixture was cooled to ambient temperature, diluted with CH₂Cl₂ (5 mL) and dried over Na₂SO₄. Crystallization from CH₂Cl₂/hexane (1:9) afforded 644 mg (51%) of di-tert-butyl 4-(2,3-difluorophenyl)-1,4-dihydro-2,6-dimethylpyridine-3,5-dicarboxylate as a white solid: MP 192-194° C.; ¹H NMR (500 MHz, CDCl₃) δ 1.40 (s, 18H), 2.28 (s, 6H), 5.21 (s, 1H), 5.58 (brs, 1H), 6.90-6.96 (m, 2H), 7.05-7.09 (m, 1H); ¹³C NMR (125 MHz, CDCl₃) δ 19.4, 28.2, 35.1, 79.9, 103.8, 114.4, 114.5, 123.0, 123.1, 125.9, 137.2, 137.3, 143.4, 166.8.

### EXAMPLE 40

### Diallyl 4-(4-bromophenyl)-1,4-dihydro-2,6-dimethylpyridine-3,5-dicarboxylate

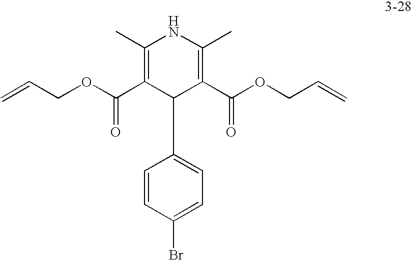

Allyl acetoacetate (1.40 mL, 98%, 10.0 mmol) and 4-bromobenzaldehyde (934 mg, 99%, 5.00 mmol) were taken up in EtOH (1 mL) at rt. NH₄OH (500 μL), the mixture was stirred at ambient temperature for 1 h, then heated to 95° C. After 2 h, the reaction mixture was cooled to ambient temperature, diluted with CH₂Cl₂ (10 mL) and dried over Na₂SO₄. Purification on a column of silica gel (0-10% MeOH/CH₂Cl₂ as eluent) and crystallization from CH₂Cl₂/hexane (1:9) afforded 188 mg (9%) of diallyl 4-(4-bromophenyl)-1,4-dihydro-2,6-dimethylpyridine-3,5-dicarboxylate as a white solid: MP 130-131° C.; ¹H NMR (500 MHz, CDCl₃) δ 2.35 (s, 6H), 4.53-4.61 (m, 4H), 5.04 (s, 1H), 5.17-5.25 (m, 4H), 5.78 (brs, 1H), 5.85-5.93 (m, 2H), 7.16-7.19 (m, 2H), 7.32-7.35 (m, 2H); ¹³C NMR (125 MHz, CDCl₃) δ 19.7, 39.1, 64.6, 103.6, 117.6, 120.0, 129.7, 131.0, 132.6, 144.4, 146.5, 166.9.

### EXAMPLE 41

### Diethyl 4-(3-bromo-4-fluorophenyl)-1,4-dihydro-2,6-dimethylpyridine-3,5-dicarboxylate

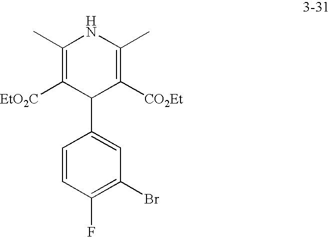

Ethyl acetoacetate (1.28 mL, 99%, 10.0 mmol) and 3-bromo-4-fluorobenzaldehyde (1.03 g, 99%, 5.00 mmol) were taken up in EtOH (1 mL) at rt. NH₄OH (500 μL) was added, the mixture was stirred at rt 1 h, then the mixture was heated to 95° C. After 3 h, the reaction mixture was cooled to ambient temperature, diluted with CH₂Cl₂ (10 mL) and dried over Na₂SO₄. Purification on a column of silica gel (0-10% MeOH/CH₂Cl₂ as eluent) and crystallization from CH₂Cl₂/hexane (1:9) afforded 440 mg (21%) of diethyl 4-(3-bromo-4-fluorophenyl)-1,4-dihydro-2,6-dimethylpyridine-3,5-dicarboxylate as a white solid: MP 118-119° C.; ¹H NMR (500 MHz, CDCl₃) δ 1.25 (t, J=7.1 Hz, 6H), 2.36 (s, 6H), 4.05-4.18 (m, 4H), 4.96 (s, 1H), 5.61 (brs, 1H), 6.97 (t, J=8.5 Hz, 1H), 7.18-7.23 (m, 1H), 7.43-7.46 (m, 1H); ¹³C NMR (125 MHz, CDCl₃) δ 14.2, 19.7, 39.1, 59.9, 103.7, 115.5, 115.7, 128.5, 128.6, 133.0, 144.0, 167.2.

### EXAMPLE 42

### Di-tert-butyl 4-(3-bromo-4-fluorophenyl)-1,4-dihydro-2,6-dimethylpyridine-3,5-dicarboxylate

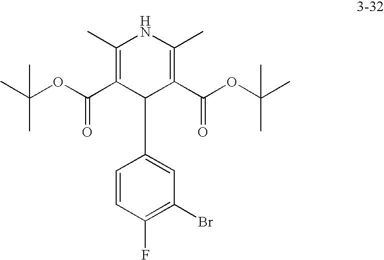

tert-Butyl acetoacetate (1.65 mL, 99%, 10.0 mmol) and 3-bromo-4-fluorobenzaldehyde (1.03 g, 99%, 5.00 mmol) were taken up in EtOH (1 mL) at rt. NH₄OH (500 μL) was added, the mixture was stirred at rt 1 h, then the mixture was heated to 95° C. After 2 h, the reaction mixture was cooled to ambient temperature, diluted with CH₂Cl₂ (10 mL) and dried over Na₂SO₄. Crystallization from CH₂Cl₂/hexane (1:9) afforded 830 mg (34%) of di-tert-butyl 4-(3-bromo-4-fluorophenyl)-1,4-dihydro-2,6-dimethylpyridine-3,5-dicarboxylate as a white solid: MP 171-172° C.; ¹H NMR (500 MHz, CDCl₃) δ 1.43 (s, 18H), 2.32 (s, 6H), 4.88 (s, 1H), 5.44 (brs, 1H), 6.95-7.00 (m, 1H), 7.17-7.21 (m, 1H), 7.44-7.48 (m, 1H); ¹³C NMR (125 MHz, CDCl₃) δ 19.6, 28.3, 39.7, 80.0, 104.9, 115.5, 115.6, 128.4, 128.5, 133.0, 143.0, 166.7.

### EXAMPLE 43

### Diethyl 4-(4-bromo-2-fluorophenyl)-1,4-dihydro-2,6-dimethylpyridine-3,5-dicarboxylate

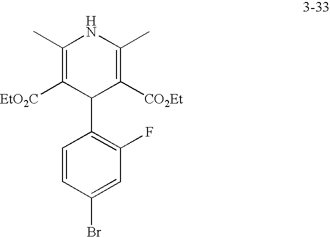

Ethyl acetoacetate (1.28 mL, 99%, 10.0 mmol) and 2-fluoro-4-bromobenzaldehyde (1.06 g, 96%, 5.00 mmol) were taken up in EtOH (1 mL) at rt. NH₄OH (500 μL) was added, the mixture was stirred at rt 1 h, then the mixture was heated to 95° C. After 2 h, the reaction mixture was cooled to ambient temperature, diluted with CH₂Cl₂ (10 mL) and dried over Na₂SO₄. Crystallization from CH₂Cl₂/hexane (1:9) afforded 1.24 g (58%) of diethyl 4-(4-bromo-2-fluorophenyl)-1,4-dihydro-2,6-dimethylpyridine-3,5-dicarboxylate as a white solid: MP 154-155° C.; ¹H NMR (500 MHz, CDCl₃) δ 1.22 (t, J=7.1 Hz, 6H), 2.34 (s, 6H), 4.04-4.11 (m, 4H), 5.21 (s, 1H), 5.61 (brs, 1H), 7.09-7.22 (m, 3H); ¹³C NMR (125 MHz, CDCl₃) δ 14.0, 19.5, 34.2, 59.8, 102.7, 118.4, 118.6, 119.7, 119.8, 126.9, 132.3, 132.4, 134.2, 134.3, 144.3, 167.2.

### EXAMPLE 44

### Di-tert-butyl 4-(4-bromo-2-fluorophenyl)-1,4-dihydro-2,6-dimethylpyridine-3,5-dicarboxylate

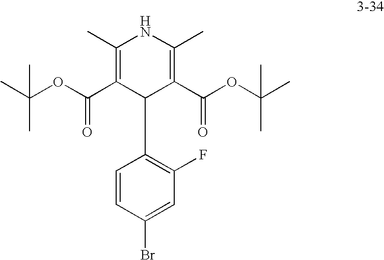

tert-Butyl acetoacetate (1.65 mL, 99%, 10.0 mmol) and 2-fluoro-4-bromobenzaldehyde (1.06 g, 96%, 5.00 mmol) were taken up in EtOH (1 mL) at rt. NH₄OH (500 μL) was added, the mixture was stirred at rt 1 h, then the mixture was heated to 95° C. After 2 h, the reaction mixture was cooled to ambient temperature, diluted with CH₂Cl₂ (10 mL) and dried over Na₂SO₄. Crystallization from CH₂Cl₂/hexane (1:9) afforded 672 mg (28%) of di-tert-butyl 4-(4-bromo-2-fluorophenyl)-1,4-dihydro-2,6-dimethylpyridine-3,5-dicarboxylate as a white solid: MP 187-188° C.; ¹H NMR (500 MHz, CDCl₃) δ 1.41 (s, 18H), 2.29 (s, 6H), 5.15 (s, 1H), 5.47 (brs, 1H), 7.10-7.22 (m, 3H); ¹³C NMR (125 MHz, CDCl₃) δ 19.5, 28.2, 34.8, 79.9, 103.8, 118.4, 118.6, 126.8, 132.4, 132.5, 143.3, 166.8.

### EXAMPLE 45

### Bis(2-methoxyethyl) 4-(3-bromophenyl)-1,4-dihydro-2,6-dimethylpyridine-3,5-dicarboxylate

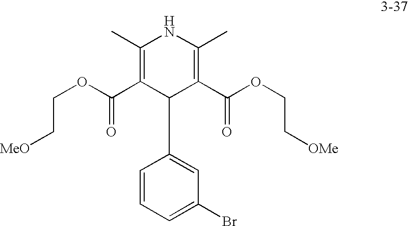

2-Methoxyethyl acetoacetate (1.51 mL, 97%, 10.0 mmol) and 3-bromobenzaldehyde (610 μL, 96%, 5.00 mmol) were taken up in EtOH (1 mL) at rt. NH₄OH (500 μL) was added, the mixture was stirred at rt 1 h, then the mixture was heated to 95° C. After 3 h, the reaction mixture was cooled to ambient temperature, diluted with CH₂Cl₂ (10 mL) and dried over Na₂SO₄. Crystallization from CH₂Cl/hexane (1:9) to afford 1.79 g (76%) of bis(2-methoxyethyl) 4-(3-bromophenyl)-1,4-dihydro-2,6-dimethylpyridine-3,5-dicarboxylate as a white solid: MP 124.5-125.5° C.; ¹H NMR (500 MHz, CDCl₃) δ 2.36 (s, 6H), 3.39 (s, 6H), 3.55-3.59 (m, 4H), 4.13-4.19 (m, 2H), 4.21-4.26 (m, 2H), 5.01 (s, 1H), 5.67 (brs, 1H), 7.07-7.12 (m, 1H), 7.25-7.29 (m, 2H), 7.43-7.46 (m, 1H); ¹³C NMR (125 MHz, CDCl₃) δ 19.7, 39.6, 58.9, 62.9, 70.5, 103.6, 121.9, 126.9, 129.2, 129.5, 131.2, 144.5, 149.9, 167.1.

### EXAMPLE 46

### Bis(2-methoxyethyl) 4-(4-bromophenyl)-1,4-dihydro-2,6-dimethylpyridine-3,5-dicarboxylate

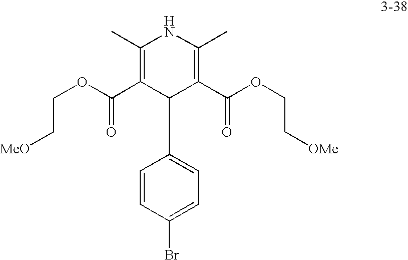

2-Methoxyethyl acetoacetate (1.51 mL, 97%, 10.0 mmol) and 4-bromobenzaldehyde (934 mg, 99%, 5.00 mmol) were taken up in EtOH (1 mL) at rt. NH₄OH (500 μL) was added, the mixture was stirred at rt 1 h, then the mixture was heated to 95° C. After 3 h, the reaction mixture was cooled to ambient temperature, diluted with CH₂Cl₂ (10 mL) and dried over Na₂SO₄. Crystallization from CH₂Cl/hexane (1:9) to afford 1.50 g (64%) of bis(2-methoxyethyl) 4-(4-bromophenyl)-1,4-dihydro-2,6-dimethylpyridine-3,5-dicarboxylate as a white solid: MP 116-117° C.; ¹H NMR (500 MHz, CDCl₃) δ 2.35 (s, 6H), 3.37 (s, 6H), 3.51-3.60 (m, 4H), 4.14-4.19 (m, 2H), 4.20-4.26 (m, 2H), 5.01 (s, 1H), 5.62 (brs, 1H), 7.19-7.23 (m, 2H), 7.32-7.36 (m, 1H); ¹³C NMR (125 MHz, CDCl₃) δ 19.7, 39.3, 58.8, 62.8, 70.6, 103.7, 119.9, 129.9, 130.9, 144.2, 146.6, 167.2.

### EXAMPLE 47

### Diethyl 4-(5-bromo-2-fluorophenyl)-1,4-dihydro-2,6-dimethylpyridine-3,5-dicarboxylate

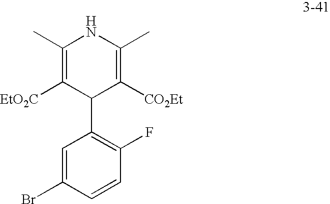

Ethyl acetoacetate (1.28 mL, 99%, 10.0 mmol) and 2-fluoro-5-bromobenzaldehyde (615 μL, 97%, 5.00 mmol) were taken up in EtOH (1 mL) at rt. NH₄OH (500 μL) was added, the mixture was stirred at rt 1 h, 80° C. 1 h, then the mixture was heated to 95° C. After 1.5 h, the reaction mixture was cooled to ambient temperature, diluted with CH₂Cl₂ (10 mL) and dried over Na₂SO₄. Crystallization from CH₂Cl₂/hexane (1:9) afforded 1.01 g (47%) of diethyl 4-(5-bromo-2-fluorophenyl)-1,4-dihydro-2,6-dimethylpyridine-3,5-dicarboxylate as a white solid: MP 118-119° C.; ¹H NMR (500 MHz, CDCl₃) δ 1.20-1.25 (m, 6H), 2.34-2.36 (m, 6H), 4.02-4.14 (m, 4H), 5.21 (s, 1H), 5.69 (brs, 1H), 6.81-6.84 (m, 1H), 7.20-7.24 (m, 1H), 7.37-7.41 (m, 1H); ¹³C NMR (125 MHz, CDCl₃) δ 14.0, 14.1, 19.5, 22.6, 31.6, 34.5, 59.8, 102.5, 116.0, 116.7, 116.9, 130.5, 130.6, 134.0, 137.1, 137.2, 144.6, 158.0, 160.0, 167.2.

### EXAMPLE 48

### Di-tert-butyl 4-(5-bromo-2-fluorophenyl)-1,4-dihydro-2,6-dimethylpyridine-3,5-dicarboxylate

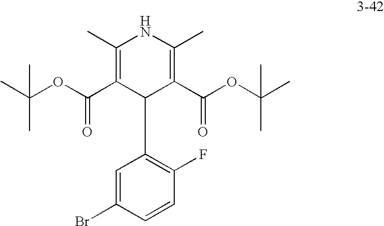

tert-Butyl acetoacetate (1.65 mL, 99%, 10.0 mmol) and 2-fluoro-5-bromobenzaldehyde (615 μL, 97%, 5.00 mmol) were taken up in EtOH (1 mL) at rt. NH₄OH (500 μL) was added, the mixture was stirred at rt 1 h, 80° C. 1 h, then the mixture was heated to 95° C. After 2 h, the reaction mixture was cooled to ambient temperature, diluted with CH₂Cl₂ (10 mL) and dried over Na₂SO₄. Crystallization from CH₂Cl₂/hexane (1:9) afforded 51 mg (2%) of di-tert-butyl 4-(5-bromo-2-fluorophenyl)-1,4-dihydro-2,6-dimethylpyridine-3,5-dicarboxylate as a white solid: MP 173-174° C.; ¹H NMR (500 MHz, CDCl₃) δ 1.40-1.43 (m, 18H), 2.29-2.31 (m, 6H), 5.11-5.13 (m, 1H), 5.54 (brs, 1H), 6.80-6.86 (m, 1H), 7.20-7.25 (m, 1H), 7.38-7.42 (m, 1H); ¹³C NMR (125 MHz, CDCl₃) δ 19.5, 28.2, 35.6, 79.9, 103.5, 115.8, 116.8, 117.0, 130.4, 134.2, 134.3, 136.6, 136.8, 143.6, 158.2, 160.2, 166.7.

### EXAMPLE 49

### Diethyl 4-(3-fluorophenyl)-1,4-dihydro-2,6-dimethylpyridine-3,5-dicarboxylate

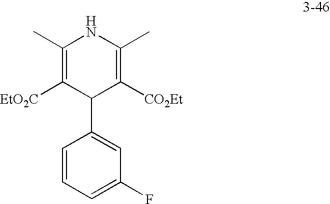

Ethyl acetoacetate (1.28 mL, 99%, 10.0 mmol) and 3-fluorobenzaldehyde (542 μL, 97%, 5.00 mmol) were taken up in EtOH (1 mL) at rt. NH₄OH (500 μL) was added, the mixture was stirred at rt 1 h, then the mixture was heated to 95° C. After 3 h, the reaction mixture was cooled to ambient temperature, diluted with CH₂Cl₂ (10 mL) and dried over Na₂SO₄. Crystallization from CH₂Cl₂/hexane (1:9) afforded 1.22 g (70%) of diethyl 4-(3-fluorophenyl)-1,4-dihydro-2,6-dimethylpyridine-3,5-dicarboxylate as a white solid: MP 149-150° C.; ¹H NMR (500 MHz, CDCl₃) δ 1.23 (t, J=7.1 Hz, 6H), 2.35 (s, 6H), 4.05-4.17 (m, 4H), 4.98 (s, 1H), 5.66 (brs, 1H), 6.87-6.92 (m, 2H), 7.23-7.27 (m, 2H); ¹³C NMR (125 MHz, CDCl₃) δ 14.2, 19.6,39.0,59.8, 104.2, 114.4, 114.6, 129.4, 129.5, 143.6, 143.7, 160.4, 162.3, 167.5.

### EXAMPLE 50

### Di-tert-butyl 4-(3-fluorophenyl)-1,4-dihydro-2,6-dimethylpyridine-3,5-dicarboxylate

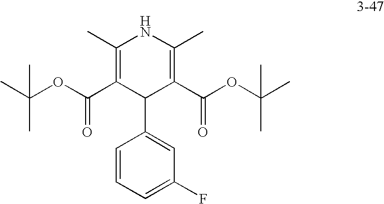

tert-Butyl acetoacetate (1.65 mL, 99%, 10.0 mmol) and 3-fluorobenzaldehyde (542 μL, 97%, 5.00 mmol) were taken up in EtOH (1 mL) at rt. NH₄OH (500 μL) was added, the mixture was stirred at rt 1 h, then the mixture was heated to 95° C. After 3 h, the reaction mixture was cooled to ambient temperature, diluted with CH₂Cl₂ (10 mL) and dried over Na₂SO₄. Crystallization from CH₂Cl₂/hexane (1:9) afforded 368 mg (21%) of di-tert-butyl 4-(3-fluorophenyl)-1,4-dihydro-2,6-dimethylpyridine-3,5-dicarboxylate as a white solid: MP 178-179° C.; ¹H NMR (500 MHz, CDCl₃) δ 1.42 (s, 18H), 2.31 (s, 6H), 4.94 (s, 1H), 5.52 (brs, 1H), 6.80-6.86 (m, 1H), 6.95-7.01 (m, 1H), 7.06-7.10 (m, 1H), 7.14-7.20 (m, 1H); ¹³C NMR (125 MHz, CDCl₃) δ 19.5, 28.3, 40.1, 79.8, 104.9, 112.6, 112.8, 114.6, 114.8, 123.5, 123.6, 128.9, 143.0, 150.4, 150.5, 161.7, 163.7, 166.8.

### EXAMPLE 51

### Diethyl 4-(4-fluorophenyl)-1,4-dihydro-2,6-dimethylpyridine-3,5-dicarboxylate

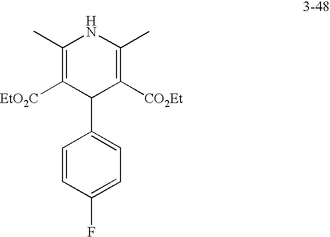

Ethyl acetoacetate (1.28 mL, 99%, 10.0 mmol) and 4-fluorobenzaldehyde (551 μL, 98+%, 5.00 mmol) were taken up in EtOH (1 mL) at rt. NH₄OH (500 μL) was added, the mixture was stirred at rt 1 h, then the mixture was heated to 95° C. After 3 h, the reaction mixture was cooled to ambient temperature, diluted with CH₂Cl₂ (10 mL) and dried over Na₂SO₄. Crystallization from CH₂Cl₂/hexane (1:9) afforded 1.21 g (58%) of diethyl 4-(4-fluorophenyl)-1,4-dihydro-2,6-dimethylpyridine-3,5-dicarboxylate as a white solid: MP 150-151° C.; ¹H NMR (500 MHz, CDCl₃) δ 1.23 (t, J=7.1 Hz, 6H), 2.34 (s, 6H), 4.05-4.17 (m, 4H), 4.98 (s, 1H), 5.72 (brs, 1H), 6.88-6.92 (m, 2H), 7.22-7.27 (m, 2H); ¹³C NMR (125 MHz, CDCl₃) δ 14.2, 19.6, 39.0, 59.8, 104.1, 114.4, 114.6, 129.4, 129.5, 143.6, 143.7, 143.8, 160.4, 162.3, 167.5.

### EXAMPLE 52

### Di-tert-butyl 4-(4-fluorophenyl)-1,4-dihydro-2,6-dimethylpyridine-3,5-dicarboxylate

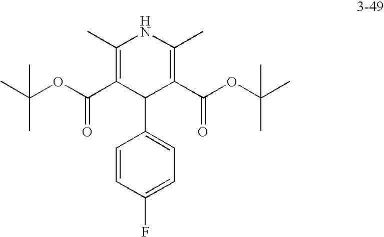

tert-Butyl acetoacetate (1.65 mL, 99%, 10.0 mmol) and 4-fluorobenzaldehyde (551 μL, 98+%, 5.00 mmol) were taken up in EtOH (1 mL) at rt. NH₄OH (500 μL) was added, the mixture was stirred at rt 1 h, then the mixture was heated to 95° C. After 3 h, the reaction mixture was cooled to ambient temperature, diluted with CH₂Cl₂ (10 mL) and dried over Na₂SO₄. Crystallization from CH₂Cl₂/hexane (1:9) afforded 658 mg (38%) of di-tert-butyl 4-(4-fluorophenyl)-1,4-dihydro-2,6-dimethylpyridine-3,5-dicarboxylate as a white solid: MP 149-150° C.; ¹H NMR (500 MHz, CDCl₃) δ 1.41 (s, 18H), 2.30 (s, 6H), 4.91 (s, 1H), 5.48 (brs, 1H), 6.88-6.93 (m, 2H), 7.22-7.27 (m, 2H); ¹³C NMR (125 MHz, CDCl₃) δ 19.5, 28.3,39.6, 79.7, 105.4, 114.2, 114.4, 129.4, 142.7, 143.8, 160.3, 162.2, 166.9.

### EXAMPLE 53

### Dimethyl 4-(4-bromophenyl)-1,4-dihydro-2,6-bis(methoxymethyl)pyridine-3,5-dicarboxylate

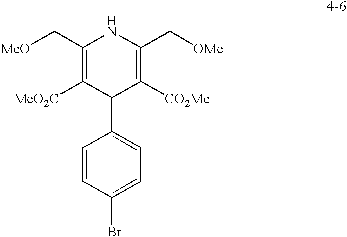

Methyl 4-methoxyacetoacetate (4.14 mL, 97%, 30.0 mmol) and 4-bromobenzaldehyde (1.87 g, 99%, 10.0 mmol) were taken up in EtOH (5 mL) at rt. NH₄OH (1.5 mL) was added, the mixture was stirred at rt 30 min, 50° C. 1.5 h, then the mixture was heated to 95° C. After 24 h, the reaction mixture was cooled to ambient temperature, diluted with CH₂Cl₂ (20 mL) and dried over Na₂SO₄. Crystallization from EtOAc/hexane (1:9) to afford 2.32 g (53%) of dimethyl 4-(4-bromophenyl)-1,4-dihydro-2,6-bis(methoxymethyl)pyridine-3,5-dicarboxylate as a white solid: MP 162-163° C.; ¹H NMR (500 MHz, CDCl₃) δ 3.49 (s, 6H), 3.65 (s, 6H), 4.64 (d, J=16.1 Hz, 2H), 4.73 (d, J=16.1 Hz, 2H), 4.97 (s, 1H), 7.13-7.17 (m, 2H), 7.33-7.37 (m, 2H), 8.40 (brs, 1H); ¹³C NMR (125 MHz, CDCl₃) δ 38.9, 51.0, 59.1, 69.8, 101.1, 120.1, 129.5, 131.1, 145.4, 146.3, 167.3.

### EXAMPLE 54

### Diethyl 1-benzyl-4-(4-bromophenyl)-1,4-dihydro-2,6-dimethylpyridine-3,5-dicarboxylate

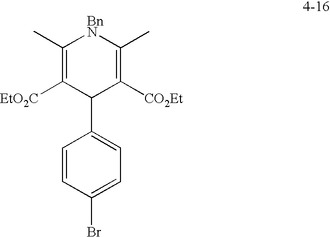

Diethyl 4-(4-bromophenyl)-1,4-dihydro-2,6-dimethylpyridine-3,5-dicarboxylate [CML-3-1] (400 mg, 0.980 mmol) was added to a stirring suspension of NaH (59 mg, 60% dispersion in mineral oil, 1.5 eq.) in DMF (15 mL). After 30 min at rt under N₂, benzyl chloride (567 mL, 5.98 mmol) was added dropwise via syringe and the mixture was stirred at rt under N₂. After 18 h, the entire reaction mixture was added to a separatory funnel along with 50% aqueous NH₄Cl (25 mL). The aqueous suspension was extracted with EtOAc (30 mL) and the organic extract was washed with water (2×20 mL), dried over Na₂SO₄, filtered and concentrated under reduced pressure. Purification on a column of silica gel (0-10% EtOAc/hexane as eluent) and crystallization from EtOAc/hexane (1:9) afforded 17 mg (4%) of diethyl 1-benzyl4-(4-bromophenyl)-1,4-dihydro-2,6-dimethylpyridine-3,5-dicarboxylate as a white solid: MP 168-169° C.; ¹H NMR (500 MHz, CDCl₃) δ 1.28 (t, J=7.1 Hz, 6H), 2.46 (s, 6H), 4.18 (q, J=7.1 Hz, 4H), 4.87 (s, 2H), 5.32 (s, 1H), 6.93-6.97 (m, 2H), 7.05-7.08 (m, 2H), 7.25-7.28 (m, 4H), 7.30-7.33 (m, 2H); ¹³C NMR (125 MHz, CDCl₃) δ 14.3, 16.8, 38.0, 49.4, 60.1, 106.7, 119.8, 126.0, 127.5, 128.8, 129.2, 130.9, 137.6, 145.6, 148.8, 168.0.

### EXAMPLE 55

### Di-tert-butyl 1,4-dihydro-2,6-dimethyl-4-(2,4-dimethylphenyl)pyridine-3,5-dicarboxylate

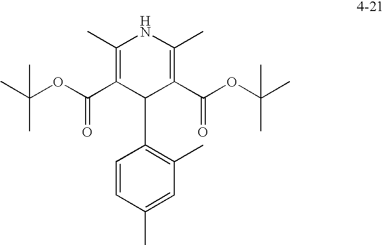

tert-Butyl acetoacetate (988 μL, 99%, 6.00 mmol) and 2,4-dimethylbenzaldehyde (271 mg, 99%, 2.00 mmol) were taken up in EtOH (1 mL) at rt. NH₄OH (300 μL) was added, the mixture was stirred at rt 1 h, 50° C. 1 h, then the mixture was heated to 95° C. After 16 h, the reaction mixture was cooled to ambient temperature, diluted with CH₂Cl₂ (10 mL) and dried over Na₂SO₄. Crystallization from CH₂Cl₂/hexane (1:9) afforded 158 mg (19%) of di-tert-butyl 1,4-dihydro-2,6-dimethyl-4-(2,4-dimethylphenyl)pyridine-3,5-dicarboxylate as a white solid: MP 197-198° C.; ¹H NMR (500 MHz, CDCl₃) δ 1.40 (s, 18H), 2.24 (s, 9H), 2.46 (s, 3H), 5.12 (s, 1H), 5.39 (brs, 1H), 6.83-6.873 (m, 2H), 7.11-7.15 (m, 1H); ¹³C NMR (125 MHz, CDCl₃) δ 19.6, 19.8, 20.9, 28.3, 37.1, 79.7, 105.9, 126.3, 129.8, 130.8, 135.1, 141.2, 143.2, 167.5.

### EXAMPLE 56

### 3-Ethyl 5-methyl 4-(2-chlorophenyl)-1,4-dihydro-2,6-dimethylpyridine-3,5-dicarboxylate

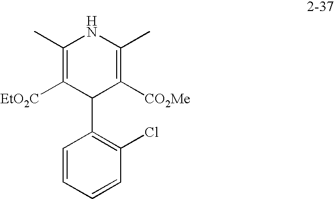

Ethyl acetoacetate (638 μL, 99%, 5.00 mmol), 2-chlorobenzaldehyde (562 μL, 99%, 5.00 mmol) and methyl-3-aminocrotonate (593 mg, 97%, 5.00 mmol) were taken up in EtOH (3.25 mL) at rt. AcOH (217 μL) was added and the mixture was heated to 95° C. After 3 h, the reaction mixture was cooled to ambient temperature, diluted with EtOAc (20 mL), dried over Na₂SO₄ and crystallized from EtOAc/hexane (1:9) to afford 451 mg (26%) of 3-ethyl 5-methyl 4-(2-chlorophenyl)-1,4-dihydro-2,6-dimethylpyridine-3,5-dicarboxylate as a white solid: MP 125-127° C.; ¹H NMR (500 MHz, CDCl₃) δ 1.20 (t, J=7.0 Hz, 6H), 2.30 (s, 3H), 2.31 (s, 3H), 3.61-3.62 (m, 3H), 4.05-4.10 (m, 4H), 5.40 (s, 1H), 5.70-5.74 (m, 1H), 7.02-7.06 (m, 1H), 7.10-7.15 (m, 1H), 7.22-7.25 (m, 1H), 7.35-7.39 (m, 1H); ¹³C NMR (125 MHz, CDCl₃) δ 14.3, 19.4, 19.5, 19.6, 37.2, 37.3, 37.6, 50.8, 50.9, 59.8, 103.8, 103.9, 104.1, 126.7, 126.8, 126.9, 127.3, 129.3, 131.2, 131.4, 131.6, 132.4, 143.9, 144.0, 144.1, 145.6, 145.8, 145.9, 167.6, 167.7, 168.0, 168.1; MS (ES) m/z 372 (M+Na)⁺, 350 (M+H)⁺, 318, 304, 272,238; m/z 350.098 (calcd for C₁₈H₂₁ClNO₄ (M+H)⁺: 350.115).

### EXAMPLE 57

### Methyl 5-acetyl-4-(2-chlorophenyl)-1,4-dihydro-2,6-dimethylpyridine-3-carboxylate

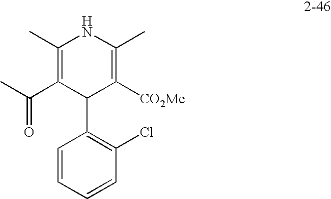

2,4-Pentanedione (519 μL, 99+%, 5.00 mmol), 2-chlorobenzaldehyde (562 μL, 99%, 5.00 mmol) and methyl-3-aminocrotonate (593 mg, 97%, 5.00 mmol) were taken up in EtOH (3.25 mL) at rt. AcOH (217 μL) was added and the mixture was heated to 95° C. After 3 h, the reaction mixture was cooled to ambient temperature, taken up in EtOAc (20 mL), dried over Na₂SO₄ and crystallized from EtOAc/hexane (1:9) to afford 176 mg (11%) of methyl 5-acetyl-4-(2-chlorophenyl)-1,4-dihydro-2,6-dimethylpyridine-3-carboxylate as a white solid: MP 183-184° C.; ¹H NMR (500 MHz, CDCl₃) δ 2.25-2.27 (m, 3H), 2.29-2.32 (m, 6H), 3.60-3.67 (m, 3H), 5.39-5.44 (m, 1H), 5.77-5.92 (m, 1H), 7.01-7.09 (m, 1H), 7.10-7.16 (m, 1H), 7.21-7.27 (m, 1H), 7.32-7.38 (m, 1H); ¹³C NMR (125 MHz, CDCl₃) δ 19.4, 20.1, 29.9, 37.2, 37.8, 50.8, 50.9, 103.9, 104.1, 112.9, 126.9, 127.3, 127.4, 127.8, 129.2, 129.6, 131.2, 131.3, 132.4, 142.3, 143.7, 144.1, 144.9, 145.9, 168.0, 199.7; MS (ES) m/z 358 (M+K)⁺, 318 (M−H)⁺, 304 (M−CH₃), 290, 272, 224; m/z 358.063 (calcd for C₁₇H₁₈ClKNO₃ (M+K)⁺: 358.061).

### EXAMPLE 58

### 3-Ethyl 5-methyl 4-(2-bromophenyl)-1,4-dihydro-2,6-dimethylpyridine-3,5-dicarboxylate

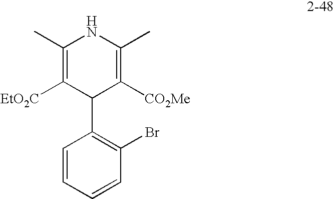

Ethyl acetoacetate (638 μL, 99%, 5.00 mmol), 2-bromobenzaldehyde (604 μL, 97%, 5.00 mmol) and methyl-3-aminocrotonate (593 mg, 97%, 5.00 mmol) were taken up in EtOH (3.25 mL) at rt. AcOH (217 μL) was added and the mixture was heated to 95° C. After 3 h, the reaction mixture was cooled to ambient temperature, taken up in EtOAc (20 mL), dried over Na₂SO₄ and crystallized from EtOAc/hexane (1:9) to afford 584 mg (30%) of 3-ethyl 5-methyl 4-(2-bromophenyl)-1,4-dihydro-2,6-dimethylpyridine-3,5-dicarboxylate as a white solid: MP 134.5-135.5° C.; ¹H NMR (500 MHz, CDCl₃) δ 1.20 (t, J=7.1 Hz, 3H), 2.28-2.32 (m, 6H), 3.62-3.64 (m, 3H), 4.05-4.16 (m, 2H), 5.36 (s, 1H), 5.71 (brs, 1H), 6.93-6.97 (m, 1H), 7.14-7.19 (m, 1H), 7.36-7.40 (m, 1H), 7.41-7.44 (m, 1H); ¹³C NMR (125 MHz, CDCl₃) δ 14.4, 19.4, 19.5, 39.4, 39.5, 39.8, 50.8, 59.7, 59.8, 104.1, 104.2, 104.3, 104.5, 122.6, 127.4, 127.6, 127.7, 131.2, 131.4, 131.6, 132.6, 132.7, 143.6, 143.7, 143.8, 143.9, 147.4, 147.7, 147.9, 167.6, 167.7, 168.0, 168.1; MS (ES) m/z 416 (M+Na)⁺, 394 (M−H)⁺, 380, 364, 347, 317, 282, 268; m/z 394.052 (calcd for C₁₈H₂₁BrNO₄ (M+H)⁺: 394.065).

### EXAMPLE 59

### Methyl 5-acetyl-4-(2-bromophenyl)-1,4-dihydro-2,6-dimethylpyridine-3-carboxylate

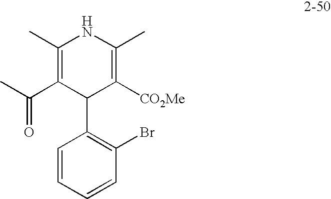

2,4-Pentanedione (519 μL, 99+%, 5.00 mmol), 2-bromobenzaldehyde (604 μL, 97%, 5.00 mmol) and methyl-3-aminocrotonate (593 mg, 97%, 5.00 mmol) were taken up in EtOH (3.25 mL) at rt. AcOH (217 μL) was added and the mixture was heated to 95° C. After 3 h, the reaction mixture was cooled to ambient temperature, taken up in EtoAc (20 mL) and dried over Na₂SO₄. The residue was purified on a column of silica gel (0-10% MeOH/CH₂Cl₂) and crystallized from CH₂Cl₂/hexane (1:20) to afford 121 mg (7%) of methyl 5-acetyl4-(2-bromophenyl)-1,4-dihydro-2,6-dimethylpyridine-3-carboxylate as a pale yellow solid: MP 146-148° C.; ¹H NMR (500 MHz, CDCl₃) δ 2.24-2.32 (m, 6H), 3.63 (s, 3H), 3.68 (s, 3H), 5.35-5.38 (m, 1H), 5.73-5.83 (m, 1H), 6.93-7.00 (m, 1H), 7.15-7.20 (m, 1H), 7.33-7.39 (m, 1H), 7.41-7.45 (m, 1H); ¹³C NMR (125 MHz, CDCl₃) δ 19.3, 19.4, 20.0, 30.4, 39.3, 40.0, 50.8, 50.9, 104.2, 104.3, 113.4, 121.5, 122.6, 127.5, 127.7, 128.1, 131.2, 131.2, 132.6, 133.0, 141.9, 143.5, 144.0, 146.8, 147.9, 168.0, 199.9; MS (ES) m/z 386 (M+Na)⁺, 364 (M−H)⁺, 348, 332, 252, 224, 208; m/z 364.034 (calcd for C₁₇H₁₉BrNO₃ (M+H)⁺: 364.054).

It should be understood that the embodiments described herein are for illustrative purposes only and that various modifications or changes in light thereof will be suggested to persons skilled in the art and are to be included within the spirit and purview of this application.

1-26. (canceled)

27. A method for diagnosing a disease associated with cerebral accumulation of Alzheimer's amyloid in an animal or human, comprising:
taking a first measurement of plasma, urine, serum, whole blood, or cerebral spinal fluid (CSF) concentration of β-amyloid or fragment thereof in the peripheral circulation of the animal or human;
administering to the animal or human a diagnostically effective amount of a compound that decreases capacitative calcium entry by at least about 10% or more in cells;
taking a second measurement of plasma, serum, whole blood, urine or CSF concentration of β-amyloid in the peripheral circulation of the animal or human; and
calculating the difference between the first measurement and the second measurement, wherein a change in the plasma, serum, whole blood, urine or CSF concentration of β-amyloid in the second measurement compared to the first measurement indicates a possible diagnosis of a disease associated with cerebral accumulation of Alzheimer's amyloid in the animal or human;
wherein the compound is selected from the group consisting of SKF96365, econazole, clotrimazole, SR 33805, loperamide, tetrandrine, R24571, amlodipine, nitrendipine, MRS 1845, tyrphostin A9, BTB 14328, CD 04170, HTS 01512, HTS 07578, HTS 10306, JFD 01209, JFD 03266, JFD 03274, JFD 03282, JFD 03292, JFD 03293, JFD 03294, JFD 03305, JFD 03311, JFD 03318, PD 00463, RJC 03403, RJC 03405, RJC 03413, RJC 03423, SEW 02070, XBX 00343, R-niguldipine, (S)-(+)-niguldipine, artemisinin, celastrol, quinazoline, isohelenin, kamebakaurin, parthenolide, IKK-2 Inhibitor IV and derivatives thereof.

28. The method of claim 27, wherein the compound decreases capacitative calcium entry by at least about 10% or more in cells selected from Chinese hamster ovary cells that overexpress APP751, or selected from human neuronal precursor cells (HNPC); primary culture of human astrocytes; neuroblastoma cells; human brain microvascular endothelial primary culture; or human umbilical cord endothelial cells (HUVEC).

29. A method for diagnosing a disease associated with cerebral accumulation of Alzheimer's amyloid in an animal or human, comprising:
taking a first measurement of plasma, urine, serum, whole blood, or cerebral spinal fluid (CSF) concentration of β-amyloid in the peripheral circulation of the animal or human;
administering to the animal or human a diagnostically effective amount of a compound that decreases capacitative calcium entry by at least about 10% or more in cells;
taking a second measurement of plasma, serum, whole blood, urine or CSF concentration of β-amyloid in the peripheral circulation of the animal or human; and
calculating the difference between the first measurement and the second measurement, wherein a change in the plasma, serum, whole blood, urine or CSF concentration of β-amyloid in the second measurement compared to the first measurement indicates a possible diagnosis of a disease associated with cerebral accumulation of Alzheimer's amyloid in the animal or human;
wherein the compound is a dihydropyridine which is optionally other than nilvadipine, nimodipine or nitrendipine or salt or free base thereof; an imidazole compound; an isoquinoline alkaloid compound; a calmodulin-mediated enzyme activation inhibitor; an inhibitor of kinase activity of the platelet-derived growth factor (PDGF) receptor; an NF-kB activation inhibitor; diterpene or triterpene compound; a quinazoline compound; a sesquiterpene lactone; or an inhibitor of IKK-2; and
wherein the compound decreases capacitative calcium entry by at least about 10% or more in cells.

30. A method for diagnosing a disease associated with cerebral accumulation of Alzheimer's amyloid in an animal or human, comprising:
taking a first measurement of plasma, urine, serum, whole blood, or cerebral spinal fluid (CSF) concentration of β-amyloid in the peripheral circulation of the animal or human;
administering to the animal or human a diagnostically effective amount of a compound that decreases capacitative calcium entry by at least about 10% or more in cells;
taking a second measurement of plasma, serum, whole blood, urine or CSF concentration of β-amyloid in the peripheral circulation of the animal or human; and
calculating the difference between the first measurement and the second measurement, wherein a change in the plasma, serum, whole blood, urine or CSF concentration of β-amyloid in the second measurement compared to the first measurement indicates a possible diagnosis of a disease associated with cerebral accumulation of Alzheimer's amyloid in the animal or human;
wherein the compound is a compound of Formula I, or a salt, ester or prodrug thereof, or an R or S isomers thereof: 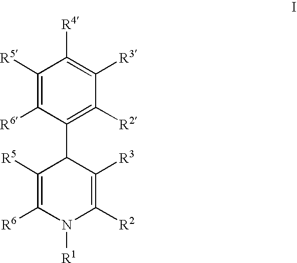
wherein:
R¹ is H, alkyl, optionally substituted aryl, optionally substituted heterocycle, alkyl or aryl ether;
R² and R⁶ are independently alkyl, alkyl ether, aryl ether, halogen, or hydroxy;
R³ and R⁵ are independently optionally substituted alkyl ester, aryl ester, silyl ester, alkyl amide, aryl amide, cyano, or nitro;
R^{2′} and R^{6′} are independently H, alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle;
R^{3′} and R^{5′} are independently H, alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle;
R^{4′} is independently H, alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle;
or R^{2′} and R^{3′} together can optionally form a 4, 5, 6 or 7 membered heterocycle containing 1, 2, or 3 heteratoms and can be optionally substituted with alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle;
or R^{3′} and R^{4′} together can optionally form a 4, 5, 6 or 7 membered heterocycle containing 1, 2, or 3 heteratoms and can be optionally substituted with alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle;
or R^{4′} and R^{5′} together can optionally form a 4, 5, 6 or 7 membered heterocycle containing 1, 2, or 3 heteratoms and can be optionally substituted with alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle;
or R^{5′} and R^{6′} together can optionally form a 4, 5, 6 or 7 membered heterocycle containing 1, 2, or 3 heteratoms and can be optionally substituted with alkyl, optionally substituted alkyl ether, optionally substituted aryl ether, halogen, hydroxy, nitro, carboxylic acid, boronic acid, haloalkyl, amine, optionally substituted alkyl amine, nitrile, optionally substituted alkyl thioether, optionally substituted aryl thioether, or optionally substituted heterocycle.

31. The method of claim 30, wherein, in the compound of Formula I:
R¹ is H, alkyl, optionally substituted aryl, optionally substituted heterocycle, alkyl or aryl ether;
R² and R⁶ are independently alkyl, alkyl ether, aryl ether, halogen, or hydroxy;
R³ and R⁵ are independently alkyl ester, aryl ester, silyl ester, alkyl amide, aryl amide, cyano, or nitro;
R^{2′} and R^{6′} are independently H, optionally substituted alkyl, alkyl ether, aryl ether, halogen, hydroxy, nitro, or optionally substituted heterocycle;
R^{3′} and R^{5′} are independently H, optionally substituted alkyl, alkyl ether, aryl ether, halogen, hydroxy, nitro, or optionally substituted heterocycle;
R^{4′} is independently H, alkyl, alkyl ether, aryl ether, halogen, hydroxy, nitro, or optionally substituted heterocycle.

32. The method of claim 30, wherein, in the compound of Formula I:
R¹ is H;
R² and R⁶ are independently alkyl;
R³ and R⁵ are independently cyano or alkyl ester;
R^{2′} and R^{6′} are independently H, halo, or nitro;
R^{3′} and R^{5′} are independently H or halo; and
R^{4′} is independently H, alkyl, alkyl ether, halo, or nitro.

33. The method of claim 30, wherein, in the compound of Formula I:
R¹ is H;
R² and R⁶ are independently alkyl;
R³ and R⁵ are independently alkyl ester, wherein, in at least one of R₂ and R₃ the alkyl of the alkyl ester comprises 10-30 carbon atoms;
R^{2′}, R^{3′}, R^{4′}, R⁶′ are independently H, halo, or nitro.

34. The method of claim 30, wherein, in the compound of Formula I:
R¹ is H;
R² and R⁶each are alkyl;
R³ and R⁵ each are C(O)Oalkyl;
R^{2′} and R^{6′} are independently H, F, Br, or nitro;
R^{3′} and R^{5′} each are H;
R^{4′} is H or halo.

35. A method for diagnosing a disease associated with cerebral accumulation of Alzheimer's amyloid in an animal or human, comprising:
taking a first measurement of plasma, urine, serum, whole blood, or cerebral spinal fluid (CSF) concentration of β-amyloid in the peripheral circulation of the animal or human;
administering to the animal or human a diagnostically effective amount of a compound that decreases capacitative calcium entry by at least about 10% or more in cells;
taking a second measurement of plasma, serum, whole blood, urine or CSF concentration of β-amyloid in the peripheral circulation of the animal or human; and
calculating the difference between the first measurement and the second measurement, wherein a change in the plasma, serum, whole blood, urine or CSF concentration of β-amyloid in the second measurement compared to the first measurement indicates a possible diagnosis of a disease associated with cerebral accumulation of Alzheimer's amyloid in the animal or human;
wherein the compound is a compound of formula VI, or a salt, ester or prodrug there of, or an R or S isomer thereof: 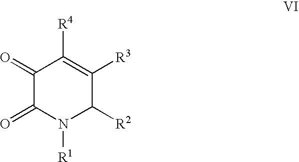
wherein:
R¹ is C3-12 alkyl, and is optionally cycloalkyl, cyclohexyl or cyclopentyl;
R² and R⁴ are independently H or halo; and
R³ is unsubstituted phenyl or phenyl optionally substituted with one or more halo or hydroxy groups; and
wherein the compound decreases capacitative calcium entry by at least about 10% or more in cells.

36. The method of claim 27, 29, 30 or 35, wherein the duration of treatment of the compound is about one day to twelve months.

37-46. (canceled)
